(19) United States
(12) Patent Application Publication
(10) Pub. No.: US 2025/0186520 A1
(43) Pub. Date: Jun. 12, 2025

(54) ONCOLYTIC VIRUS COMPOSITIONS AND METHODS FOR THE TREATMENT OF CANCER

(71) Applicant: City of Hope, Duarte, CA (US)

(72) Inventors: Jianhua Yu, Duarte, CA (US); Michael A. Caligiuri, Duarte, CA (US); Lei Tian, Duarte, CA (US)

(21) Appl. No.: 18/033,768

(22) PCT Filed: Oct. 26, 2021
(86) PCT No.: PCT/US21/56703

§ 371 Date: Apr. 25, 2023

Related U.S. Application Data
(60) Provisional application No. 63/105,841, filed on Oct. 26, 2020.

(51) Int. Cl. A61K 35/763; A61P 35/00; C07K 14/52; C07K 16/28; C12N 15/86

(52) U.S. Cl. CPC A61K 35/763; A61P 35/00; C07K 14/521; C07K 16/2863; C12N 15/86; C07K 2317/732; C07K 2319/30; C12N 2710/16632; C12N 2710/16643

(57) ABSTRACT

Provided herein, inter alia, are oncolytic viruses that express recombinant proteins having bispecific activity. The recombinant proteins include a first fusion protein comprising a first Fc domain covalently linked to a chemokine domain, and a second Fc fusion protein comprising a second Fc domain covalently linked to an anticancer therapeutic antibody domain. The recombinant proteins and oncolytic viruses provided herein are contemplated to be effective for the treatment of cancer, particularly by trafficking immune cells to the tumor microenvironment.

### CROSS-REFERENCE TO RELATED APPLICATIONS

This application claims priority to U.S. Provisional Application No. 63/105,841, filed Oct. 26, 2020, which is hereby incorporated by reference in its entirety and for all purposes.

### REFERENCE TO A SEQUENCE LISTING, A TABLE OR A COMPUTER PROGRAM LISTING APPENDIX SUBMITTED AS AN ASCII TEXT FILE

The Sequence Listing written in file 048440-787001WO_SequenceListing_ST25.TXT, created on Oct. 26, 2021, 24,576 bytes, machine format IBM-PC, MS Windows operating system, is hereby incorporated by reference.

### BACKGROUND

Glioblastoma (GBM) is the most aggressive and fatal primary brain tumor, with incidence increasing by 3% each year (1). GBM infiltrative growth and necrosis hamper the effectiveness of current therapies with surgery, chemotherapy, and radiotherapy. Lately, immunotherapy appears as a promising therapeutic approach.

“Hot” tumors, which are characterized by lymphocyte infiltration into the tumor microenvironment (TME), are increasingly recognized as better targets for immunotherapy than “cold” tumors, which contain few lymphocytes (2,3). Thus, there is considerable interest in novel approaches to make “cold” tumors “hot.” One area of focus is the chemokines, which attract cells of the innate and adaptive immune system to tumor sites. In GBM, intra-tumoral T lymphocyte number and function are reduced, and the number of monocytes and dendritic cells is also reduced, making them “cold” tumors (4-6).

GBM-related systemic immunosuppression has been well documented (2, 3). It has been reported that in GBM patients, T lymphocyte number and function are reduced, and the number of monocytes and dendritic cells are also down-regulated (4-6). Chemokines regulate immune cell trafficking in tumors and are implicated in tumor development, progression, and angiogenesis. C-C motif chemokine ligand 5 (CCL5) is an inflammatory chemokine that promotes chemotaxis on cells involved in the immune/inflammatory response. It has been reported that CCL5 is chemotactic for T cells, macrophages, dendritic cells, and natural killer (NK) cells through the expression of CCR1 and/or CCR5 (7-9). Furthermore, due to DNA methylation, tumor evolutionary pressure may lead to CCL5 expression silencing in many solid tumors (10). Thus, recovering or enhancing CCL5 expression in the tumor microenvironment (TME) might be a promising therapy on treating solid tumors. Difficulties with locoregional delivery into the TME and the short half-life of CCL5 have limited its usefulness as a cancer therapy.

Provided herein, inter alia, are solutions to these and other problems in the art.

### BRIEF SUMMARY OF THE INVENTION

In an aspect, is provided a recombinant protein including: a) a first Fc fusion protein including a first Fc domain covalently attached to a chemokine domain; and b) a second Fc fusion protein including a second Fc domain covalently attached to an anticancer therapeutic antibody domain, wherein the first Fc domain is capable of binding to the second Fc domain.

In an aspect, is provided a nucleic acid encoding a recombinant protein provided herein including embodiments thereof.

In an aspect, is provided an oncolytic virus including a nucleic acid encoding a recombinant protein including a) a first Fc fusion protein including a first Fc domain covalently attached to a chemokine domain and b) a second Fc fusion protein including a second Fc domain covalently attached to an anticancer therapeutic antibody domain, where the first Fc domain is capable of binding to the second Fc domain.

In an aspect is provided an oncolytic virus including an expression cassette encoding a recombinant protein including a) a first Fc fusion protein including a first Fc domain covalently attached to a chemokine domain and b) a second Fc fusion protein including a second Fc domain covalently attached to an anticancer therapeutic antibody domain, wherein the first Fc domain is capable of binding to the second Fc domain.

In an aspect, is provided a pharmaceutical compositions including an oncolytic virus provided herein including embodiments thereof, and a pharmaceutically acceptable carrier.

In another aspect is provided a pharmaceutical composition including a recombinant protein provided herein including embodiments thereof, and a pharmaceutically acceptable carrier.

In an aspect is provided a method for killing a cancer cell the method including contacting the cancer cell with an oncolytic virus provided herein including embodiments thereof.

In an aspect is provided a method for killing cancer cells including administering to a subject an effective amount of any of the oncolytic virus provided herein including embodiments thereof or the pharmaceutical composition provided herein inlucding embodiments thereof.

In an aspect is provided a method of treating or preventing cancer in a subject in need thereof, the method including administering to the subject an effective amount of the oncolytic virus provided herein including embodiments thereof or pharmaceutical composition provided herein including embodiments thereof.

In an aspect is provided a method of inducing an immune response in a subject, the method including administering to a subject the oncolytic virus provided herein including embodiments thereof or the pharmaceutical composition provided herein including embodiments thereof.

### BRIEF DESCRIPTION OF THE DRAWINGS

FIGS. 1A-1G present data showing the construction of Cmab-CCL5 and OV-Cmab-CCL5. (FIG. 1A) Schematic of the Cmab-h(m)CCL5 bispecific fusion protein. (FIG. 1B) Fluorescence intensity of anti-Fc-APC, which detects Cmab-hCCL5 bound to wild type (wt)EGFR U251T2 GBM cells and EGFRvIII U87ΔEGFR GBM cell lines or the EGFR-A2780 human ovarian cancer cell line, as measured by flow cytometry. Cmab-CCL5 and the IgG1 isotype control were purified from lentivirus-infected CHO cells. (FIG. 1C) Fluorescence intensity of anti-hCCL5-APC, which detects biologically active hCCL5. The biologically active CCL5 in this form was tested by a flow cytometry assay. U251T2 cells, U87 ΔEGFR, and A2780 cells were stained with different doses of Cmab-CCL5 or IgG1 isotype for 30 min and then washed twice and stained with APC-conjugated anti-human CCL5 for 20 min. The stained cells were assessed by flow cytometry. Experiments in FIG. 1B and FIG. 1C were repeated 3 times with similar results (n=3 technical replicates). (FIG. 1D) Genetic maps of the oncolytic viruses used in this study. Top: wild type HSV-1. Middle: control oHSV, OV-Q1, with deletion of two copies of γ34.5, dysfunction of ICP6, and insertion of the GFP gene. Bottom: OV-Cmab-CCL5 showing the insertion of the gene encoding the “knob” and “hole”. The “knob” and “hole” are linked by T2A sequences and are driven by the viral pIE4/5 promoter. (FIG. 1E) Immunoblotting performed with concentrated supernatants of engineered CHO cells and oHSV-infected U251T2 human GBM cells. Lentiviral-infected CHO cells (CHO-Cmab-CCL5) were used to produce Cmab-CCL5. Ten ml supernatants were collected from cultured CHO-EV (empty vector) and CHO-Cmab-CCL5 cells, as well as OV-Q1-infected and OV-Cmab-CCL5-infected U251T2 human GBM cells at 24 hours after infection. Total proteins in the supernatants were extracted and tested by immunoblotting with anti-human Fc or anti-human CCL5 antibody. (FIG. 1F) Fc yields of OV-Cmab-hCCL5- and OV-Q1-infected GBM cells were tested by ELISA. U251T2 human GBM cells were infected with OV-Cmab-CCL5- and OV-Q1 at a MOI of 2. The supernatants of the infected cells were collected at different time points for ELISA. (FIG. 1G) hCCL5 yields of OV-Cmab-hCCL5- and OV-Q1-infected GBM cells were tested by ELISA. U251T2 human GBM cells were infected with OV-Cmab-hCCL5- and OV-Q1 at a MOI of 2. The supernatants of the infected cells were collected at different time points for ELISA. Experiments in FIG. 1F and FIG. 1G were repeated 3 times with similar results (n=3 technical replicates).

FIGS. 2A-211 present exemplary data showing that Cmab-hCCL5 promoted human immune cells in vitro. (FIGS. 2A-2D) The migration of natural killer (NK) cells (FIG. 2A), macrophages (FIG. 2B), CD4+ T cells (FIG. 2C), and CD8+ T cells (FIG. 2D) induced by Cmab-hCCL5, as measured by a transwell assay. Recombinant hCCL5 (rhCCL5) was used as a positive control. (FIG. 2E-211) The migration of NK cells (FIG. 2E), macrophages (FIG. 2F), CD4+ T cells (FIG. 2G), and CD8+ T cells (FIG. 211) induced by the supernatant from OV-Cmab-hCCL5- or OV-Q1-infected U251T2 GBM cells, as measured by transwell assay. 200 ng rhCCL5 was used as the positive control. All experiments were repeated by at least 3 donors with similar results. Error bars indicate the standard deviations of triplicates, and statistical analyses were performed by Student's t test *P≤0.05; **P≤0.01; ***P≤0.001; ****P≤0.0001.

FIGS. 3A-3F present exemplary data showing that Cmab-hCCL5 enhanced NK cell mediated ADCC and activation against EGFR tumor cells. Cytotoxicity of human primary NK cells against Cmab-hCCL5-treated U251T2, OV-CAR8, GBM30, and Gli36ΔEGFR (FIG. 3A) Antibody-dependent cellular cytotoxicity (ADCC) of human primary NK cells against Cmab-hCCL5-treated U251T2, GBM30, and Gli36ΔEGFR cells at effector/target (E/T) ratios of 40:1, 20:1 and 10:1, as measured in a 51Cr release assay. For U251T2 GBM cells, control vs. Cmab-hCCL5 5 μg/ml ***P≤0.001; control vs. Cmab-hCCL5 10 μg/ml ***P<0.001. For GBM30 GBM cells, control vs. Cmab-hCCL5 5 μg/ml ***P<0.001; control vs. Cmab-hCCL5 10 μg/ml **P≤0.01. For Gli36ΔEGFR GBM cells, control vs. Cmab-hCCL5 5 μg/ml *P<0.05; control vs. Cmab-hCCL5 10 μg/ml *P<0.05. A linear mixed model was used to account for the underlying variance and covariance structure (n=3 donors). (FIG. 3B) ADCC of human primary NK cells against U251T2, GBM30, Gli36ΔEGFR GBM cells and A2780 ovarian tumor cells that were treated with conditioned media from OV-Q1- or OV-Cmab-hCCL5-infected U251T2 GBM cells, as measured by ⁵¹Cr release as noted above. For U251T2 cells, OV-Q1 vs. OV-Cmab-hCCL5 ***P≤0.001. For GBM30 GBM cells, OV-Q1 vs. OV-Cmab-hCCL5 ***P≤0.001. For Gli36ΔEGFR GBM cells, OV-Q1 vs. OV-Cmab-hCCL5 ***P≤0.001. For A2780 cells, no significant differences among uninfected, OV-Q1, and OV-Cmab-hCCL5. A linear mixed model was used to account for the underlying variance and covariance structure (n=3 donors). (FIG. 3C) CD69 expression, as measured by flow cytometry, on human primary NK cells co-cultured with GBM30 cells that were pre-treated with Cmab-hCCL5. (FIG. 3D) CD69 expression, as measured by flow cytometry, on human primary NK cells co-cultured with GBM30 cells that were pre-treated with conditioned media from OV-Q1- or OV-Cmab-hCCL5-infected U251T2 GBM cells. (FIG. 3E) Granzyme B expression, as measured by flow cytometry, on human primary NK cells co-cultured with GBM30 cells that were pre-treated with Cmab-hCCL5. (FIG. 3F) Granzyme B expression, as measured by flow cytometry, on human primary NK cells co-cultured with GBM30 cells that were pre-treated with conditioned media from OV-Q1- or OV-Cmab-hCCL5-infected cells. Error bars indicate the standard deviations of triplicates. Experiments in c, d, e, and f were representative of three independent experiments, and statistical analyses were performed by one-way ANOVA with P values corrected for multiple comparisons by Bonferroni method multiple comparisons test (n=3 donors). ***P≤0.001; ****P≤0.0001.

FIGS. 4A-4C present exemplary data showing that Cmab-hCCL5 enhanced human macrophage ADCP against GBM cells. Examination of the effect of Cmab-hCCL5 purified from CHO cells on phagocytosis of GBM cells by human primary macrophages. (FIG. 4A) Antibody-dependent cellular phagocytosis (ADCP) of human macrophages induced by Cmab-hCCL5 targeting of GBM30 cells. Pre-labeled GBM30 cells were labeled with CFSE and co-cultured with human macrophages at a ratio of 2:1 in the presence of vehicle control or Cmab-hCCL5 at the dose of 10 μg/ml. Percentage of human macrophages uptake of labeled tumor cells (CD45+ and CFSE+) was measured by flow cytometry assay to evaluate the amount of phagocytosis. (FIG. 4B) ADCP of human macrophages induced by conditioned media from OV-Cmab-hCCL5-infected human GBM cells induce phagocytosis GBM30 cells. Pre-labeled GBM30 cells were labeled with CFSE and co-cultured with human macrophages at a ratio of 2:1 in the presence of supernatants from OV-Q1-, or OV-Cmab-hCCL5-infected U251T2 human GBM cells that were collected at 24 hours after infection. Percentage of human macrophages uptake of labeled tumor cells (CD45+ and CFSE+) was measured by flow cytometry assay to evaluate the amount of phagocytosis. (FIG. 4C) Cytokine mRNA expression levels, as measured by real-time RT-PCR, from human macrophages co-cultured with GBM30 cells at a ratio of 1:1 with or without Cmab-hCCL5 for 6 hours. All experiments were repeated using at least 3 donors with similar results. Error bars indicate the standard deviations of triplicates, and statistical analyses were performed by one-way ANOVA with P values corrected for multiple comparisons by Bonferroni method multiple comparisons test. *P≤0.05; **P≤0.01.

FIGS. 5A-5E present exemplary data showing the effectiveness of OV-Cmab-hCCL5 to improve in vivo oncolytic virotherapy in a xenograft GBM on an animal model. (FIG. 5A) Experimental timeline for in vivo studies using a one-cycle treatment. An orthotopic model of human GBM was established by i.e. injection of 1×10⁵ GBM30-FFL human GBM cells into NSG mice. Four days after tumor implantation, the mice received 1×10⁵ PBMCs isolated from one donor by i.v. injection per mouse. Five days after tumor implantation, the mice received an i.e. injection with OV-Cmab-hCCL5 or OV-Q1 at the dose of 2×10⁵ PFU per mouse, or saline as a placebo control. One day after virus injection, 1×10⁵ activated T cells isolated from the same donor were i.v. injected per mouse. (FIG. 5B) Luciferase imaging of GBM30-FFL GBM mice with the indicated treatments on Day 15 and Day 21. (FIG. 5C) Survival of GBM30 tumor-bearing mice treated with OV-Q1, OV-Cmab-hCCL5, or vehicle control as indicated in FIG. 5A. P<0.01 for OV-Cmab-hCCL5 vs. OV-Q1. P<0.01 for OV-Cmab-hCCL5 vs. saline and P<0.01 for saline vs. OV-Q1. Survival was estimated by the Kaplan-Meier method and compared by log-rank test (n=7 animals). (FIG. 5D) Experimental timeline for in vivo studies using a two-cycle treatment. (FIG. 5E) Survival of GBM30 tumor-bearing mice treated with OV-Q1, OV-Cmab-hCCL5, or vehicle control, as indicated in d. P<0.01 for OV-Cmab-hCCL5 vs. OV-Q1. P<0.01 for OV-Cmab-hCCL5 vs. saline and P<0.01 for saline vs. OV-Q1. Survival was estimated by the Kaplan-Meier method and compared by log-rank test (n=6 animals).

FIGS. 6A-6G present exemplary data showing that Cmab-mCCL5 promotes murine immune cell migration and activation; OV-Cmab-mCCL5 prolongs survival of glioblastoma-bearing mice in an immunocompetent mouse model. (FIG. 6A) Fc yields of OV-Cmab-mCCL5- and OV-Q1-infected GBM cells were tested by ELISA assay. CT2A-hEGFR cells were infected with OV-Cmab-mCCL5- and OV-Q1 at a MOI of 2. The supernatants of the infected cells were collected at different time points for ELISA. (FIG. 6B) mCCL5 yields of OV-Cmab-hCCL5- and OV-Q1-infected GBM cells were tested by ELISA. CT2A-hEGFR cells were infected with OV-Cmab-mCCL5- and OV-Q1 at a MOI of 2. The supernatants of the infected cells were collected at different time points for ELISA. CT2A-hEGFR cells were infected with OV-Q1 or OV-Cmab-hCCL5 at a MOI of 2. The supernatant of virus-infected cells was collected at 48 hpi and put into the bottom chamber of the transwell. CD8+ T cells (10⁶ cells/chamber), NK cells (10⁶ cells/chamber), and macrophages (5*10⁵ cells/chamber) (FIG. 6C) The migration of murine NK cells, macrophages, CD4+ T cells, and CD8+ T cells induced by the supernatant of OV-Cmab-mCCL5- or OV-Q1-infected U251T2 GBM cells, as measured using a transwell assay. Recombinant mCCL5 (rmCCL5) was a positive control. Statistical analyses were performed by one-way ANOVA with P values corrected for multiple comparisons by Bonferroni method multiple comparisons test (n=3 mice). *P<0.05; ***P<0.001; ****P<0.0001. (FIG. 6D) Cytotoxicity of mouse primary NK cells against CT2A-hEGFR cells that were treated with conditioned media from uninfected, OV-Q1- or OV-Cmab-mCCL5-infected CT2A-hEGFR cells, as measured by ⁵¹Cr release. OV-Q1 vs. OV-Cmab-mCCL5 *P<0.05. A linear mixed model was used to account for the underlying variance and covariance structure (n=3 mice). (FIG. 6E) CD69 expression, as measured by flow cytometry, on mouse primary NK cells co-cultured with CT2A-hEGFR cells that were pre-treated with conditioned media from OV-Q1- or OV-Cmab-mCCL5-infected CT2A-hEGFR cells. (FIG. 6F) ADCP of mouse macrophages induced by conditioned media from OV-Cmab-mCCL5-infected cells targeting CT2A-hEGFR cells, as measured by flow cytometry. (FIG. 6G) Survival of CT2A-hEFGR tumor-bearing mice treated with OV-Q1, OV-Cmab-mCCL5, or vehicle control. P<0.01 for OV-Cmab-mCCL5 vs. OV-Q1. P<0.001 for OV-Cmab-mCCL5 vs. saline and P<0.001 for saline vs. OV-Q1. Survival was estimated by the Kaplan-Meier method and compared by log-rank test (n=7 or 8 animals). Error bars indicate the standard deviations of triplicates. Experiments in c and d were representative of three independent experiments. Statistical analyses were performed by one-way ANOVA with P values corrected for multiple comparisons by Bonferroni method multiple comparisons test (n=3 mice). ****P<0.0001.

FIGS. 7A-7M show characterization of OV-Cmab-mCCL5 and present exemplary data showing that OV-Cmab-mCCL5 induces infiltration of innate and adaptive murine immune cells into GBM TME that contribute to tumor eradication. (FIG. 7A) Cmab-mCCL5 binding affinity purified from CHO cells were tested by a flow cytometry assay. CT2A-hEGFR cells were stained with 1 ug Cmab-mCCL5 or IgG1 isotype for 30 min and then washed twice and stained with APC-conjugated anti-human Fc for 20 min. (FIG. 7B) The biologically active mCCL5 in this form was tested by a flow cytometry assay. CT2A-hEGFR cells, U87 ΔEGFR, and A2780 cells were stained with different doses of Cmab-mCCL5 or IgG1 isotype for 30 min and then washed twice and stained with PE-conjugated anti-mouse CCL5 for 20 min. The stained cells were assessed by flow cytometry. (FIG. 7C) Immunoblotting performed with concentrated supernatants of engineered CHO cells and oHSV-infected CT2A-hEGFR cells. Lentiviral-infected CHO cells (CHO-Cmab-mCCL5) were used to produce Cmab-mCCL5. Ten ml supernatants were collected from cultured CHO-EV (empty vector) and CHO-Cmab-mCCL5 cells, as well as OV-Q1-infected and OV-Cmab-mCCL5-infected CT2A-hEGFR cells at 24 hours after infection. Total proteins in the supernatants were extracted and tested by immunoblotting with anti-human Fc or anti-mouse CCL5 antibody. (FIGS. 7D-7G) The migration of murine NK cells (FIG. 7D), macrophages (FIG. 7E), CD4+ T cells (FIG. 7F), and CD8+ T cells (FIG. 7G) induced by Cmab-mCCL5, as measured using a Transwell assay. Recombinant mCCL5 (rmCCL5) was a positive control. Error bars indicate the standard deviations of triplicates. Experiments in a-c were representative of at least 3 times with similar results. Experiments in f were repeated with three independent experiments with similar results and statistical analyses were performed by Student's t test (n=3 mice). **P<0.01; ***P<0.001. (FIG. 711) The total number of NK cells, macrophages, and T cells measured by flow cytometry. (FIG. 7I) The total number of CD4⁺ T cells, CD8⁺ T cell, and the ratio of CD4⁺ T cells/CD8⁺ T cells measured by flow cytometry. (FIG. 7J) CD69 expression in NK cells isolated from mouse brains 3 days after virus injection. Representative flow cytometric plots of each group are shown on the left and summary data are on the right (n=4 animals). Error bars indicate the standard deviations of triplicates, and statistical analyses were performed by one-way ANOVA with P values corrected for multiple comparisons by Bonferroni method multiple comparisons test (n=4 or 5 mice). *P≤0.05; **P≤0.01; ***P≤0.001; ****P≤0.0001. (FIG. 7K) The representative MRI images of a bilateral CT2A-hEGFR immunocompetent GBM model treated with saline, OV-Q1, or OV-Cmab-mCCL5 treatment only on the right side. (FIG. 7L) mCCL5 yields from OV-Cmab-mCCL5-infected GBM cells, as tested by ELISA. (FIG. 7M) Cmab yields from OV-Cmab-mCCL5-infected GBM cells, as tested by ELISA.

FIGS. 8A-8E present exemplary data showing that Cmab-mCCL5 promoted innate and adaptive immunocyte activation in vitro. (FIG. 8A) Cytotoxicity of mouse primary NK cells against Cmab-mCCL5 pre-treated CT2A-hEGFR cells, as measured by ⁵¹Cr release. Control vs. Cmab-hCCL5 ***P<0.001. A linear mixed model was used to account for the underlying variance and covariance structure (n=3 mice). (FIG. 8B) Mouse primary NK cells were co-cultured with CT2A-hEGFR cells that were pre-treated with Cmab-mCCL5 at the dose of 5 μg/ml for 30 min at a ratio of 1:1 for 6 hours. CD69 expression on NK cells were measured by flow cytometry. (FIG. 8C) CD69 expression, as measured by flow cytometry, on mouse primary NK cells co-cultured with Cmab-mCCL5 pre-treated CT2A-hEGFR cells. (FIG. 8D) ADCP of mouse macrophages induced by Cmab-mCCL5 targeting of CT2A-hEGFR cells. Pre-labeled CT2A-hEGFR cells were co-cultured with human macrophages in the presence of vehicle control or Cmab-mCCL5. The percentage of mouse macrophages that phagocytosed labeled tumor cells was measured by flow cytometry. (FIG. 8E) Cytokine RNA expression levels, as measured by real-time RT-PCR, from mouse macrophages co-cultured with CT2A-hEGFR cells at a ratio of 1:1 with or without Cmab-mCCL5 for 6 hours. Error bars indicate the standard deviations of triplicates. Experiments in b and c were representative of three independent experiments with similar data. Experiments in b-d, statistical analyses were performed by one-way ANOVA with P values corrected for multiple comparisons by Bonferroni method multiple comparisons test (n=3 mice). **P<0.01; ***P<0.001; ****P<0.0001.

FIGS. 9A-9G present exemplary data showing that OV-Cmab-mCCL5 promoted innate and adaptive immunocyte infiltration in the TME and improved outcome in an immunocompetent GBM model. (FIG. 9A) CT2A-hEGFR cells were i.e injected into immunocompetent wild-type C57BL/6 mice to establish a GBM immunocompetent mouse model at Day 0. At Day 5, the mice received an i.e. injection with OV-Cmab-mCCL5 or OV-Q1 at the dose of 2×10⁵ PFU per mouse, or saline as a placebo control. Mice were euthanized at Day 7 or Day 9, respectively. (FIGS. 9B-9G) The total number of immune cells (FIG. 9B) and the percentage of NK cells (FIG. 9C), macrophages (FIG. 9D) among lymphocyte cells. Percentable of NK cells, macrophages and T cells and T cells among lymphocyte cells (FIG. 9E). Percentage and total number of macrophages (FIG. 9F), NK cells, and T cells (FIG. 9G). Flow cytometry methods were used to detect cells. For FIGS. 9B and 9E, error bars indicate the standard deviations, and statistical analyses were performed by one-way ANOVA with P values corrected for multiple comparisons by Bonferroni method multiple comparisons test (n=4 or 5 mice). *P<0.05; **P<0.01; ***P<0.001; ****P<0.0001. Experiments in FIGS. 9C and 9D were representative of one mouse result.

FIGS. 10A-10D present exemplary data showing the combination of OV-Cmab-CCL5 with Temozolomide TMZ) demonstrated a synergetic effect in vitro and in vivo. (FIG. 10A) Gli36ΔEGFR cells were treated with 1 μM TMZ overnight, and the pre-treated cells were ⁵¹Cr-labeled and used as target cells. These cells were then incubated with 5 ug/ml Cmab-hCCL5 and vehicle for 30 min. The effector NK cells and target cells were then co-cultured at the ratios of 40:1, 20:1, and 10:1 for 4 hours to assay for cytotoxicity. Cytotoxicity was then measured in a ⁵¹Cr release assay. A linear mixed model was used to account for the underlying variance and covariance structure (n=3 donors). (FIG. 10B) Gli36ΔEGFR cells were treated with 1 μM TMZ overnight, and the pre-treated cells were 51Cr-labeled and used as target cells. These cells were incubated and treated with conditional media from OV-Q1- or OV-Cmab-hCCL5-infected U251T2 GBM cells. The effector NK cells and target cells were then co-cultured at the ratios of 40:1, 20:1, and 10:1 for 4 hours to assay for cytotoxicity. Cytotoxicity was then measured in a ⁵¹Cr release assay. (FIG. 10C) Treatment plan used for study and (FIG. 10D) percent survival was assessed.

FIGS. 11A-11E present exemplary data showing the construction and characterization of OV-Cmab-CCL5-HIK (hole-T2A-knob; instead of knob-T2A-hole, as shown in FIG. 1D). (FIG. 11A) Cmab-CCL5 binding affinity of the supernatant extracted from virus infected tumor cells were tested by a flow cytometry assay. U251T2 cells treated with conditional media from OV-Q1- or OV-Cmab-mCCL5-infected U251T2 cells for 30 min stained with APC-conjugated anti-human Fc for 20 min. (FIG. 11B) The biologically active CCL5 in this form was tested by a flow cytometry assay. U251T2 cells treated with conditional media from OV-Q1- or OV-Cmab-mCCL5-infected U251T2 cells for 30 min stained with APC-conjugated anti-human CCL5 for 20 min. The stained cells were assessed by flow cytometry. (FIG. 11C) Schematic map of oncolytic viruses used in this study. Top: genetic map of wild type HSV-1. Second: genetic map of control oHSV, OV-Q1, with deletion of two copies of γ34.5, dysfunction of ICP6, and insertion of the GFP gene. Third: genetic map of OV-OV-Cmab-CCL5 showing the inserted coding gene of “hole” and “knob”. The “hole” and “knob” were linked by a T2A sequences and were driven by the viral pIE4/5 promoter. (FIG. 11D) Cmab-CCL5 binding affinity purified from CHO cells were tested by a flow cytometry assay. U251T2 cells were stained with different does of Cmab-CCL5 or Cmab-CCL5-HIK for 30 min and then washed twice and stained with APC-conjugated anti-human Fc for 20 min. (FIG. 11E) The biologically active CCL5 in this form was tested by a flow cytometry assay. U251T2 cells, U87 ΔEGFR, and A2780 cells were stained with different doses of Cmab-CCL5 or Cmab-CCL5-HIK for 30 min and then washed twice and stained with APC-conjugated anti-human CCL5 for 20 min. The stained cells were assessed by flow cytometry.

FIGS. 12A-B present the oncolytic effects of the constructs. (FIG. 12A) Cytotoxicity of human primary NK cells against Cmab-hCCL5-treated OV CAR8 ovarian tumor cells. (FIG. 12B) Cytotoxicity of human primary NK cells against OV CAR8 cells that were treated with conditional media from OV-Q1- or OV-Cmab-hCCL5-infected OV CAR8 cells.

FIGS. 13A-13E present exemplary data showing CCL5 concentration secreted by tumor cells and CCL5 receptor expression on NK cells, macrophages, and T cells. (FIG. 13A) CCL5 concentration secreted by human GBM cell lines (U251T2, Gli36ΔEGFR and GBM30), as measured by ELISA. (FIGS. 13B-13E) The CCR1 and CCR5 expression on human NK cells (FIG. 13B), macrophages (FIG. 13C), CD4+ T cells (FIG. 13D), and CD8+ T cells (FIG. 13E), as measured by flow cytometry. Experiments were repeated for 3 times with similar results. Experiments in FIGS. 13B-E were representative of at least 3 donors with similar results. Error bars indicate the standard deviations of triplicates.

FIGS. 14A-14E present exemplary data showing characterization of OV-hCCL5-Cmab. (FIG. 14A) The binding affinity of OV-Cmab-hCCL5 to U251T2 cells, as measured by flow cytometry using anti-Fc-APC antibody. (FIG. 14B) The biologically active hCCL5 from OV-Cmab-hCCL5, as measured by flow cytometry using anti-hCCL5-APC antibody. (FIG. 14C) The binding affinity of Cmab-hCCL5 or CCL5-Cmab—to U251T2, as measured by flow cytometry using anti-Fc-APC antibody. (FIG. 14D) The biologically active hCCL5 from Cmab-hCCL5 or hCCL5-Cmab, as measured by flow cytometry using anti-hCCL5-APC antibody. (FIG. 14E) The ability of OV-Q1 and OV-Cmab-hCCL5 to induce oncolysis of GBM cells, as measured by real-time cell analysis. All experiments were repeated at least 3 times with similar results of triplicates. Experiments in a-d were representative of three independent experiments with similar results.

FIG. 15 presents quantification of luciferase expression in the GBM30-FFL mouse model. The luciferase intensity of the experimental mice shown in the main FIG. 5b was measured 21 days post tumor implantation. Data are presented for individual mice (n=7). Error bars represent standard deviations of all individual mice. *P<0.05; **P<0.01.

FIG. 16 presents gating strategy of GBM immunocompetent model.

FIGS. 17A-17D present exemplary data showing OV-Cmab-mCCL5 improved oncolytic virotherapy depended on NK cells, macrophages and T cells. (FIGS. 17A-17C) Survival of CT2A-hEGFR tumor-bearing C57BL/6J immunocompetent mice treated with vehicle control, OV-Q1, or OV-Cmab-mCCL5 after NK cell depletion (FIG. 17A), macrophage depletion (FIG. 17B), or T cell depletion (FIG. 17C). P<0.001 for the difference between OV-Cmab-mCCL5 with vs. without NK cell depletion and the difference of OV-Q1 with vs. without NK cell depletion (FIG. 17A). Survival was estimated by the Cox regression model (n=7 animals). P<0.001 for the difference between OV-Cmab-mCCL5 with vs. without macrophage depletion and the difference between OV-Q1 with vs. without macrophage depletion (b). Survival was estimated by the Cox regression model (n=7 animals). P<0.001 for the difference between OV-Cmab-mCCL5 with vs. without T cell depletion and the difference of OV-Q1 with vs. without T cell depletion (FIG. 17C). Survival was estimated by the Cox regression model (n=7 animals). (FIG. 17D) The scheme of a model in which OV-Cmab-CCL5 induces immune infiltration to destroy GBM tumors. Without OV treatment, the level of CCL5 is low in tumors and high in the other parts of the TME, which leads to tumor metastasis. However, high CCL5 expression from OV-Cmab-mCCL5-infected GBM cells results in gradient change of CCL5 with a high level in tumors while a low level in the other parts of TME, leading to immune cell migration towards tumors (FIG. 17D).

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the TUPAC-TUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., an Fc domain) in other proteins with different numbering systems. For example, by performing a simple sequence alignment with a protein (e.g., an Fc domain) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “EGFR protein” or “EGFR” as used herein includes any of the recombinant or naturally-occurring forms of epidermal growth factor receptor (EGFR) also known as ErbB-1 or HER1 in humans, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto.

The term “Her2 protein” or “Her2” as used herein includes any of the recombinant or naturally-occurring forms of Receptor tyrosine-protein kinase erbB-2, also known as CD340 (cluster of differentiation 340), proto-oncogene Neu, Erbb2 (rodent), or ERBB2 (human), or variants or homologs thereof that maintain Her2 activity (e.g. within at least 50%, 80%, 90%, 95%, 96%, 97%, 98%, 99% or 100% activity compared to Her2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Her2 protein. In embodiments, the Her2 protein is substantially identical to the protein identified by the UniProt reference number P04626 or a variant or homolog having substantial identity thereto.

The term “CD69” as referred to herein includes any of the recombinant or naturally-occurring forms of the Cluster of Differentiation 69 protein, or variants or homologs thereof that maintain CD69 activity (e.g. within at least 50%, 80%, 90%, 95%, 96%, 97%, 98%, 99% or 100% activity compared to CD69). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD69 protein. In embodiments, the CD69 protein is substantially identical to the protein identified by the UniProt reference number Q07108 or a variant or homolog having substantial identity thereto.

The term “CD20 protein” or “CD20” as used herein includes any of the recombinant or naturally-occurring forms of B-lymphocyte antigen CD20 or Cluster of Differentiation 20 (CD20), or variants or homologs thereof that maintain CD20 activity (e.g. within at least 50%, 80%, 90%, 95%, 96%, 97%, 98%, 99% or 100% activity compared to CD20). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD20 protein. In embodiments, the CD20 protein is substantially identical to the protein identified by the UniProt reference number P11836 or a variant or homolog having substantial identity thereto.

A “PD-1 protein” or “PD-1” as referred to herein includes any of the recombinant or naturally-occurring forms of the Programmed cell death protein 1 (PD-1) also known as cluster of differentiation 279 (CD 279) or variants or homologs thereof that maintain PD-1 protein activity (e.g. within at least 50%, 80%, 90%, 95%, 96%, 97%, 98%, 99% or 100% activity compared to PD-1 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-1 protein. In embodiments, the PD-1 protein is substantially identical to the protein identified by the UniProt reference number Q15116 or a variant or homolog having substantial identity thereto. In embodiments, the PD-1 protein is substantially identical to the protein identified by the UniProt reference number Q02242 or a variant or homolog having substantial identity thereto.

A “PD-L1” or “PD-L1 protein” as referred to herein includes any of the recombinant or naturally-occurring forms of programmed death ligand 1 (PD-L1) also known as cluster of differentiation 274 (CD 274) or variants or homologs thereof that maintain PD-L1 activity (e.g. within at least 50%, 80%, 90%, 95%, 96%, 97%, 98%, 99% or 100% activity compared to PD-L1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the UniProt reference number Q9NZQ7 or a variant or homolog having substantial identity thereto.

As used herein, the terms “VEGF protein” or “VEGF” or “vascular endothelial growth factor” are used in accordance with their plain ordinary meanings and refer to any of the recombinant or naturally-occurring forms of vascular endothelial growth factor (VEGF) or variants or homologs thereof that maintain VEGF activity (e.g. within at least 50%, 80%, 90%, 95%, 96%, 97%, 98%, 99% or 100% activity compared to VEGF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VEGF protein. In embodiments, the VEGF protein is substantially identical to the protein identified by the UniProt reference number Q9UNS8 or a variant or homolog having substantial identity thereto.

As used herein, the terms “VEGFR-1 protein” or “VEGFR1” or “vascular endothelial growth factor receptor 1” are used in accordance with their plain ordinary meanings and refer to any of the recombinant or naturally-occurring forms of vascular endothelial growth factor receptor 1 (VEGFR1) or variants or homologs thereof that maintain VEGFR1 activity (e.g. within at least 50%, 80%, 90%, 95%, 96%, 97%, 98%, 99% or 100% activity compared to VEGFR1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VEGFR1 protein. In embodiments, the VEGFR1 protein is substantially identical to the protein identified by the UniProt reference number P17948 or a variant or homolog having substantial identity thereto.

As used herein, the terms “VEGFR-2 protein” or “VEGFR2” or “vascular endothelial growth factor receptor 2” are used in accordance with their plain ordinary meanings and refer to any of the recombinant or naturally-occurring forms of vascular endothelial growth factor receptor 1 (VEGFR2) or variants or homologs thereof that maintain VEGFR2 activity (e.g. within at least 50%, 80%, 90%, 95%, 96%, 97%, 98%, 99% or 100% activity compared to VEGFR2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VEGFR2 protein. In embodiments, the VEGFR2 protein is substantially identical to the protein identified by the UniProt reference number P35968 or a variant or homolog having substantial identity thereto.

As used herein, the terms “PDGFR protein” or “PDGFR” are used in accordance with their plain ordinary meanings and refer to any of the recombinant or naturally-occurring forms of platelet-derived growth factor receptor (PDGFR) or variants or homologs thereof that maintain PDGFR activity (e.g. within at least 50%, 80%, 90%, 95%, 96%, 97%, 98%, 99% or 100% activity compared to PDGFR). In some aspects, the variants or homologs have at least 90%, 95%, 96%, 97%, 98%, 99% or 100% amino acid sequence identity across the whole sequence or a portion of the sequence (e.g.a 50, 100, 150 or 200 continuous amino acid portion) compared to a naturally occurring EGFR protein. In embodiments, the PDGFR protein is substantially identical to the protein PDGFR-alpha, identified by the UniProt reference number P16234 or a variant or homolog having substantial identity thereto. In embodiments, the PDGFR protein is substantially identical to the protein PDGFR-beta identified by the UniProt reference number P09619 or a variant or homolog having substantial identity thereto.

Nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Operably linked means that the nucleotide sequences being linked are typically contiguous. However, as enhancers generally function when separated from the promoter by several kilobases and intronic sequences may be of variable lengths, some polynucleotide elements may be operably linked but not directly flanked and may even function in trans from a different allele or chromosome. Linking may be accomplished by ligation at convenient sites. If such sites do not exist, the synthetic oligonucleotide adaptors or linkers are used in accordance with conventional practice.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

As used herein, the term “expression cassette” refers to a distinct component of vector DNA including a gene and regulatory sequence to be expressed by a transfected cell. In each successful transformation, the expression cassette directs the cell's machinery to make RNA and protein(s). Some expression cassettes are designed for modular cloning of protein-encoding sequences so that the same cassette can easily be altered to make different proteins. An expression cassette is composed of one or more genes and the sequences controlling their expression. An expression cassette comprises three components: a promoter sequence, an open reading frame, and a 3′ untranslated region that, in eukaryotes, usually contains a polyadenylation site. Different expression cassettes can be transfected into different organisms including bacteria, yeast, plants, and mammalian cells as long as the correct regulatory sequences are used.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods. In another example, a “recombinant virus” is a virus produced by recombining pieces of nucleic acid (e.g. DNA) using recombinant nucleic acid technology. Thus, a “recombinant oncolytic virus” is an oncolytic virus produced by recombining pieces of nucleic acid (e.g. DNA) from an oncolytic virus genome using recombinant nucleic acid technology.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the term “expression” is used in accordance with its plain ordinary meaning and refers to any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

Antibodies are large, complex molecules (molecular weight of ˜150,000 Da or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site (paratope), which docks onto the target antigen (epitope). The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially a Fab with part of the hinge region (see Fundamental Immunology (Paul ed., 3rd ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) or light chain variable region and variable heavy chain (VH) or heavy chain variable region refer to these light and heavy chain regions, respectively. The terms variable light chain (VL) and light chain variable region as referred to herein may be used interchangeably. The terms variable heavy chain (VH) and heavy chain variable region as referred to herein may be used interchangeably. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

The term “BiTe” or “bispecific antibody” as provided herein is used according to its conventional meaning well known in the art and refers to a bispecific recombinant protein capable to bind to two different antigens. For example, simultaneously. In contrast to traditional monoclonal antibodies, BiTE antibodies consist of two independently different antibody regions (e.g., two single-chain variable fragments (scFv)), each of which binds a different antigen. One antibody region may engage effector cells (e.g., T cells) by binding an effector cell-specific antigen (e.g., CD3 molecule) and the second antibody region may bind a target cell (e.g., cancer cell or autoimmune-reactive cell) through a cell surface antigen (e.g., EGFR) expressed by said target cell. Binding of the BiTE to the two antigens will link the effector cell (e.g., T cell) to the target cell (e.g., tumor cell) and activate the effector cell (e.g., T cell) via effector cell-specific antigen signaling (e.g., CD3 signaling). The activated effector cell (e.g., T cell) will then exert cytotoxic activity against the target cell (e.g., tumor cells).

The term “antigen” as provided herein refers to molecules (e.g. EGFR, HER-2, etc.) capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (CH, CL, VH, and VL, respectively). The paratope or antigen-binding site is formed on the N-terminus of the antigen binding domain. The two variable domains of an antigen binding domain typically bind the epitope on an antigen.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the antibody domains bind to a particular protein at least two times the background and more typically more than 10 to 100 times background. Specific binding to an antibody domain under such conditions requires an antibody domain that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

As used herein, the terms “immunoglobulin domain” or “Ig-domain” are used in accordance with their plain and ordinary meanings and refer to any of the recombinant or naturally-occurring forms of the Ig-domain or variants or homologs thereof that maintain the Ig-domain fold or three-dimensional structure. The Ig-domain belongs to a family of protein folds that consist of a 2-layer sandwich of 7-9 antiparallel beta-strands arranged in two beta-sheets with a Greek-key topology. The folding pattern typically consists of (N-terminal beta-hairpin-in sheet 1)-(beta-haripin-in sheet 2)-(beta-strand in sheet 1)-(C-terminal beta-hairpin in sheet 2) linkages. Immunoglobulin domains are the primary components of antibodies, and a large set of extracellular surface receptors, including receptor tyrosine kinases.

An example of an immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), V_{L} (light chain variable region), V_{H} (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001) As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries.

As used herein, the term “chimeric antibody” is used in accordance with its plain ordinary meaning and refers to an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

As used herein, the terms “Immunoglobulin G” or “IgG” are used in accordance with their plain and ordinary meanings and refer to any of the recombinant or naturally-occurring forms of the IgG antibody protein or variants or homologs thereof that maintain IgG activity (e.g. within at least 50%, 80%, 90%, 95%, 96%, 97%, 98%, 99% or 100% activity compared to IgG). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IgG polypeptide. IgG antibodies are composed of four separate chains (two identical light chains and two identical heavy chains) that form a homodimer (via inter-heavy chain disulfide) of heterodimers (one light chain and one heavy chain interchain disulfide) in a canonical Y-shaped quaternary structure. The light chain includes a variable immunoglobulin domain (V_{L}) and a constant immunoglobulin domain (C_{L}). The heavy chain includes one variable immunoglobulin domain (V_{H}) and three constant immunoglobulin domains (C_{H}1, C_{H}2, C_{H}3). The variable domains form the antigen-recognition surface of an IgG antibody. There are four subclasses of IgG, IgG1, IgG2, IgG3, and IgG4 that are different by function. IgG1 mediates thymus-related immune responses against polypeptide and protein antigens. IgG2 mediates immune responses toward polysaccharide or carbohydrate antigens. IgG3 mediates high-affinity responses against proteins and polypeptide antigens. IgG4 has a role in responses associated with food allergies, but its function is largely unknown.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the terms “natural killer cells” and “NK cells” are used in accordance with their plain ordinary meaning and refer to a type of cytotoxic lymphocyte involved in the innate immune system. The role NK cells play is typically analogous to that of cytotoxic T cells in the vertebrate adaptive immune response. NK cells may provide rapid responses to virus-infected cells, acting at around 3 days after infection, and respond to tumor formation. Typically, immune cells detect major histocompatibility complex (MHC) presented on infected cell surfaces, triggering cytokine release, causing lysis or apoptosis. NK cells typically have the ability to recognize stressed cells in the absence of antibodies and MHC, allowing for a much faster immune reaction.

As used herein, the term “macrophage” is used in accordance with its plain ordinary meaning and refers to a type of lymphocyte that engulfs and digests cellular debris, foreign substances, microbes, cancer cells, and anything else that does not have the type of proteins specific to healthy body cells on its surface in a process called phagocytosis. Beyond increasing inflammation and stimulating the immune system, macrophages also play an important anti-inflammatory role and can decrease immune reactions through the release of cytokines.

As used herein, the term “T cells” or “T lymphocytes” are used in accordance with their plain ordinary meaning and refer to a type of lymphocyte (a subtype of white blood cell) involved in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell, which results in the lysis of the host cell. A “replication-competent” virus as provided herein refers to a virus (herpes virus) that is capable of replicating in a cell (e.g., a cancer cell).

The terms “virus” or “virus particle” are used according to its plain ordinary meaning within virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “plaque forming units” is used according to its plain ordinary meaning in Virology and refers to the amount of plaques in a cell monolayer that can be formed per volume of viral particles. In some embodiments the units are based on the number of plaques that could form when infecting a monolayer of susceptible cells. For example, in embodiments 1,000 PFU/μl indicates that 1 μl of a solution including viral particles contains enough virus particles to produce 1000 infectious plaques in a cell monolayer. In embodiments, plaque forming units are abbreviated “PFU”.

The terms “multiplicity of infection” or “MOI” are used according to its plain ordinary meaning in Virology and refers to the ratio of infectious agent (e.g., poxvirus) to the target (e.g., cell) in a given area or volume. In embodiments, the area or volume is assumed to be homogenous.”

As used herein, the term “oncolytic virus” is a virus that preferentially infects and kills cancer cells. As the infected cancer cells are destroyed by oncolysis, they release new infectious virus particles or virions to help destroy the remaining tumor. In embodiments, oncolytic viruses are only to cause direct destruction of the tumor cells, but also stimulate host anti-tumor immune system responses. Examples of oncolytic viruses include adenovirus, herpes simplex virus, maraba virus, measles virus, Newcastle virus, picornavirus, reovirus, vaccinia virus, vesicular stomatitis virus, rubeloa virus, and myxoma virus. Thus in embodiments, the oncolytic virus is a adenovirus, reovirus, measles, herpes simplex, Newcastle disease virus, or vaccinia virus. In embodiments, the recombinant oncolytic virus is an adenovirus. In embodiments, the recombinant oncolytic virus is reovirus. In embodiments, the recombinant oncolytic virus is measles virus. In embodiments, the recombinant oncolytic virus is a Newcastle disease virus. In embodiments, the recombinant oncolytic virus is vaccinia virus. In embodiments, the recombinant oncolytic virus is an oncolytic herpes simplex virus (oHSV). In embodiments, the oHSV is a herpes simplex 1 virus. In embodiments, the oHSV is a herpes simplex 2 virus.

As used herein, the term “herpes simplex virus” or “HSV” refers to members of the Herpesviridae family. Herpes simplex virus 1 and 2 (HSV-1 and HSV-2), also known by their taxonomical names Human alphaherpesvirus 1 and Human alphaherpesvirus 2, are two members of the human Herpesviridae family, a set of viruses that produce viral infections in the majority of humans. Both HSV-1 (which produces most cold sores) and HSV-2 (which produces most genital herpes) are common and contagious.

As used herein, the term “promoter” refers to a sequence of DNA which proteins bind to initiate gene expression. For example, transcription factors may bind a promoter region of a gene to transcribe RNA from DNA.

As used herein, the term “γ34.5 gene” or “gamma-34.5 gene” refers to a herpes simplex virus gene whose function blocks the host cellular stress response to infection.

As used herein, the term “ICP6” or “ICP6 gene” refer to a gene that encodes a viral ribonucleotide reductase (vRR) function that allows replication of wild-type herpes simplex virus (HSV) to occur even in quiescent cells, such as the neurons. Without this function, HSV replication may be severely curtailed in quiescent cells. However, cycling cells upregulate expression of S-phase specific genes such as mRR for their own nucleic acid metabolism, which complements the defective viral ICP6 function, thus allowing these mutant HSVs to replicate. This complementation provides a biologic rationale for employment of ICP6-defective HSV in oncolytic therapy, rationale based on their preferential replication in cycling versus quiescent cells.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

As defined herein, the terms “activation”, “activate”, “activating”, “activator” and the like are used in accordance with its plain ordinary meaning and refer to an interaction that positively affects (e.g. increasing) the activity or function of a protein or cell relative to the activity or function of the protein or cell in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein that is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

As used herein, the terms “agonist,” “activator,” “upregulator,” etc. are used in accordance with its plain ordinary meaning and refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As used herein, the terms “inhibition”, “inhibit”, “inhibiting” and the like are used in accordance with its plain ordinary meaning and refer to an interaction that negatively affecting (e.g. decreasing) the activity or function of the protein or cell relative to the activity or function of the protein or cell in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein or cell from an indirect interaction (e.g. an inhibitor binds to a protein that activates the target protein, thereby preventing target protein activation or cell activations).

As used herein, the terms “inhibitor,” “repressor” or “antagonist” or “downregulator” are used in accordance with its plain ordinary meaning and refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “contacting” may include allowing two species to react, interact, or physically touch, wherein the two species may be, for example, an virus as described herein and a cancer cell. In embodiments contacting includes, for example, allowing a virus as described herein to physically touch a cancer cell. In embodiments, contacting refers to two species being in close enough proximity wherein one of the species exerts a desired effect. For example, contacting may refer to a recombinant protein provided herein being in close enough proximity to an immune cell wherein the chemokine domain has a chemotaxis effect (e.g. directional movement) on the immune cell. Thus, in embodiments, “contacting” does not refer to two species physically touching.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The term “patient” or “subject in need thereof” is used in accordance with its plain ordinary meaning and refers to a living organism suffering from or prone to a disease (e.g. cancer) or condition that can be treated by administration of a composition, compound, or method as provided herein. Non-limiting examples include humans, other mammals, bovines, rats, mice, dogs, monkeys, goat, sheep, cows, deer, and other non-mammalian animals. In some embodiments, a patient is human. In embodiments, the subject has, had, or is suspected of having cancer.

The terms “disease” or “condition” refer to a state of being or health status of a patient or subject capable of being treated with the compounds or methods provided herein. The disease may be a cancer. In some further instances, “cancer” refers to human cancers, including glioblastoma.

The term “associated” or “associated with” in the context of a substance or substance activity or function associated with a disease (e.g. glioblastoma) is caused by (in whole or in part), or a symptom of the disease is caused by (in whole or in part) the substance or substance activity or function. When the term is used in the context of a symptom, e.g. a symptom being associated with a disease or condition, it means that a symptom can be indicative of the disease or condition present in the subject who shows the symptom.

As used herein, the term “cancer” is used in accordance with its plain ordinary meaning and refers to all types of cancer, neoplasm or malignant tumors found in mammals (e.g. humans), including leukemias, lymphomas, carcinomas and sarcomas. Examples of cancers that may be treated with a compound, composition, or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is glioblastoma, ovarian cancer, pancreatic cancer, leukemia, lymphoma, lung cancer, breast cancer, or brain metastatic tumor of a primary tumor.

The term “leukemia” is used in accordance with its plain ordinary meaning and refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Examples of leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

As used herein, the term “prevent” is used in accordance with its plain ordinary meaning and refers to a decrease in the occurrence of disease symptoms in a patient. The prevention may be complete (no detectable symptoms) or partial, such that fewer symptoms are observed than would likely occur absent treatment.

As used herein, a “symptom” of a disease includes any clinical or laboratory manifestation associated with the disease (e.g. cancer), and is not limited to what a subject can feel or observe.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “immune response” is used in accordance with its plain ordinary meaning and refers to a response by an organism that protects against disease. An immune response may prevent progression (e.g. tumor growth) of a disease. The response can be mounted by the innate immune system or by the adaptive immune system. Thus, in embodiments, an“immune response” may be an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination. In embodiments, the immune response is an “innate immune response”, in which proteins and cells may recognize conserved features of antigens/pathogens/pathogenic cells to prevent disease.

As used herein, the term “cytotoxicity” is used in accordance with its plain ordinary meaning and refers to quality of being toxic to cells. As used herein, the terms “antibody-dependent cellular cytotoxicity”. “ADCC”, and “antibody-dependent cell-mediated cytotoxicity” are used in accordance with their plain ordinary meanings and refer to an immune mechanism through which Fc receptor-bearing effector cells can recognize and kill antibody-coated target cells expressing tumor- or pathogen-derived antigens on their surface.

As used herein, the terms “antibody-dependent cellular phagocytosis” and “ADCP” are used in accordance with their plain ordinary meanings and refer to the mechanism by which antibody-opsonized target cells activate the Fc receptors on the surface of macrophages to induce phagocytosis, resulting the ingestion and degradation of the target cell. The macrophage Fc receptors refer to all classes of Fcγ receptors.

As used herein, the terms “adaptive immune response”, “acquired immune system”, and “specific immune system” are used in accordance with their plain ordinary meanings and refer to a subsystem of the overall immune system that is composed of specialized, systemic cells and processes that eliminate designated targets. The targets are designated by identification via immunological memory. Immunological memory is created when the immune system had previously encountered the immune assault, and retained a record of it.

As used herein, the terms “tumor microenvironment”, “TME”, and “cancer microenvironment” are used in accordance with its plain ordinary meaning and refer to the non-neoplastic cellular environment of a tumor, including blood vessels, immune cells, fibroblasts, cytokines, chemokines, non-cancerous cells present in the tumor, and proteins produced.

As used herein, the term “cytokine” is used in accordance with its plain ordinary meaning and refers to a broad category of small proteins (˜5-20 kDa) that are involved in cell signaling. Cytokines are peptides that typically cannot cross the lipid bilayer of cells to enter the cytoplasm. In embodiments, cytokines are involved in autocrine signaling, paracrine signaling and endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell.

As used herein, the term “immunotherapy” and “immunotherapeutic agent” are used in accordance with their plain ordinary meaning and refer to the treatment of disease by activating or suppressing the immune system. Immunotherapies designed to elicit or amplify an immune response are classified as activation immunotherapies, while immunotherapies that reduce or suppress are classified as suppression immunotherapies. Such immunotherapeutic agents include antibodies and cell therapy.

As used herein, the term “anticancer agent” and “anticancer therapy” are used in accordance with their plain ordinary meaning and refer to a molecule or composition (e.g. compound, peptide, protein, nucleic acid, drug, antagonist, inhibitor, modulator) or regimen used to treat cancer through destruction or inhibition of cancer cells or tissues. Anticancer therapy includes chemotherapy, radiation therapy, surgery, targeted therapy, immunotherapy, and cell therapy Anticancer agents and/or anticancer therapy may be selective for certain cancers or certain tissues. In some embodiments, an anti-cancer therapy is an immunotherapy. In embodiments, anticancer agent or therapy may include a checkpoint inhibitor. In embodiments, the anti-cancer agent or therapy is a cell therapy.

In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DAIE), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pembrolizumab nivolumab, atezolizumab, avelumab, durvalumab or the like.

As used herein, the term “administering” means oral administration, administration as a suppository, topical contact, intravenous, systemic, intracavitary, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent. As used herein, “systemic administration” refers to a route of administration into the circulatory system so that the entire body of a subject is affected. Systemic administration incldes enteral and parenteral administrion. As used herein, the term “intracavitary administration” refers to a route of administration within any natural, non-pathologic cavity.

As used herein, the term “co-administer” refers to a composition described herein that is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation).

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

As used herein, the terms “pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Recombinant Protein Compositions

Compositions provided herein include recombinant proteins including a chemokine domain and an anticancer therapeutic antibody domain. The recombinant proteins have a bispecific functionality, including chemokine activity (e.g. inducing chemotaxis (e.g. inducing directional movement)) and binding of target cells (e.g. cancer cells). In embodiments, the anticancer therapeutic antibody domain binds an antigen (e.g. EGFR, Her-2) expressed on a target cell. In embodiments, the chemokine domain increases migration of responsive cells (e.g. immune cells) to the target cell. Thus, in an aspect is provided a recombinant protein including: (a) a first Fc fusion protein including a first Fc domain covalently attached to a chemokine domain; and (b) a second Fc fusion protein including a second Fc domain covalently attached to an anticancer therapeutic antibody domain, wherein the first Fc domain is capable of binding to the second Fc domain.

The term “recombinant protein” refers to a protein that is expressed from a nucleic acid that is heterologous to the cell in which the protein is expressed. For example, a nucleic acid encoding the recombinant protein may be introduced into a cell by transfection or transduction methods, thereby allowing the cell to express the protein. In embodiments, the nucleic acid encoding the recombinant protein is generated by recombining pieces of nucleic acid (e.g. DNA). The nucleic acid encoding the protein may be introduced into the cell in a vector or a plasmid. In embodiments, the recombinant protein includes a complex including two or more recombinant proteins (e.g. a first Fc fusion protein and a second Fc fusion protein). In embodiments, the recombinant protein includes a complex including two or more recombinant proteins (e.g. a first Fc fusion protein and a second Fc fusion protein), wherein the two or more recombinant proteins are attached by non-covalent bonds (e.g. hydrogen bonds). In embodiments, the recombinant protein includes a complex including two or more recombinant proteins (e.g. a first Fc fusion protein and a second Fc fusion protein), wherein the two or more recombinant proteins are attached by covalent bonds (e.g. disulfide bonds). Thus, in embodiments, the recombinant protein includes a first Fc fusion protein and a second Fc fusion protein, wherein the first Fc domain is bound to the second Fc domain by covalent bonds (e.g. disulfide bonds). In embodiments, the recombinant protein includes a first Fc fusion protein and a second Fc fusion protein, wherein the first Fc domain is bound to the second Fc domain by covalent bonds (e.g. disulfide bonds) and non-covalent bonds (e.g. hydrogen bonds).

A “fusion protein” refers to a protein encoding two or more separate protein sequences. In embodiments, the two or more separate protein sequences are recombinantly expressed as a single moiety. In embodiments, the two or more separate protein sequences are recombinantly expressed as separate moieties and are subsequently attached to generate a single moiety.

In embodiments, where the first Fc domain is covalently attached to a chemokine domain, the covalent attachment is through a peptide linker (e.g. an amino acid sequence that links that first Fc doming to the chemokine domain). In embodiments, where the second Fc domain is covalently attached to an anticancer therapeutic antibody domain, the covalent attachment is through a peptide linker (e.g. an amino acid sequence that links that first Fc doming to the chemokine domain). Peptide linkers may independently be from 2 amino acids to 500 amino acids in length. In embodiments, the peptide linkers are independently from 5 to 100 amino acids in length. In embodiments, the peptide linkers are independently from 5 to 50 amino acids in length. In embodiments, the peptide linkers are independently less than 500 amino acids in length. In embodiments, the peptide linkers are independently less than 250 amino acids in length. In embodiments, the peptide linkers are independently less than 100 amino acids in length. In embodiments, the peptide linkers are independently less than 90 amino acids in length. In embodiments, the peptide linkers are independently less than 80 amino acids in length. In embodiments, the peptide linkers are independently less than 70 amino acids in length. In embodiments, the peptide linkers are independently less than 60 amino acids in length. In embodiments, the peptide linkers are independently less than 50 amino acids in length. In embodiments, the peptide linkers are independently less than 40 amino acids in length. In embodiments, the peptide linkers are independently less than 30 amino acids in length. In embodiments, the peptide linkers are independently less than 25 amino acids in length. In embodiments, the peptide linkers are independently less than 20 amino acids in length. In embodiments, the peptide linkers are independently less than 15 amino acids in length. In embodiments, the peptide linkers are independently less than 10 amino acids in length.

In embodiments, the chemokine domain is attached to the N-terminus of the first Fc domain. In embodiments, the chemokine domain is attached to the N-terminus of the first Fc domain by a peptide linker. In embodiments, the peptide linker includes the sequence of SEQ ID NO:39. In embodiments, the peptide linker is the sequence of SEQ ID NO:39.

In embodiments, the anticancer therapeutic domain is attached to the N-terminus of the second Fc domain. In embodiments, the anticancer therapeutic domain is attached to the N-terminus of the second Fc domain by a peptide linker. In embodiments, the peptide linker includes the sequence of SEQ ID NO:39. In embodiments, the peptide linker is the sequence of SEQ ID NO:39.

The term “antibody domain” as provided herein refers to a protein moiety that forms part of the recombinant protein provided herein including embodiments thereof that is capable of binding an antigen (epitope). In embodiments, the antibody domain is an antibody or functional fragment thereof, that is attached to the remainder of the recombinant protein provided herein. The antibody domain provided herein may include a domain of an antibody or fragment (e.g., Fab, scFv) thereof. Thus, the antibody region may include a light chain variable domain (VL) and/or a heavy chain variable domain (VH). In embodiments, the antibody region provided herein includes a light chain variable (VL) domain. In embodiments, the antibody region includes a heavy chain variable (VH) domain. In embodiments, the antibody domain is a Fab domain. In embodiments, the antibody domain is an scFv. Thus, the term “anticancer therapeutic antibody domain” refers to an antibody domain that specifically binds to an antigen expressed on a cancer cell. In embodiments, the anticancer therapeutic antibody domain is a Fab domain. In embodiments, the anticancer therapeutic antibody domain is an scFv.

The term “Fc domain” as provided herein includes at least one heavy chain constant domain (e.g. CH2, CH3, CH4) of an antibody, and does not include any antibody variable domains (e.g. VH). For example, an IgG Fc domain, an IgA Fc domain, or an IgD Fc domain may include the CH2 and CH3 of an IgG, an IgA, or IgD antibody, respectively. For example, an IgM Fc domain may include the CH2, CH3 and CH4 of an IgM antibody. In embodiments, a first Fc domain is bound to a second Fc domain. A first Fc domain may be bound to a second Fc domain by covalent attachment. In embodiments, a first cysteine residue in a first Fc domain may form a disulfide bond with a second cysteine residue in the second Fc domain. In embodiments, a first Fc domain is bound to a second Fc domain by a plurality of disulfide bonds. A first Fc domain may bind to a second Fc domain through non-covalent interactions. In embodiments, a first residue in the first Fc domain may form hydrogen bonds with a second residue in the second Fc domain. In embodiments, a first residue in the first Fc domain may form a hydrophobic interaction (e.g. van der Waals interaction) with a second residue of the second Fc domain.

In embodiments, the first Fc domain is different from the second Fc domain. In embodiments, the first Fc domain and the second Fc domain are independently a first IgG Fc domain and a second IgG Fc domain. In embodiments, the first Fc domain or the second Fc domain is an IgG Fc. In embodiments, the first Fc domain is a first IgG Fc domain. In embodiments, the second Fc domain is a second IgG Fc domain.

In embodiments, the first IgG Fc domain is a first IgG1 Fc domain and the second IgG Fc domain is a second IgG1 Fc domain.

In embodiments, the first IgG Fc domain is a first IgG1 Fc domain, a first IgG2 Fc domain, a first IgG3 Fc domain, or a first IgG4 Fc domain. In embodiments, the first IgG Fc domain is a first IgG1 Fc domain. In embodiments, the first IgG Fc domain is a first IgG2 Fc domain. In embodiments, the first IgG Fc domain is a first IgG3 Fc domain. In embodiments, the first IgG Fc domain is a first IgG4 Fc domain.

In embodiments, the second IgG Fc domain is a second IgG1 Fc domain, a second IgG2 Fc domain, a second IgG3 Fc domain, or a second IgG4 Fc domain. In embodiments, the second IgG Fc domain is a second IgG1 Fc domain. In embodiments, the second IgG Fc domain is a second IgG2 Fc domain. In embodiments, the second IgG Fc domain is a second IgG3 Fc domain. In embodiments, the second IgG Fc domain is a second IgG4 Fc domain.

In embodiments, the first Fc domain and the second Fc domain include residues that inhibit homodimerization. In embodiments, a “knob-into-hole” strategy may be adopted to increase heterodimerization of a first Fc domain and a second Fc domain. For example, a first Fc domain knob may be generated by substituting amino acid residues with small side chains with residues with large side chains (e.g. tyrosine, tryptophan), and a second Fc domain hole may be generated by substituting residues with large side chains with residues with small side chains (e.g. alanine, threonine). Thus, a “knob” on a first Fc domain may be inserted into a “hole” in a second Fc domain. In embodiments, a residue in a first Fc domain may be substituted with a first cysteine and a residue in a second Fc domain may be substituted with a second cysteine residue to generate disulfide bonds between a first Fc domain and a second Fc domain. Thus, in embodiments, a first Fc domain is attached to a second Fc domain by a disulfide bond.

In embodiments, the first Fc domain includes a cysteine at a position corresponding to position 349 of SEQ ID NO:7, a serine at a position corresponding to position 366 of SEQ ID NO:7, an alanine at a position corresponding to position 368 of SEQ ID NO:7, a valine at a position corresponding to position 407 of SEQ ID NO:7, and/or a lysine at a position corresponding to position 405 of SEQ ID NO:7. In embodiments, the first Fc domain includes a cysteine at a position corresponding to position 349 of SEQ ID NO:7, a serine at a position corresponding to position 366 of SEQ ID NO:7, an alanine at a position corresponding to position 368 of SEQ ID NO:7, a valine at a position corresponding to position 407 of SEQ ID NO:7, and a lysine at a position corresponding to position 405 of SEQ ID NO:7. In embodiments, the first Fc domain includes a cysteine at a position corresponding to position 349 of SEQ ID NO:7. In embodiments, the first Fc domain includes a serine at a position corresponding to position 366 of SEQ ID NO:7. In embodiments, the first Fc domain includes an alanine at a position corresponding to position 368 of SEQ ID NO:7. In embodiments, the first Fc domain includes a valine at a position corresponding to position 407 of SEQ ID NO:7. In embodiments, the first Fc domain includes a lysine at a position corresponding to position 405 of SEQ ID NO:7

In embodiments, the second Fc domain includes a cysteine at a position corresponding to position 354 of SEQ ID NO:7, a tryptophan at a position corresponding to position 366 of SEQ ID NO:7, and/or an alanine at a position corresponding to position 409 of SEQ ID NO:7. In embodiments, the second Fc domain includes a cysteine at a position corresponding to position 354 of SEQ ID NO:7, a tryptophan at a position corresponding to position 366 of SEQ ID NO:7, and an alanine at a position corresponding to position 409 of SEQ ID NO:7. In embodiments, the second Fc domain includes a cysteine at a position corresponding to position 354 of SEQ ID NO:7. In embodiments, the second Fc domain includes a tryptophan at a position corresponding to position 366 of SEQ ID NO:7. In embodiments, the second Fc domain includes an alanine at a position corresponding to position 409 of SEQ ID NO:7.

In embodiments, the first Fc domain includes a cysteine at a position corresponding to position 354 of SEQ ID NO:7, a tryptophan at a position corresponding to position 366 of SEQ ID NO:7, and/or an alanine at a position corresponding to position 409 of SEQ ID NO:7. In embodiments, the first Fc domain includes a cysteine at a position corresponding to position 354 of SEQ ID NO:7, a tryptophan at a position corresponding to position 366 of SEQ ID NO:7, and an alanine at a position corresponding to position 409 of SEQ ID NO:7. In embodiments, the first Fc domain includes a cysteine at a position corresponding to position 354 of SEQ ID NO:7. In embodiments, the first Fc domain includes a tryptophan at a position corresponding to position 366 of SEQ ID NO:7. In embodiments, the first Fc domain includes an alanine at a position corresponding to position 409 of SEQ ID NO:7.

In embodiments, the second Fc domain includes a cysteine at a position corresponding to position 349 of SEQ ID NO:7, a serine at a position corresponding to position 366 of SEQ ID NO:7, an alanine at a position corresponding to position 368 of SEQ ID NO:7, a valine at a position corresponding to position 407 of SEQ ID NO:7, and/or a lysine at a position corresponding to position 405 of SEQ ID NO:7. In embodiments, the second Fc domain includes a cysteine at a position corresponding to position 349 of SEQ ID NO:7, a serine at a position corresponding to position 366 of SEQ ID NO:7, an alanine at a position corresponding to position 368 of SEQ ID NO:7, a valine at a position corresponding to position 407 of SEQ ID NO:7, and a lysine at a position corresponding to position 405 of SEQ ID NO:7. In embodiments, the second Fc domain includes a cysteine at a position corresponding to position 349 of SEQ ID NO:7. In embodiments, the second Fc domain includes a serine at a position corresponding to position 366 of SEQ ID NO:7. In embodiments, the second Fc domain includes an alanine at a position corresponding to position 368 of SEQ ID NO:7. In embodiments, the second Fc domain includes a valine at a position corresponding to position 407 of SEQ ID NO:7. In embodiments, the second Fc domain includes a lysine at a position corresponding to position 405 of SEQ ID NO:7.

In embodiments, the first Fc domain includes the sequence of SEQ ID NO:38. In embodiments, the first Fc domain has at least 90% sequence identity to the sequence of SEQ ID NO:38. In embodiments, the first Fc domain has at least 95% sequence identity to the sequence of SEQ ID NO:38. In embodiments, the first Fc domain has at least 98% sequence identity to the sequence of SEQ ID NO:38. In embodiments, the first Fc domain is the sequence of SEQ ID NO:38. In embodiments, the second Fc domain includes the sequence of SEQ ID NO:40. In embodiments, the second Fc domain has at least 90% sequence identity to the sequence of SEQ ID NO:40. In embodiments, the second Fc domain has at least 95% sequence identity to the sequence of SEQ ID NO:40. In embodiments, the second Fc domain has at least 98% sequence identity to the sequence of SEQ ID NO:40. In embodiments, the second Fc domain is the sequence of SEQ ID NO:40.

In embodiments, the first Fc domain includes the sequence of SEQ ID NO:38 and the second Fc domain includes the sequence of SEQ ID NO:40. In embodiments, the first Fc domain is the sequence of SEQ ID NO:38 and the second Fc domain is the sequence of SEQ ID NO:40.

In embodiments, the first Fc domain includes the sequence of SEQ ID NO:40. In embodiments, the first Fc domain has at least 90% sequence identity to the sequence of SEQ ID NO:40. In embodiments, the first Fc domain has at least 95% sequence identity to the sequence of SEQ ID NO:40. In embodiments, the first Fc domain has at least 98% sequence identity to the sequence of SEQ ID NO:40. In embodiments, the first Fc domain is the sequence of SEQ ID NO:40. In embodiments, the second Fc domain includes the sequence of SEQ ID NO:38. In embodiments, the second Fc domain has at least 90% sequence identity to the sequence of SEQ ID NO:38. In embodiments, the second Fc domain has at least 95% sequence identity to the sequence of SEQ ID NO:38. In embodiments, the second Fc domain has at least 98% sequence identity to the sequence of SEQ ID NO:38. In embodiments, the second Fc domain is the sequence of SEQ ID NO:38.

In embodiments, the first Fc domain includes the sequence of SEQ ID NO:40 and the second Fc domain includes the sequence of SEQ ID NO:38. In embodiments, the first Fc domain is the sequence of SEQ ID NO:40 and the second Fc domain is the sequence of SEQ ID NO:38.

The term “chemokine” is used in accordance with its plain ordinary meaning, and refers to a family of signaling proteins that are capable of inducing chemotaxis (e.g. directional movement) in responsive cells. Cytokines may be classified as chemokines according to activity, for example their ability to mediate chemotaxis. Typically, chemokines are about 8-10 kilodaltons in mass and may have four cysteine residues in conserved regions. Chemokines may interact with G protein-linked transmembrane receptors called chemokine receptors; these receptors are typically found on the surfaces of target cells. Chemokines have been classified into four main subfamilies: CXC, CC, CX3C and XC. In embodiments, the recombinant proteins described herein include one or more of a CC type chemokine domain, a CXC type chemokine domain, a C type chemokine domain, and a CX3C type chemokine. Thus, a “chemokine domain” as used herein refers to a chemokine or a functional fragment thereof (e.g. attached to the remainder of the fusion protein as disclosed herein). In embodiments, a chemokine domain maintains at least 20%, 30%, 40%, 50%, 60%, 70%, 80%, 90%, 95%, 96%, 97%, 98%, 99% or 100% activity compared to a full-length chemokine.

For the recombinant protein provided herein, in embodiments, the chemokine domain is a CC type chemokine domain, a CXC type chemokine domain, a C type chemokine domain, or a CX3C type chemokine domain. In embodiments, the chemokine domain is a CC type chemokine domain. In embodiments, the chemokine domain is a CXC type chemokine domain. In embodiments, the chemokine domain is a C type chemokine domain. In embodiments, the chemokine domain is a CX3C type chemokine domain. In embodiments, the CC type chemokine domain is a CCL5 domain. In embodiments, the CCL5 domain has at least 80% sequence identity to the sequence of SEQ ID NO:1. In embodiments, the CCL5 domain has at least 85% sequence identity to the sequence of SEQ ID NO:1. In embodiments, the CCL5 domain has at least 90% sequence identity to the sequence of SEQ ID NO:1. In embodiments, the CCL5 domain has at least 95% sequence identity to the sequence of SEQ ID NO:1. In embodiments, the CCL5 domain has at least 96% sequence identity to the sequence of SEQ ID NO:1. In embodiments, the CCL5 domain has at least 97% sequence identity to the sequence of SEQ ID NO:1. In embodiments, the CCL5 domain has at least 98% sequence identity to the sequence of SEQ ID NO:1. In embodiments, the CCL5 domain has at least 99% sequence identity to the sequence of SEQ ID NO:1. In embodiments, the CCL5 domain includes the sequence of SEQ ID NO:1. In embodiments, the CCL5 domain is the sequence of SEQ ID NO:1.

In embodiments, the recombinant protein includes a CC type chemokine domain. In embodiments, the CC type chemokine domain is CCL2, CCL3, CCL4, CCL5, CCL8, CCL9, CCL10, CCL11, CCL13, CCL14, CCL18, CCL19, CCL20, CCL21, CCL25, or CCL27. In embodiments, the CC type chemokine domain is CCL2. In embodiments, the CC type chemokine domain is CCL3. In embodiments, the CC type chemokine domain is CCL4. In embodiments, the CC type chemokine domain is CCL5. In embodiments, the CC type chemokine domain is CCL8. In embodiments, the CC type chemokine domain is CCL9. In embodiments, the CC type chemokine domain is CCL10. In embodiments, the CC type chemokine domain is CCL11. In embodiments, the CC type chemokine domain is CCL14. In embodiments, the CC type chemokine domain is CCL19. In embodiments, the CC type chemokine domain is CCL20. In embodiments, the CC type chemokine domain is CCL21. In embodiments, the CC type chemokine domain is CCL25. In embodiments, the CC type chemokine domain is CCL27.

In embodiments, the CCL2 domain includes sequence of SEQ ID NO:2. In embodiments, the CCL2 domain has at least 80% sequence identity to the sequence of SEQ ID NO:2. In embodiments, the CCL2 domain has at least 85% sequence identity to the sequence of SEQ ID NO:2. In embodiments, the CCL2 domain has at least 90% sequence identity to the sequence of SEQ ID NO:2. In embodiments, the CCL2 domain has at least 95% sequence identity to the sequence of SEQ ID NO:2. In embodiments, the CCL2 domain has at least 98% sequence identity to the sequence of SEQ ID NO:2. In embodiments, the CCL2 domain is sequence of SEQ ID NO:2.

In embodiments, the CCL3 domain includes the sequence of SEQ ID NO:3. In embodiments, the CCL3 domain has at least 80% sequence identity to the sequence of SEQ ID NO:3. In embodiments, the CCL3 domain has at least 85% sequence identity to the sequence of SEQ ID NO:3. In embodiments, the CCL3 domain has at least 90% sequence identity to the sequence of SEQ ID NO:3. In embodiments, the CCL3 domain has at least 95% sequence identity to the sequence of SEQ ID NO:3. In embodiments, the CCL3 domain has at least 98% sequence identity to the sequence of SEQ ID NO:3. In embodiments, the CCL3 domain is the sequence of SEQ ID NO:3.

In embodiments, the CCL4 domain includes the sequence of SEQ ID NO:4. In embodiments, the CCL4 domain has at least 80% sequence identity to the sequence of SEQ ID NO:4. In embodiments, the CCL4 domain has at least 85% sequence identity to the sequence of SEQ ID NO:4. In embodiments, the CCL4 domain has at least 90% sequence identity to the sequence of SEQ ID NO:4. In embodiments, the CCL4 domain has at least 95% sequence identity to the sequence of SEQ ID NO:4. In embodiments, the CCL4 domain has at least 98% sequence identity to the sequence of SEQ ID NO:4. In embodiments, the CCL4 domain is the sequence of SEQ ID NO:4.

In embodiments, the recombinant proteins described herein include a CXC type chemokine domain. In embodiments, the CXC type chemokine domain is selected from CXCL8, CXCL10, CXCL12 and CXCL13. In embodiments, the CXC type chemokine domain is CXCL8. In embodiments, the CXC type chemokine domain is CXCL10. In embodiments, the CXC type chemokine domain is CXCL12. In embodiments, the CXC type chemokine domain is CXCL13.

In embodiments, the anticancer therapeutic antibody domain binds EGFR or Her-2. In embodiments, the anticancer therapeutic antibody domain binds EGFR. In embodiments, the anticancer therapeutic antibody domain binds Her-2. In embodiments, the anticancer therapeutic antibody domain binds PD-1, PDL1, VEGF, VEGFR2, PDGFRa, CD52, CD38, RANKL, GD2, SLAMF7, CCR4, CD20, or CD19. In embodiments, the anticancer therapeutic antibody domain binds PD-1. In embodiments, the anticancer therapeutic antibody domain binds PDL1. In embodiments, the anticancer therapeutic antibody domain binds VEGFR2. In embodiments, the anticancer therapeutic antibody domain binds PDGFRa. In embodiments, the anticancer therapeutic antibody domain binds CD52. In embodiments, the anticancer therapeutic antibody domain binds CD38. In embodiments, the anticancer therapeutic antibody domain binds RANKL. In embodiments, the anticancer therapeutic antibody domain binds GD2. In embodiments, the anticancer therapeutic antibody domain binds SLAMF7. In embodiments, the anticancer therapeutic antibody domain binds CCR4. In embodiments, the anticancer therapeutic antibody domain binds CD20. In embodiments, the anticancer therapeutic antibody domain binds CD19.

In embodiments, the anticancer therapeutic antibody domain is an anti-EGFR antibody domain or an anti-Her2 antibody domain. In embodiments, the anticancer therapeutic antibody domain is an anti-EGFR antibody domain. In embodiments, the anticancer therapeutic antibody domain is an anti-Her-2 antibody domain. In embodiments, the anti-EGFR antibody domain is a Fab domain or an scFv. In embodiments, the anti-EGFR antibody domain is a Fab domain. In embodiments, the anti-EGFR antibody domain is an scFv. In embodiments, the anti-EGFR antibody domain is a domain of cetuximab (Erbitux®). In embodiments, the anti-Her-2 antibody domain is a Fab domain or an scFv. In embodiments, the anti-Her-2 antibody domain is a Fab domain. In embodiments, the anti-Her-2 antibody domain is an scFv. In embodiments, the anti-Her-2 antibody domain is a domain of trastuzumab (Herceptin®).

In embodiments, the anticancer therapeutic antibody domain is a domain of alemtuzumab (Campath®). In embodiments, the anticancer therapeutic antibody domain is domain of bevacizumab (Avastin®). In embodiments, the anticancer therapeutic antibody domain is a domain of daratumumab (Darzalex®). In embodiments, the anticancer therapeutic antibody domain is a domain of denosumab (Xgeva®). In embodiments, the anticancer therapeutic antibody domain is a domain of dinutuximab (Unituxin®). In embodiments, the anticancer therapeutic antibody domain is a domain of elotuzumab (Empliciti®). In embodiments, the anticancer therapeutic antibody domain is a domain of isatuximab (Sarclisa®). In embodiments, the anticancer therapeutic antibody domain is a domain of mogamulizumab (Poteligeo®). In embodiments, the anticancer therapeutic antibody domain is a domain of necitumumab (Portrazza®). In embodiments, the anticancer therapeutic antibody domain is a domain of obinutuzumab (Gazyva®). In embodiments, the anticancer therapeutic antibody domain is a domain of ofatumumab (Arzerra®). In embodiments, the anticancer therapeutic antibody domain is a domain of olaratumumab (Lartruvo®) antibody. In embodiments, the anticancer therapeutic antibody domain is a domain of panitumumab (Vectibix®). In embodiments, the anticancer therapeutic antibody domain is domain of pertuzumab (Perjeta®). In embodiments, the anticancer therapeutic antibody domain is a domain of ramucirumab (Cyramza®). In embodiments, the anticancer therapeutic antibody domain is a domain of rituximab (Rituxan®). In embodiments, the anticancer therapeutic antibody domain is a domain of tafasitamab (Monjuvi®). In embodiments, the anticancer therapeutic antibody domain is a Fab domain. In embodiments, the anticancer therapeutic antibody domain is an scFv.

In embodiments, the anticancer therapeutic antibody domain includes the sequence of SEQ ID NO:37. In embodiments, the anticancer therapeutic antibody domain has at least 80% sequence identity to the sequence of SEQ ID NO:37. In embodiments, the anticancer therapeutic antibody domain has at least 85% sequence identity to the sequence of SEQ ID NO:37. In embodiments, the anticancer therapeutic antibody domain has at least 90% sequence identity to the sequence of SEQ ID NO:37. In embodiments, the anticancer therapeutic antibody domain has at least 95% sequence identity to the sequence of SEQ ID NO:37. In embodiments, the anticancer therapeutic antibody domain has at least 98% sequence identity to the sequence of SEQ ID NO:37. In embodiments, the anticancer therapeutic antibody domain is the sequence of SEQ ID NO:37.

In embodiments, the recombinant protein provided herein includes a first Fc domain including the sequence of SEQ ID NO:38, wherein the first Fc domain is covalently attached to a chemokine domain including the sequence of SEQ ID NO:1; a second Fc domain including the sequence of SEQ ID NO:40, wherein the second Fc domain is covalently attached to an anticancer therapeutic antibody domain including the sequence of SEQ ID NO:37; wherein the chemokine domain is covalently attached to the N-terminus of the first Fc domain by a peptide linker including the sequence of SEQ ID NO:39, and wherein the anticancer therapeutic antibody domain is covalently attached to the N-terminus of the second Fc domain by a peptide linker including the sequence of SEQ ID NO:39.

In embodiments, the recombinant protein provided herein includes a first Fc domain including the sequence of SEQ ID NO:40, wherein the first Fc domain is covalently attached to a chemokine domain including the sequence of SEQ ID NO:1; a second Fc domain including the sequence of SEQ ID NO:38, wherein the second Fc domain is covalently attached to an anticancer therapeutic antibody domain including the sequence of SEQ ID NO:37; wherein the chemokine domain is covalently attached to the N-terminus of the first Fc domain by a peptide linker including the sequence of SEQ ID NO:39, and wherein the anticancer therapeutic antibody domain is covalently attached to the N-terminus of the second Fc domain by a peptide linker including the sequence of SEQ ID NO:39.

### Nucleic Acid Compositions

The compositions provided herein include nucleic acid molecules encoding the recombinant proteins provided herein including embodiments thereof. The recombinant proteins provided herein encoded by the isolated nucleic acid are described in detail throughout this application (including the description above and in the examples section). Thus, in an aspect, a nucleic acid encoding the recombinant protein as provided herein including embodiments thereof is provided. In embodiments, the nucleic acid includes an expression cassette.

In embodiments, the nucleic acid includes a viral promoter or a tumor specific promoter. In embodiments, the nucleic acid includes a viral promoter. In embodiments, the nucleic acid includes a tumor specific promoter. The term “tumor specific promoter” refers to a promoter that has higher activity in a tumor cell as compared to a non-tumor cell. For example, a gene that is operably linked to a tumor specific promoter will be expressed in higher levels in the tumor cell as compared to a non-tumor cell as compared to a tumor cell. In embodiments, expression of a gene that is operably linked to a tumor specific promoter is increased by at least about 40% 50%, 60%, 70%, 80%, 90%, or 100% in a tumor cell compared to expression of the gene more in a non-tumor cell. In embodiments, expression of a gene that is operably linked to a tumor specific promoter is increased by at least about 40% in a tumor cell compared to expression of the gene more in a non-tumor cell. In embodiments, expression of a gene that is operably linked to a tumor specific promoter is increased by at least about 50%. In embodiments, expression of a gene that is operably linked to a tumor specific promoter is increased by at least about 60% in a tumor cell compared to expression of the gene more in a non-tumor cell. In embodiments, expression of a gene that is operably linked to a tumor specific promoter is increased by at least about 70% in a tumor cell compared to expression of the gene more in a non-tumor cell. In embodiments, expression of a gene that is operably linked to a tumor specific promoter is increased by at least about 80% in a tumor cell compared to expression of the gene more in a non-tumor cell. In embodiments, expression of a gene that is operably linked to a tumor specific promoter is increased by at least about 90% in a tumor cell compared to expression of the gene more in a non-tumor cell. In embodiments, expression of a gene that is operably linked to a tumor specific promoter is increased by at least about 100% in a tumor cell compared to expression of the gene more in a non-tumor cell.

In embodiments, the virus specific promoter is a herpes simplex virus (HSV) promoter. In embodiments, the HSV promoter is an immediate early (IE) promoter. In embodiments, the HSV IE promoter is an IE 4/5 promoter. In embodiments, the promoter includes the sequence of SEQ ID NO:6. In embodiments, the promoter is the sequence of SEQ ID NO:6.

In embodiments, the nucleic acid encodes a detectable protein. In embodiments, the detectable protein is GFP, EGFP or RFP. In embodiments, the detectable protein is GFP. In embodiments, the detectable protein is EGFP. In embodiments, the detectable protein is RFP.

### Virus Compositions

Provided herein, inter alia, is a oncolytic virus including a nucleic acid encoding a recombinant protein provided herein including embodiments thereof. The oncolytic virus selectively replicates in tumor cells (e.g. glioblastoma cells), thereby allowing expression of the recombinant protein in the tumor microenvironment. As described herein, the recombinant protein includes a chemokine domain, which is contemplated to increase trafficking of responsive cells (e.g. effector cells) to the tumor microenvironment. The recombinant protein further includes an anticancer therapeutic antibody domain, which binds to antigens expressed on cancer cells. Thus, the recombinant protein allows redirection of the chemokine domain to tumor cells. Thus, in an aspect is provided an oncolytic virus including a nucleic acid encoding a recombinant protein including: (a) a first Fc fusion protein including a first Fc domain covalently attached to a chemokine domain; and (b) a second Fc fusion protein including a second Fc domain covalently attached to an anticancer therapeutic antibody domain, wherein the first Fc domain is capable of binding to the second Fc domain.

In embodiments, the nucleic acid is exogenous to the oncolytic virus. In embodiments, the nucleic acid includes an expression cassette. In embodiments, the nucleic acid includes a modification which allows selective replication in tumor cells. For example, in embodiments, mutations or loss of viral ICP6 (encoding a virual ribonucleotide reductase function) gene may cause dependency on mammalian cells that express ribonucleotide reductase. In embodiments, the recombinant oncolytic virus does not include a nucleic acid encoding a functional ICP6 gene. In embodiments, the oncolytic virus does not include a nucleic acid encoding a 734.5 gene.

In embodiments, the first Fc domain and the second Fc domain are independently a first IgG Fc domain and a second IgG Fc domain. In embodiments, the first IgG Fc domain is a first IgG1 Fc domain and the second IgG Fc domain is a second IgG1 Fc domain.

In embodiments, the first IgG domain is a first IgG1 Fc domain, a first IgG2 Fc domain, a first IgG3 Fc domain, or a first IgG4 Fc domain. In embodiments, said second IgG domain is a second IgG1 Fc domain, a second IgG2 Fc domain, a second IgG3 Fc domain, or a second IgG4 Fc domain.

In embodiments, the virus is an adenovirus, a herpes simplex virus, a maraba virus, a measles virus, a Newcastle virus, a picornavirus, a reovirus, a vaccinia virus, a vesicular stomatitis virus, a rubeloa virus, or a myxoma virus. In embodiments, the virus is an adenovirus. In embodiments, the virus is a herpes simplex virus. In embodiments, the herpes simplex virus is herpes simplex virus 1. In embodiments, the herpes simplex virus is herpes simplex virus 2. In embodiments, the virus is a maraba virus. In embodiments, the virus is a measles virus. In embodiments, the virus is a Newcastle virus. In embodiments, the virus is a picornavirus. In embodiments, the virus is a reovirus. In embodiments, the virus is a vaccinia virus. In embodiments, the virus is a vesicular stomatitis virus. In embodiments, the virus is a rubeloa virus. In embodiments, the virus is myxoma virus.

In embodiments, the chemokine is a CC type chemokine domain, a CXC type chemokine domain, a C type chemokine domain, or a CX3C type chemokine domain. In embodiments, wherein the chemokine domain is a CC type chemokine domain. In embodiments, wherein the CC type chemokine domain is a CCL5 domain.

In embodiments, the CC type chemokine domain is CCL2, CCL3, CCL4, CCL5, CCL8, CCL9, CCL10, CCL11, CCL13, CCL14, CCL18, CCL19, CCL20, CCL21, CCL25, or CCL27. In embodiments, the CC type chemokine domain is CCL2. In embodiments, the CC type chemokine domain is CCL3. In embodiments, the CC type chemokine domain is CCL4. In embodiments, the CC type chemokine domain is CCL5. In embodiments, the CC type chemokine domain is CCL8. In embodiments, the CC type chemokine domain is CCL9. In embodiments, the CC type chemokine domain is CCL10. In embodiments, the CC type chemokine domain is CCL11. In embodiments, the CC type chemokine domain is CCL14. In embodiments, the CC type chemokine domain is CCL19. In embodiments, the CC type chemokine domain is CCL20. In embodiments, the CC type chemokine domain is CCL21. In embodiments, the CC type chemokine domain is CCL25. In embodiments, the CC type chemokine domain is CCL27.

In embodiments, the chemokine domain is a CXC type chemokine domain. In embodiments, the CXC type chemokine domain is CXCL8, CXCL10, CXCL12 or CXCL13. In embodiments, the CXC type chemokine domain is CXCL8. In embodiments, the CXC type chemokine domain is CXCL10. In embodiments, the CXC type chemokine domain is CXCL12. In embodiments, the CXC type chemokine domain is CXCL13.

In embodiments, the anticancer therapeutic antibody domain is a anti-EGFR antibody domain, an anti-Her2 antibody domain, an anti VEGF/VEGFR2 antibody domain, an anti-PDGFRα antibody domain, an anti-CD52 antibody domain, an anti-CD38 antibody domain, an anti-RANKL antibody domain, an anti-GD2 antibody domain, an anti-SLAMF7 antibody domain, an anti-CCR4 antibody domain, an anti-CD20 antibody domain, and an anti-CD19 antibody domain. In embodiments, the anticancer therapeutic antibody domain is an anti-EGFR antibody domain. In embodiments, the anticancer therapeutic antibody domain is an anti-Her2 antibody domain. In embodiments, the anticancer therapeutic antibody domain is an anti VEGF/VEGFR2 antibody domain. In embodiments, the anticancer therapeutic antibody domain is an anti-PDGFRα antibody domain. In embodiments, the anticancer therapeutic antibody domain is an anti-CD52 antibody domain. In embodiments, the anticancer therapeutic antibody domain is an anti-CD38 antibody domain. In embodiments, the anticancer therapeutic antibody domain is an anti-RANKL antibody domain. In embodiments, the anticancer therapeutic antibody domain is an anti-GD2 antibody domain. In embodiments, the anticancer therapeutic antibody domain is an anti-SLAMF7 antibody domain, an anti-CCR4 antibody domain. In embodiments, the anticancer therapeutic antibody domain is an anti-CD20 antibody domain. In embodiments, the anticancer therapeutic antibody domain is an anti-CD19 antibody domain.

In embodiments, the anticancer therapeutic antibody domain is an anti-EGFR antibody domain or an anti-Her2 antibody domain. In embodiments, the anticancer therapeutic antibody domain is an anti-EGFR antibody domain. In embodiments, the anti-EGFR antibody domain is a Fab domain or an scFv domain.

In embodiments, the nucleic acid encoding the recombinant protein includes a viral promoter or a tumor specific promoter. In embodiments, the viral promoter is a herpes simplex virus (HSV) promoter. In embodiments, the herpes simplex virus (HSV) promoter is an immediate early (IE) promoter. In embodiments, the HSV IE promoter is IE 4/5 promoter.

In an aspect, provided herein are recombinant oncolytic viruses including an expression cassette encoding a recombinant protein that includes a first Fc fusion protein including a first Fc domain covalently linked to a chemokine domain and a second Fc fusion protein including a second Fc domain covalently linked to an anticancer therapeutic antibody domain, where the first Fc domain is capable of binding to the second Fc domain.

In embodiments, the recombinant oncolytic viruses, including an expression cassette encoding a recombinant protein described herein, is selected from an adenovirus, a herpes simplex virus 1, a herpes simplex virus 2, a maraba virus, a measles virus, a Newcastle virus, a picornavirus, a reovirus, a vaccinia virus, a vesicular stomatitis virus, a rubeloa virus, and a myxoma virus. In embodiments, the recombinant oncolytic viruses, including an expression cassette encoding a recombinant protein described herein, is a herpes simplex virus. In embodiments, the recombinant herpes simplex oncolytic virus includes or does not include nucleic acid encoding a 734.5 gene. In embodiments, the recombinant herpes simplex oncolytic virus does not include nucleic acid encoding a functional ICP6 gene.

In embodiments, the recombinant oncolytic viruses, including an expression cassette encoding a recombinant protein described herein further include a nucleic acid encoding a cytokine gene, a chemokine gene, or a gene encoding an antibody or a fragment thereof (e.g. Fab domain, scFv), a suicide gene, or an shRNA.

In embodiments, provided herein are recombinant oncolytic viruses including an expression cassette encoding a recombinant protein including a first Fc fusion protein and a second Fc fusion protein. In embodiments of the recombinant oncolytic viruses herein, the first Fc fusion protein includes a first Fc domain. In embodiments, the first Fc domain is a first IgG Fc domain. In embodiments, the first Fc domain is a first IgG1 domain, a first IgG2 domain, a first IgG3 domain, or a first IgG4 domain. In embodiments, the first Fc domain is a first IgG1 domain. In embodiments, the first Fc domain is a first IgG2 domain. In embodiments, the first Fc domain is a first IgG3 domain. In embodiments, the first IgG domain is a first IgG4 domain.

In embodiments, provided herein are recombinant oncolytic viruses including an expression cassette encoding a recombinant protein including a first Fc fusion protein where the first Fc domain is covalently linked to a chemokine domain. In embodiments, the recombinant oncolytic viruses, including an expression cassette encoding a recombinant protein described herein, include a chemokine domain.

In embodiments, provided herein are recombinant oncolytic viruses including an expression cassette encoding a recombinant protein including a second Fc fusion protein. In embodiments, the second Fc fusion protein includes a second Fc domain. In embodiments, the second Fc domain is a second IgG Fc domain. In embodiments, the second Fc domain is a second IgG1 domain, a second IgG2 domain, a second IgG3 domain, or a second IgG4 domain. In embodiments, the second Fc domain is a second IgG1 domain. In embodiments, the second Fc domain is a second IgG2 domain. In embodiments, the second Fc domain is a second IgG3 domain. In embodiments, the second Fc domain is a second IgG4 domain.

In embodiments, provided herein are recombinant oncolytic viruses, including an expression cassette encoding a second Fc fusion protein including a second Fc domain covalently linked to an anticancer therapeutic antibody domain. In embodiments, the recombinant oncolytic viruses, including an expression cassette encoding a recombinant protein described herein, include an anticancer therapeutic antibody domain.

In embodiments of the recombinant oncolytic viruses including an expression cassette encoding a recombinant protein described herein, the anticancer therapeutic antibody domain is an anti-EGFR antibody domain. In embodiments of the recombinant oncolytic viruses including an expression cassette encoding a recombinant protein described herein, the anti-EGFR antibody domain is a cetuximab antibody domain.

In embodiments, the recombinant oncolytic viruses including an expression cassette encoding a recombinant protein where the first Fc domain is a first IgG1 domain and the second Fc domain is a second IgG1 domain. In embodiments, the recombinant oncolytic viruses including an expression cassette encoding recombinant proteins described herein include where the first Fc domain is a first IgG2 domain and the second Fc domain is a second IgG2 domain. In embodiments, the recombinant oncolytic viruses including an expression cassette encoding recombinant proteins described herein include where the first Fc domain is a first IgG3 domain and the second Fc domain is a second IgG3 domain. In embodiments, the recombinant oncolytic viruses including an expression cassette encoding recombinant proteins described herein include where the first Fc domain is a first IgG4 domain and the second Fc domain is a second IgG4 domain.

In embodiments of the recombinant oncolytic viruses including an expression cassette encoding a recombinant protein described herein, the nucleic acid encoding the recombinant protein is under the control of a viral or tumor specific promoter. In embodiments of the recombinant oncolytic viruses including an expression cassette encoding a recombinant protein described herein, the nucleic acid encoding the recombinant protein is under the control of a viral. In embodiments of the recombinant oncolytic viruses including an expression cassette encoding a recombinant protein described herein, the nucleic acid encoding the recombinant protein is under the control of a tumor specific promoter.

In embodiments of the recombinant oncolytic viruses including an expression cassette encoding a recombinant protein described herein, the viral promoter is a herpes simplex virus (HSV) promoter. In embodiments, the herpes simplex virus (HSV) promoter is an immediate early (IE) promoter. In embodiments, the HSV IE promoter is IE 4/5 promoter.

### Pharmaceutical Compositions

The compositions provided herein include the recombinant protein provided herein or the oncolytic virus provided herein. Thus, in an aspect is provided a pharmaceutical composition including the recombinant protein provided herein including embodiments thereof, and a pharmaceutically acceptable carrier.

In another aspect is provided a pharmaceutical composition including the oncolytic virus provided herein including embodiments thereof, and a pharmaceutically acceptable carrier.

The compositions provided herein further include a nucleic acid encoding the recombinant protein provided herein. Thus, in an aspect is provided a pharmaceutical composition including a nucleic acid encoding the recombinant protein provided herein including embodiments thereof, and a pharmaceutically acceptable carrier.

### Methods of Killing Cancer Cells

The compositions provided herein, including embodiments thereof, are further contemplated for killing cancer cells (e.g. glioblastoma cells). For example, the anticancer therapeutic antibody domain provided herein selectively targets cancer cells and the chemokine domain trafficks effector cells (e.g. NK cells, macrophages, T cells, etc.) to said cancer cells. Thus, the compositions provided herein may effectively kill tumor cells by ADCC and/or ADCD. Thus, in an aspect is provided a method of killing a cancer cell, the method including contacting the cancer cell with an oncolytic virus provided herein including embodiments thereof. In embodiments, the method includes contacting the cancer cell with an effective amount of the oncolytic virus provided herein including embodiments thereof.

In embodiments, the method further includes contacting the cancer cell with a plurality of immune cells. In embodiments, the immune cells include NK cells. In embodiments, said immune cells include macrophages. In embodiments, the cancer cell is in a subject having cancer.

In another aspect is provided a method of killing a cancer cell, the method including contacting the cancer cell with the recombinant protein provided herein including embodiments thereof. In embodiments, the method includes contacting the cancer cell with an effective amount of the recombinant protein provided herein including embodiments thereof.

In another aspect is provided a method of killing a cancer cell, the method including contacting the cancer cell with an effective amount of a nucleic acid encoding the recombinant protein provided herein including embodiments thereof. In embodiments, the nucleic acid includes a vector or a plasmid. In embodiments, the nucleic acid is delivered by a liposome. In embodiments, the nucleic acid provided herein can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

In embodiments, the nucleic acid is delivered by viral-based methods of transfection using any viral vector. Examples for viral vectors include, but are not limited to retroviral, adenoviral, lentiviral and adeno-associated viral vectors. In embodiments, the nucleic acid molecules are introduced into a cell using a retroviral vector following standard procedures well known in the art. Thus, in embodiments, the nucleic acid is delivered to a cell by a viral vector. In embodiments, the viral delivery system is not an oncolytic virus. In embodiments, the viral vector used for delivering the nucleic acid provided herein is capable of targeting a particular cell type either specifically or non-specifically. The vector may include a replication defective retrovirus, adenovirus or adeno-associated virus. Replication-incompetent viral vectors or replication-defective viral vectors refer to viral vectors that are capable of infecting their target cells and delivering their viral payload, but then fail to continue the typical lytic pathway that leads to cell lysis and death.

In embodiments, the methods provided herein including embodiments thereof further include contacting the cancer cell with a plurality of immune cells. In embodiments, the immune cells include NK cells. In embodiments, said immune cells include macrophages. In embodiments, the cancer cell is in a subject having cancer.

### Methods of Treatment

The compositions provided herein including embodiments thereof are contemplated as providing effective treatments for diseases (e.g. cancer). Thus, in an aspect is a method of treating or preventing cancer in a subject in need thereof, including administering to the subject a therapeutically effective amount of the oncolytic virus of provided herein including embodiments thereof, or a pharmaceutical composition provided herein including embodiments thereof.

In embodiments, the oncolytic virus is administered with at least 10² plaque forming units (Pfu)/kg. In embodiments, the oncolytic virus is administered with at least 10³ plaque forming units (Pfu)/kg. In embodiments, the oncolytic virus is administered with at least 10⁴ plaque forming units (Pfu)/kg. In embodiments, the oncolytic virus is administered with at least 10⁵ plaque forming units (Pfu)/kg. In embodiments, the oncolytic virus is administered with at least 10⁶ plaque forming units (Pfu)/kg. In embodiments, the oncolytic virus is administered with at least 10⁷ plaque forming units (Pfu)/kg. In embodiments, the oncolytic virus is administered with at least 108 plaque forming units (Pfu)/kg. In embodiments, the oncolytic virus is administered with at least 10⁹ plaque forming units (Pfu)/kg. In embodiments, the oncolytic virus is administered with at least 10¹⁰ plaque forming units (Pfu)/kg.

In embodiments, the oncolytic virus is administered at 10² plaque forming units (Pfu)/kg. In embodiments, the oncolytic virus is administered at 10³ plaque forming units (Pfu)/kg. In embodiments, the oncolytic virus is administered at 10⁴ plaque forming units (Pfu)/kg. In embodiments, the oncolytic virus is administered at 10⁵ plaque forming units (Pfu)/kg. In embodiments, the oncolytic virus is administered at 10⁶ plaque forming units (Pfu)/kg. In embodiments, the oncolytic virus is administered at 10⁷ plaque forming units (Pfu)/kg. In embodiments, the oncolytic virus is administered at 108 plaque forming units (Pfu)/kg. In embodiments, the oncolytic virus is administered at 10⁹ plaque forming units (Pfu)/kg. In embodiments, the oncolytic virus is administered at 10¹⁰ plaque forming units (Pfu)/kg.

In embodiments, the oncolytic virus is administered at about 10² plaque forming units (Pfu)/kg. In embodiments, the oncolytic virus is administered at 10² plaque forming units (Pfu)/kg. In embodiments, the oncolytic virus is administered at about 10³ plaque forming units (Pfu)/kg. In embodiments, the oncolytic virus is administered at 10³ plaque forming units (Pfu)/kg. In embodiments, the oncolytic virus is administered at about 4×10⁴ plaque forming units (Pfu)/kg. In embodiments, the oncolytic virus is administered at 4×10⁴ plaque forming units (Pfu)/kg. In embodiments, the oncolytic virus is administered at about 5×10⁴ plaque forming units (Pfu)/kg. In embodiments, the oncolytic virus is administered at 5×10⁴ plaque forming units (Pfu)/kg. In embodiments, the oncolytic virus is administered at about 10⁵ plaque forming units (Pfu)/kg. In embodiments, the oncolytic virus is administered at 10⁵ plaque forming units (Pfu)/kg.

In embodiments, the cancer is a chronic cancer (e.g. ovarian cancer, chronic leukemia, chronic lymphoma, metastatic cancer, etc.). A “chronic cancer” is used in accordance with its ordinary meaning in the art and refers to a cancer that is likely to recur after remission of the cancer. For example, symptoms of a cancer may be reduced and/or disappear, or cancer cells may not be detected at low levels before recurrence of the cancer.

In embodiments, the cancer is an inflammatory chronic cancer. In embodiments, the cancer is an EGFR expressing cancer. An EGFR expressing cancer refers to a cancer wherein the cancer cell expresses higher levels of EGFR compared to a non-cancer cell. In embodiments, the EGFR expressing cancer is glioblastoma, ovarian cancer, pancreatic cancer, leukemia, lymphoma, lung cancer, breast cancer, and brain metastatic tumor of a primary tumor. In embodiments, the EGFR expressing cancer is glioblastoma. In embodiments, the EGFR expressing cancer is ovarian cancer. In embodiments, the EGFR expressing cancer is pancreatic cancer. In embodiments, the EGFR expressing cancer is leukemia. In embodiments, the EGFR expressing cancer is lymphoma. In embodiments, the EGFR expressing cancer is lung cancer. In embodiments, the EGFR expressing cancer is breast cancer. In embodiments, the EGFR expressing cancer is brain metastatic tumor of a primary tumor.

In embodiments, the oncolytic virus or pharmaceutical composition is administered by intraperitoneal, intratumoral, intravenous, intrathecal, intrapleural, or intracavitary administration. In embodiments, the administration is by intraperitoneal, intratumoral, intravenous, intrathecal, intrapleural. or intracavitary administration. In embodiments, the administration is by intraperitoneal administration. In embodiments, the administration is by intratumoral administration. In embodiments, the administration is by intravenous administration. In embodiments, the administration is by intrathecal administration. In embodiments, the administration is by intrapleural administration. In embodiments, the administration is by intracavitary administration.

In embodiments, the method further includes administering to the subject an anticancer therapeutic. In embodiments, the anticancer therapeutic is administered prior to the onocolytic virus provided herein including embodiments thereof. In embodiments, the anticancer therapeutic is administered simultaneously with the onocolytic virus. In embodiments, the anticancer therapeutic is a DNA alkylating agent. In embodiments, the anticancer therapeutic is temzolomide, carmustine, bevacizumab, or lomustine. In embodiments, the anticancer therapeutic is temzolomide. In embodiments, the anticancer therapeutic is carmustine. In embodiments, the anticancer therapeutic is bevacizumab. In embodiments, the anticancer therapeutic is lomustine.

In embodiments, the oncolytic virus provided herein including embodiments thereof and the anticancer therapeutic have a synergistic effect. In embodiments, the anticancer therapeutic has a synergistic effect on the oncolytic virus. A “synergistic amount” as used herein refers to the sum of a first amount (e.g., an amount of the oncolytic virus or recombinant provided herein) and a second amount (e.g., an anticancer agent) that results in a synergistic effect (i.e. an effect greater than an additive effect). The terms “synergy”, “synergism”, “synergistic”, “synergistic effect”, “combined synergistic amount”, and “synergistic therapeutic effect” which are used herein interchangeably, refer to a measured effect of the compositions administered in combination, where the measured effect is greater than the sum of the individual effects of each of the compositions provided herein administered alone as a single agent. In embodiments, the measured effect is cancer cell death (e.g. by ADCC, etc).

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the oncolytic virus provided herein when used separately from the therapeutic agent (e.g. TMZ). In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the therapeutic agent (e.g. TMZ) when used separately from the oncolytic virus provided herein.

In an aspect is provided a method of treating or preventing cancer in a subject in need thereof, including administering to the subject a therapeutically effective amount of the recombinant protein provided herein including embodiments thereof. In embodiments, the cancer is a chronic cancer (e.g. ovarian cancer, chronic leukemia, chronic lymphoma, metastatic cancer, etc.). In embodiments, the cancer is an inflammatory chronic cancer. In embodiments, the cancer is an EGFR expressing cancer. In embodiments, the EGFR expressing cancer is glioblastoma, ovarian cancer, pancreatic cancer, leukemia, lymphoma, lung cancer, breast cancer, and brain metastatic tumor of a primary tumor.

In embodiments, the recombinant protein is administered by intraperitoneal, intratumoral, intravenous, intrathecal, intrapleural, or intracavitary administration. In embodiments, administering includes oral administration, administration as a suppository, topical contact, intravenous, systemic, intracavitary, intraventricular, parenteral, intraperitoneal, intramuscular, intralesional, intrathecal, intranasal or subcutaneous administration. In embodiments, administering includes by intratumoral administration. In embodiments, administering includes by systemic administration. In embodiments, administering includes by intracavitary administration.

In embodiments, the method further includes administering to the subject an anticancer therapeutic. In embodiments, the anticancer therapeutic is administered prior to the recombinant protein provided herein including embodiments thereof. In embodiments, the anticancer therapeutic is administered simultaneously with the recombinant protein. In embodiments, the anticancer therapeutic is temzolomide, carmustine, bevacizumab, or lomustine. In embodiments, the recombinant protein provided herein including embodiments thereof and the anticancer therapeutic have a synergistic effect.

In another aspect is provided a method of treating or preventing cancer in a subject in need thereof, including administering to the subject a therapeutically effective amount of a nucleic acid encoding the recombinant protein provided herein including embodiments thereof.

In embodiments, the cancer is a chronic cancer (e.g. ovarian cancer, chronic leukemia, chronic lymphoma, metastatic cancer, etc.). In embodiments, the cancer is an inflammatory chronic cancer. In embodiments, the cancer is an EGFR expressing cancer. In embodiments, the EGFR expressing cancer is glioblastoma, ovarian cancer, pancreatic cancer, leukemia, lymphoma, lung cancer, breast cancer, and brain metastatic tumor of a primary tumor.

In embodiments, the method further includes administering to the subject an anticancer therapeutic. In embodiments, the anticancer therapeutic is administered prior to the nucleic acid provided herein including embodiments thereof. In embodiments, the anticancer therapeutic is administered simultaneously with the nucleic acid. In embodiments, the anticancer therapeutic is temzolomide, carmustine, bevacizumab, or lomustine. In embodiments, the nucleic acid provided herein including embodiments thereof and the anticancer therapeutic have a synergistic effect.

### Methods of Inducing an Immune Response

In another aspect is provided a method of inducing an immune response in a subject in need thereof, the method including administering to the subject an effective amount of the oncolytic virus provided herein or the pharmaceutical composition provided herein. In embodiments, virus is administered at a dose from about 10² plaque forming units (Pfu)/kg to about 10¹⁰ Pfu/kg.

In embodiments, inducing an immune response includes activating adaptive immune cells. In embodiments, the adaptive immune cells include T cells. In embodiments, inducing an immune response includes activating innate immune cells. In embodiments, innate immune cells include natural killer (NK) cells and macrophages. In embodiments, activating innate immune cells is measured as increased expression of CD69 by an NK cell in a subject who has been administered an oncolytic virus, recombinant protein or nucleic acid provided herein compared to an NK cell in a subject who has not been administered an oncolytic virus, recombinant protein or nucleic acid provided herein including embodiments thereof. For example, an NK cell in a subject who has been administered an oncolytic virus, recombinant protein, or nucleic acid provided herein may express 10%, 20%, 30%, 40%, 50%, 60%, 70%, 80%, 90% or 100% higher levels of CD69 than an NK cell in a subject who has not been administered an oncolytic virus, recombinant protein, or nucleic acid provided herein including embodiments thereof. In embodiments, activating innate immune cells is measured by increased cytotoxicity of an NK cell. For example, increased NK cell cytotoxicity may be measured as increased cancer cell death in a subject who has been administered an oncolytic virus, recombinant protein or nucleic acid provided herein as compared to NK cell cytoxicity in a subject who has not been administered an oncolytic virus, recombinant protein or nucleic acid provided herein. Cancer cell death may be measured in a subject or in vitro (e.g. in a cell culture including the oncolytic virus, recombinant protein or nucleic acid provided herein including embodiments thereof). Cancer cell death may be measured in a variety of methods well known in the art including but not limited to measuring annexin V binding, flow cytometry, caspase activation and detection, mitochondrial membrane potential-dependent cyes and cytochrome C release assays. In embodiments, activating immune cells may be measured as decreased tumor size in a subject who has been administered an oncolytic virus, recombinant protein or nucleic acid provided herein compared to a subject who has not been administered an oncolytic virus, recombinant protein or nucleic acid provided herein.

In embodiments, innate immune cells include macrophages. In embodiments, inducing an immune response includes increasing macrophage mediated antibody-dependent cellular phagocytosis. In embodiments, increased macrophage ADCP can be measured by increased expression of IL-1B, IL-6, IL-12, or INOS. In embodiments, increased macrophage mediated ADCP is measured as increased expression of IL-1B, IL-6, IL-12, or INOS by a macrophage in a subject who has been administered an oncolytic virus, recombinant protein or nucleic acid provided herein compared to a macrophage in a subject who has not been administered an oncolytic virus, recombinant protein or nucleic acid provided herein including embodiments thereof. In embodiments, increased macrophage mediated ADCP is measured as increased expression of IL-1B. In embodiments, increased macrophage mediated ADCP is measured as increased expression of IL-6. In embodiments, increased macrophage mediated ADCP is measured as increased expression of IL-12. In embodiments, increased macrophage mediated ADCP is measured as increased expression of INOS.

In embodiments, inducing an immune response includes inducing migration of effector cells towards a target (e.g. target cancer cells). In embodiments, the effector cells are T cells, NK cells or macrophages. Inducing migration refers to directional movement of effector cells towards the target cell. For example, effector cells may migrate towards a cancer cell bound to a recombinant protein provided herein including embodiments thereof. Effector cells may migrate towards a recombinant protein provided herein including embodiments thereof. Effector cells may migrate towards an oncolytic virus provided herein including embodiments thereof.

In embodiments, inducing an immune response may be in vitro. For example, effector cells (T cells, macrophages, NK cells) in a culture may exert cytoxic effects or cell killing as described herein, when in the presence of a recombinant protein or oncolytic virus provided herein including embodiments thereof.

In another aspect is provided a method of inducing an immune response in a subject in need thereof, the method including administering to the subject an effective amount of the recombinant protein provided herein including embodiments thereof. In embodiments, inducing an immune response includes activating adaptive immune cells. In embodiments, inducing an immune response includes activating innate immune cells. In embodiments, including an immune response includes increased cancer cell death.

In another aspect is provided a method of inducing an immune response in a subject in need thereof, the method including administering to the subject an effective amount of a nucleic acid encoding a recombinant protein provided herein including embodiments thereof.

### EMBODIMENTS

Embodiment P1. A recombinant protein comprising: a) a first Fc fusion protein comprising a first Fc domain covalently linked to a chemokine domain; and b) a second Fc fusion protein comprising a second Fc domain covalently linked to an anticancer therapeutic antibody domain, wherein the first Fc domain is capable of binding to the second Fc domain.

Embodiment P2. The recombinant protein of embodiment 1, wherein the first Fc domain is a first IgG Fc domain and/or the second Fc domain is a second IgG Fc domain.

Embodiment P3. The recombinant protein of embodiment 2, wherein the first IgG domain is a first IgG1 domain, a first IgG2 domain, a first IgG3 domain, or a first IgG4 domain; and/or the second IgG domain is a second IgG1 domain, a second IgG2 domain, a second IgG3 domain, or a second IgG4 domain.

Embodiment P4. The recombinant protein of embodiment 3, wherein the first IgG domain is a first IgG1 domain and the second IgG domain is a second IgG1 domain.

Embodiment P5. The recombinant protein of any one of embodiments 1-4, wherein the chemokine domain is selected from a CC type chemokine domain, a CXC type chemokine domain, a C type chemokine domain, and a CX3C type chemokine domain.

Embodiment P6. The recombinant protein of any one of embodiments 1-5, wherein the chemokine domain is a CC type chemokine domain.

Embodiment P7. The recombinant protein of any one of embodiments 1-6, wherein the CC type chemokine domain is a CCL5 domain.

Embodiment P8. The recombinant protein of any one of embodiments 1-7, wherein the anticancer therapeutic antibody domain is selected from an anti-EGFR antibody domain and an anti-Her2 antibody domain.

Embodiment P9. The recombinant protein of any one of embodiments 1-8, wherein the anticancer therapeutic antibody domain is an anti-EGFR antibody domain.

Embodiment P10. The recombinant protein of embodiment 9, wherein the anti-EGFR antibody domain is a cetuximab antibody domain.

Embodiment P11. A nucleic acid encoding the recombinant protein of any one of embodiments 1-10.

Embodiment P12. A recombinant oncolytic virus comprising an expression cassette encoding a recombinant protein comprising: a) a first Fc fusion protein comprising a first Fc domain covalently linked to a chemokine domain; and b) a second Fc fusion protein comprising a second Fc domain covalently linked to an anticancer therapeutic antibody domain, wherein the first Fc domain is capable of binding to the second Fc domain.

Embodiment P13. The recombinant oncolytic virus of embodiment 12, wherein the first Fc domain is a first IgG Fc domain and/or the second Fc domain is a second IgG Fc domain.

Embodiment P14. The recombinant oncolytic virus of embodiment 13, wherein the first IgG domain is a first IgG1 domain, a first IgG2 domain, a first IgG3 domain, or a first IgG4 domain; and/or the second IgG domain is a second IgG1 domain, a second IgG2 domain, a second IgG3 domain, or a second IgG4 domain.

Embodiment P15. The recombinant oncolytic virus of embodiment 14, wherein the first IgG domain is a first IgG1 domain and the second IgG domain is a second IgG1 domain.

Embodiment P16. The recombinant oncolytic virus of any one of embodiments 12-15, wherein the virus is selected from an adenovirus, a herpes simplex virus, a maraba virus, a measles virus, a Newcastle virus, a picornavirus, a reovirus, a vaccinia virus, a vesicular stomatitis virus, a rubeloa virus, and a myxoma virus.

Embodiment P17. The recombinant oncolytic virus of any one of embodiments 12-16, wherein the oncolytic virus is a herpes simplex virus.

Embodiment P18. The recombinant oncolytic virus of any one of embodiments 12-16, wherein the oncolytic virus is a herpes simplex virus.

Embodiment P19. The recombinant oncolytic virus of any one of embodiments 12-18, wherein the recombinant oncolytic virus does not comprise nucleic acid encoding a functional ICP6 gene.

Embodiment P20. The recombinant oncolytic virus of any one of embodiments 12-19, wherein the chemokine is selected from a CC type chemokine domain, a CXC type chemokine domain, a C type chemokine domain, and a CX3C type chemokine domain.

Embodiment P21. The recombinant oncolytic virus of any one of embodiments 12-20, wherein the chemokine domain is a CC type chemokine domain.

Embodiment P22. The recombinant oncolytic virus of any one of embodiments 12-21, wherein the CC type chemokine domain is a CCL5 domain.

Embodiment P23. The recombinant oncolytic virus of any one of embodiments 12-22, wherein the anticancer therapeutic antibody domain is selected from an anti-EGFR antibody domain and an anti-Her2 antibody domain.

Embodiment P24. The recombinant oncolytic virus of any one of embodiments 12-23, wherein the anticancer therapeutic antibody domain is an anti-EGFR antibody domain.

Embodiment P25. The recombinant oncolytic virus of any one of embodiments 12-24, wherein the anti-EGFR antibody domain is a cetuximab antibody domain.

Embodiment P26. The recombinant oncolytic virus of any of embodiments 12-25, wherein the nucleic acid encoding the recombinant protein is under the control of a viral or tumor specific promoter.

Embodiment P27. The recombinant oncolytic virus of any of embodiments 12-26, wherein the viral promoter is a herpes simplex virus (HSV) promoter.

Embodiment P28. The recombinant oncolytic virus of embodiment 27, wherein the herpes simplex virus (HSV) promoter is an immediate early (IE) promoter.

Embodiment P29. The recombinant oncolytic virus of embodiment 28, wherein the HSV IE promoter is IE 4/5 promoter.

Embodiment P30. A pharmaceutical composition comprising: the recombinant oncolytic virus of any one of embodiments 12-29, and a pharmaceutically acceptable carrier.

Embodiment P31. A method for killing tumor cells in a subject comprising administering to a subject an effective amount of the recombinant oncolytic virus of any of embodiments 12-29.

Embodiment P32. A method of treating a subject having cancer comprising administering to the subject an effective amount of the pharmaceutical composition of embodiment 30 or the recombinant oncolytic virus of any of embodiments 12-29.

Embodiment P33. The method of embodiment 32, wherein the cancer is a chronic cancer.

Embodiment P34. The method of any one of embodiments 32-33, wherein the cancer is an inflammatory chronic cancer.

Embodiment P35. The method of embodiment 34, wherein the cancer is an EGFR expressing cancer.

Embodiment P36. The method of embodiment 35, wherein the EGFR expressing cancer is selected from glioblastoma, ovarian cancer, pancreatic cancer, leukemia, lymphoma, lung cancer, breast cancer, and brain metastatic tumor of an primary tumor.

Embodiment P37. The method of any of embodiments 31-36, wherein administering is by intratumoral, systemic, or intracavitary administration.

Embodiment P38. A method for immune modulation in a subject, comprising administering an effective amount of the recombinant oncolytic virus of any of embodiments 12-29 or the pharmaceutical composition of embodiment 30.

Embodiment P39. The method of embodiment 38, wherein immune modulation comprises activating innate immune and/or adaptive immune cells.

Embodiment P40. The method of embodiment 39, wherein immune modulation comprises activating innate immune cells.

Embodiment P41. The method of embodiment 40, wherein the innate immune cells are natural killer (NK) cells.

Embodiment P42. The method of embodiment 41, wherein activating NK cells is measured as induction of cell-mediated antibody-dependent cellular cytotoxicity.

Embodiment P43. The method of embodiment 42, wherein activating NK cells is measured as expression of CD69 on NK cells.

Embodiment P44. The method of any of embodiments 40-43, wherein activating NK cells is measured as increased NK cell cytotoxicity.

Embodiment P45. The method of any one of embodiments 31-44, wherein immune modulation comprises enhancing macrophage mediated antibody-dependent cellular cytotoxicity.

Embodiment P46. The method of embodiment 45, wherein enhancing macrophage mediated antibody-dependent cellular cytotoxicity is measured as expression of IL-1B, IL-6, IL-12, and INOS.

Embodiment P47. The method of any one of embodiments 31-46, wherein immune modulation comprises activating adaptive immune cells.

Embodiment P48. The method of embodiment 47, wherein the adaptive immune cells are T cells.

### EMBODIMENTS

Embodiment 1. A recombinant protein comprising: a) a first Fc fusion protein comprising a first Fc domain covalently attached to a chemokine domain; and b) a second Fc fusion protein comprising a second Fc domain covalently attached to an anticancer therapeutic antibody domain, wherein the first Fc domain is capable of binding to the second Fc domain.

Embodiment 2. The recombinant protein of embodiment 1, wherein the first Fc domain and the second Fc domain are independently a first IgG Fc domain and a second IgG Fc domain.

Embodiment 3. The recombinant protein of embodiment 1, wherein the first IgG Fc domain is a first IgG1 Fc domain and the second IgG Fc domain is a second IgG1 Fc domain.

Embodiment 4. The recombinant protein of embodiment 2 or 3, wherein the first IgG Fc domain is a first IgG1 Fc domain, a first IgG2 Fc domain, a first IgG3 Fc domain, or a first IgG4 Fc domain.

Embodiment 5. The recombinant protein of any one of embodiments 2-4, wherein said second IgG Fc domain is a second IgG1 Fc domain, a second IgG2 Fc domain, a second IgG3 Fc domain, or a second IgG4 Fc domain.

Embodiment 6. The recombinant protein of any one of embodiments 1-5, wherein said first Fc domain second Fc domain comprises a cysteine at a position corresponding to position 349 of SEQ ID NO:7, a serine at a position corresponding to position 366 of SEQ ID NO:7, an alanine at a position corresponding to position 368 of SEQ ID NO:7, a valine at a position corresponding to position 407 of SEQ ID NO:7, and a lysine at a position corresponding to position 405 of SEQ ID NO:7.

Embodiment 7. The recombinant protein of any one of embodiments 1-6, wherein said second Fc domain comprises a cysteine at a position corresponding to position 349 of SEQ ID NO:7, a serine at a position corresponding to position 366 of SEQ ID NO:7, an alanine at a position corresponding to position 368 of SEQ ID NO:7, a valine at a position corresponding to position 407 of SEQ ID NO:7, and a lysine at a position corresponding to position 405 of SEQ ID NO:7.

Embodiment 8. The recombinant protein of any one of embodiments 1-5, wherein said first Fc domain comprises a cysteine at a position corresponding to position 354 of SEQ ID NO:7, a tryptophan at a position corresponding to position 366 of SEQ ID NO:7, and/or an alanine at a position corresponding to position 409 of SEQ ID NO:7.

Embodiment 9. The recombinant protein of any one of embodiments 1-5 or 8, wherein said second Fc domain comprises a cysteine at a position corresponding to position 354 of SEQ ID NO:7, a tryptophan at a position corresponding to position 366 of SEQ ID NO:7, and/or an alanine at a position corresponding to position 409 of SEQ ID NO:7.

Embodiment 10. The recombinant protein of any one of embodiments 1-9, wherein the chemokine domain is a CC type chemokine domain, a CXC type chemokine domain, a C type chemokine domain, or a CX3C type chemokine domain.

Embodiment 11. The recombinant protein of embodiment 10, wherein the chemokine domain is a CC type chemokine domain.

Embodiment 12. The recombinant protein of embodiment 11, wherein the CC type chemokine domain is a CCL5 domain.

Embodiment 13. The recombinant protein of embodiment 12, wherein the CCL5 domain has at least 80% sequence identity to the sequence of SEQ ID NO:1.

Embodiment 14. The recombinant protein of any one of embodiments 1-13, wherein the anticancer therapeutic antibody domain is an anti-EGFR antibody domain or an anti-Her2 antibody domain.

Embodiment 15. The recombinant protein of any one of embodiments 1-14, wherein the anticancer therapeutic antibody domain is an anti-EGFR antibody domain.

Embodiment 16. The recombinant protein of embodiment 15, wherein the anti-EGFR antibody domain is a Fab domain or an scFv.

Embodiment 17. A nucleic acid encoding the recombinant protein of any one of embodiments 1-16.

Embodiment 18. A oncolytic virus comprising a nucleic acid encoding a recombinant protein comprising: a) a first Fc fusion protein comprising a first Fc domain covalently attached to a chemokine domain; and b) a second Fc fusion protein comprising a second Fc domain covalently attached to an anticancer therapeutic antibody domain, wherein the first Fc domain is capable of binding to the second Fc domain.

Embodiment 19. The oncolytic virus of embodiment 18, wherein said nucleic acid comprises an expression cassette.

Embodiment 20. The oncolytic virus of embodiment 18 or 19, wherein said first Fc domain and said second Fc domain are independently a first IgG Fc domain and a second IgG Fc domain.

Embodiment 21. The oncolytic virus of any one of embodiments 18-20, wherein the first IgG Fc domain is a first IgG1 Fc domain and the second IgG Fc domain is a second IgG1 Fc domain.

Embodiment 22. The oncolytic virus of embodiment 20 or 21, wherein the first IgG domain is a first IgG1 Fc domain, a first IgG2 Fc domain, a first IgG3 Fc domain, or a first IgG4 Fc domain.

Embodiment 23. The oncolytic virus of any one of embodiments 20-22, wherein said second IgG domain is a second IgG1 Fc domain, a second IgG2 Fc domain, a second IgG3 Fc domain, or a second IgG4 Fc domain.

Embodiment 24. The oncolytic virus of any one of embodiments 18-23, wherein the oncolytic virus is an adenovirus, a herpes simplex virus, a maraba virus, a measles virus, a Newcastle virus, a picornavirus, a reovirus, a vaccinia virus, a vesicular stomatitis virus, a rubeloa virus, or a myxoma virus.

Embodiment 25. The oncolytic virus of any one of embodiments 18-24, wherein the oncolytic virus is a herpes simplex virus.

Embodiment 26. The oncolytic virus of embodiment 25, wherein the oncolytic virus does not comprise a nucleic acid encoding a 734.5 gene.

Embodiment 27. The oncolytic virus of any one of embodiments 18-26, wherein the oncolytic virus does not comprise a nucleic acid encoding a functional ICP6 gene.

Embodiment 28. The oncolytic virus of any one of embodiments 18-27, wherein the chemokine is a CC type chemokine domain, a CXC type chemokine domain, a C type chemokine domain, or a CX3C type chemokine domain.

Embodiment 29. The oncolytic virus of embodiment 28, wherein the chemokine domain is a CC type chemokine domain.

Embodiment 30. The oncolytic virus of embodiment 29, wherein the CC type chemokine domain is a CCL5 domain.

Embodiment 31. The oncolytic virus of any one of embodiments 18-30, wherein the anticancer therapeutic antibody domain is an anti-EGFR antibody domain or an anti-Her2 antibody domain.

Embodiment 32. The oncolytic virus of embodiment 31, wherein the anticancer therapeutic antibody domain is an anti-EGFR antibody domain.

Embodiment 33. The oncolytic virus of embodiment 32, wherein the anti-EGFR antibody domain is a Fab domain or an scFv domain.

Embodiment 34. The oncolytic virus of any of embodiments 18-33, wherein the nucleic acid encoding the recombinant protein comprises a viral promoter or a tumor specific promoter.

Embodiment 35. The oncolytic virus of embodiment 34, wherein the viral promoter is a herpes simplex virus (HSV) promoter.

Embodiment 36. The oncolytic virus of embodiment 35, wherein the herpes simplex virus (HSV) promoter is an immediate early (IE) promoter.

Embodiment 37. The oncolytic virus of embodiment 36, wherein the HSV IE promoter is IE 4/5 promoter.

Embodiment 38. A pharmaceutical composition comprising the oncolytic virus of any one of embodiments 18-37, and a pharmaceutically acceptable excipient.

Embodiment 39. A method of killing a cancer cell, said method comprising contacting said cancer cell with the oncolytic virus of any one of embodiments 18-37.

Embodiment 40. The method of embodiment 39, further comprising contacting said cancer cell with a plurality of immune cells.

Embodiment 41. The method of embodiment 40, wherein said immune cells comprise NK cells.

Embodiment 42. The method of embodiment 40 or 41, wherein said immune cell comprise macrophages.

Embodiment 43. The method of embodiments 39-42, wherein said cancer cell is in a subject having cancer.

Embodiment 44. A method of treating or preventing cancer in a subject in need thereof, comprising administering to the subject a therapeutically effective amount of the oncolytic virus of any one of embodiments 18-37 or the pharmaceutical composition of embodiment 38.

Embodiment 45. The method of embodiment 44, wherein said virus is administered at a dose from about 10² plaque forming units (Pfu)/kg to about 10¹⁰ Pfu/kg.

Embodiment 46. The method of embodiment 44 or 45, wherein said cancer is a chronic cancer.

Embodiment 47. The method of embodiment 46, wherein said cancer is an inflammatory chronic cancer.

Embodiment 48. The method of any one of embodiments 44-47, wherein said cancer is a HER-2 or an EGFR expressing cancer.

Embodiment 49. The method of embodiment 48, wherein said EGFR expressing cancer is glioblastoma, ovarian cancer, pancreatic cancer, leukemia, lymphoma, lung cancer, breast cancer, or a brain metastatic tumor of an primary tumor.

Embodiment 50. The method of any one of embodiments 39-49, wherein said oncolytic virus or pharmaceutical composition is administered by intraperitoneal, intratumoral, intravenous, intrathecal, intrapleural. or intracavitary administration.

Embodiment 51. The method of any one of embodiments 39-50, further comprising administering an anticancer therapeutic.

Embodiment 52. A method of inducing an immune response in a subject in need thereof, said method comprising administering to said subject an effective amount of the oncolytic virus of any of embodiments 18-37 or the pharmaceutical composition of embodiment 38.

Embodiment 53. The method of embodiment 52, wherein said virus is administered at a dose from about 10² plaque forming units (Pfu)/kg to about 10¹⁰ Pfu/kg.

Embodiment 54. The method of embodiment 52 or 53, wherein inducing an immune response comprises activating adaptive immune cells.

Embodiment 55. The method of embodiment 53 or 54, wherein inducing an immune response comprises activating innate immune cells.

Embodiment 56. The method of embodiment 55, wherein said innate immune cells comprise natural killer (NK) cells and macrophages.

### EXAMPLES

### Introduction to Exemplary Experiments

Chemokines such as CCL5 regulate immune cell trafficking in the tumor microenvironment (TME) and govern tumor development, making them promising targets for cancer therapy; however, a short half-life and toxic off-target effects limit their application. Oncolytic virus (OV) therapies are becoming attractive therapeutic agents since the OV talimogene laherparepvec (T-VEC) received FDA approval for melanoma. In this study, we generated a novel oncolytic herpes simplex virus (oHSV) expressing a secretable single-chain variable fragment (scFv) of the epidermal growth factor receptor (EGFR) antibody cetuximab linked to CCL5 by an Fc knob-into-hole system that produces heterodimers (OV-Cmab-CCL5) and prevents homodimers. OV-Cmab-CCL5 allows continuous production of CCL5 in the TME as it is redirected to EGFR⁺ glioblastoma (GBM) tumor cells. In vitro, Cmab-CCL5 released by oHSV-infected GBM cells enhanced migration and activation of natural killer (NK) cells, macrophages, and T cells, and caused strong antibody-dependent cellular cytotoxicity (ADCC) by NK cells and antibody-dependent cellular phagocytosis (ADCP) by macrophages against EGFR⁺ GBM cells. In mouse models of GBM, OV-Cmab-CCL5 reduced tumor size, prolonged survival, and increased the appearance of intratumoral NK cells, macrophages, and T cells. Depletion of NK cells, macrophages, or T cells reduced the extended survival effects of OV-Cmab-CCL5, indicating that immune cell recruitment and activation contribute to OV-Cmab-CCL5's antitumor mechanism of action. Collectively, our data demonstrate that OV-Cmab-CCL5 provides a promising approach for improving OV therapy of solid tumors by enhancing the recruitment and activation of cytolytic lymphocytes and macrophages into the TME.

### Example 1: Experimental Design

Oncolytic viruses (OVs) are genetically engineered to selectively replicate in tumor cells and lyse them, and to enhance immune stimulation. OVs derived from engineered herpes simplex virus type 1 (oHSV) have been in clinical trials (11). Most recently, Friedman and colleagues reported that G207 oHSV showed a safe and strong effect on treating pediatric high-grade glioma (12). The success of oHSV-derived therapeutics is thought to depend both on the oncolytic destruction of tumor cells and the activation of antitumor immune responses, which can potentially lead to long-term cancer remission. In our previous studies, we also found that oHSV treatment dramatically increases the ability of immune cells to infiltrate tumors and destroy tumor cells (13-15). We thus reasoned that altering oHSV to enhance immune cell infiltration or increase the specificity of immune infiltration towards tumor cells would allow more robust tumor eradication.

Bispecific antibodies or fusion proteins are engineered to connect two receptor-binding functions into a single molecule. This “two-target” functionality can interfere with multiple surface receptors or ligands associated with tumors or other pathological processes (16). Various bispecific antibodies or fusion proteins are in clinical development or are already approved for cancer therapy (17,18). Thus, we engineered an oHSV encoding a bispecific fusion protein containing cetuximab, an antibody inhibitor of the epidermal growth factor receptor (EGFR), which can bind to both wild-type EGFR and the mutant EGFRvIII, and CCL5 (OV-Cmab-CCL5). Our construct combines chemokine function and antibody function together to improve GBM therapy. This novel OV not only targets the delivery of CCL5 to the TME, but also functions as an antibody by binding to heterogenous GBM cells to activate Fc-receptor-mediated NK cell antibody-dependent cellular cytotoxicity (ADCC) and macrophage antibody-dependent cellular phagocytosis (ADCP). In this study, we tested the OV-Cmab-CCL5 construct for the ability to activate antitumor immunity in vitro and in vivo, including an assessment of its ability to prolong survival in vivo.

### Example 2: OV-Cmab-hCCL5-Infected Glioblastoma Cells Secrete a Bispecific Fusion Protein Targeting Both the Oncogenic EGFR Receptor and the CCL5 Receptor

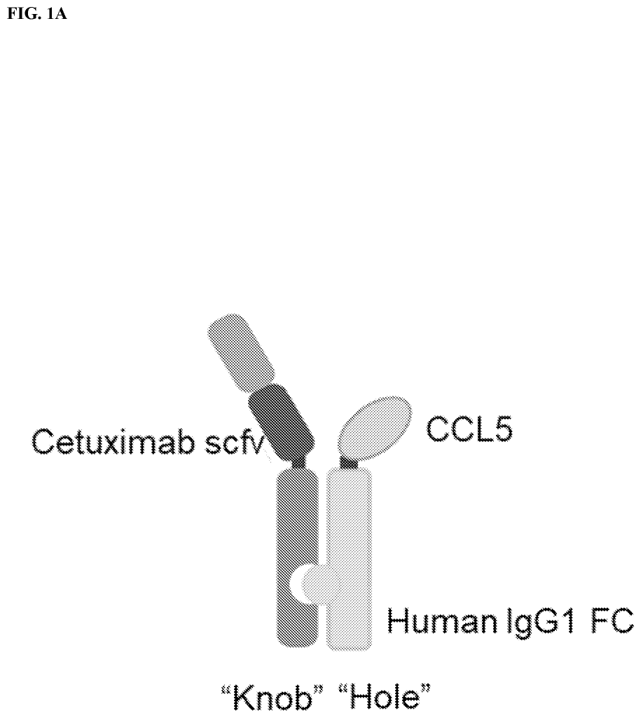

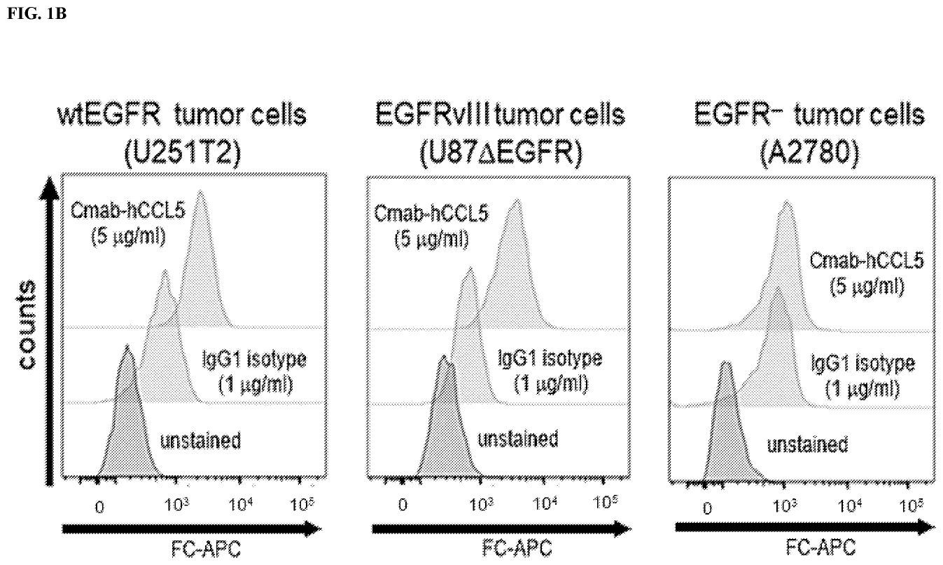

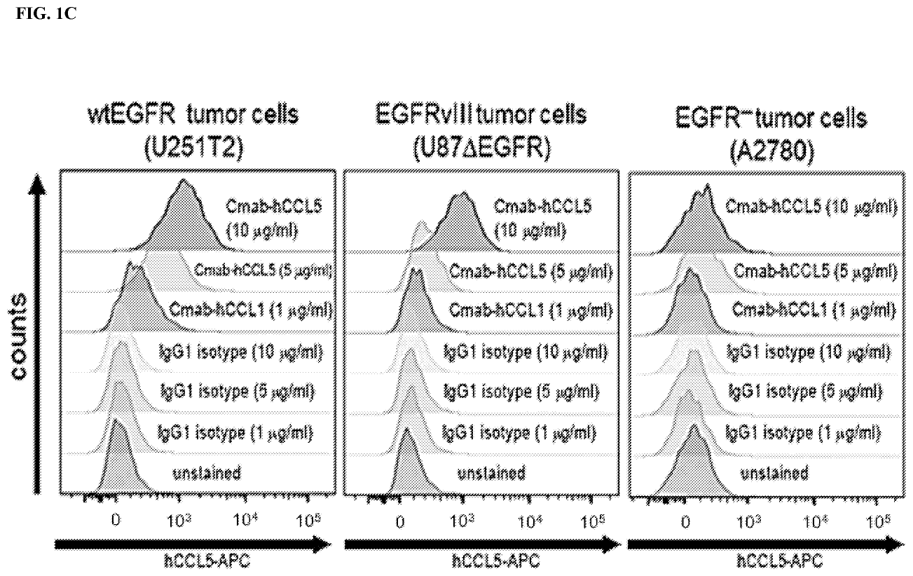

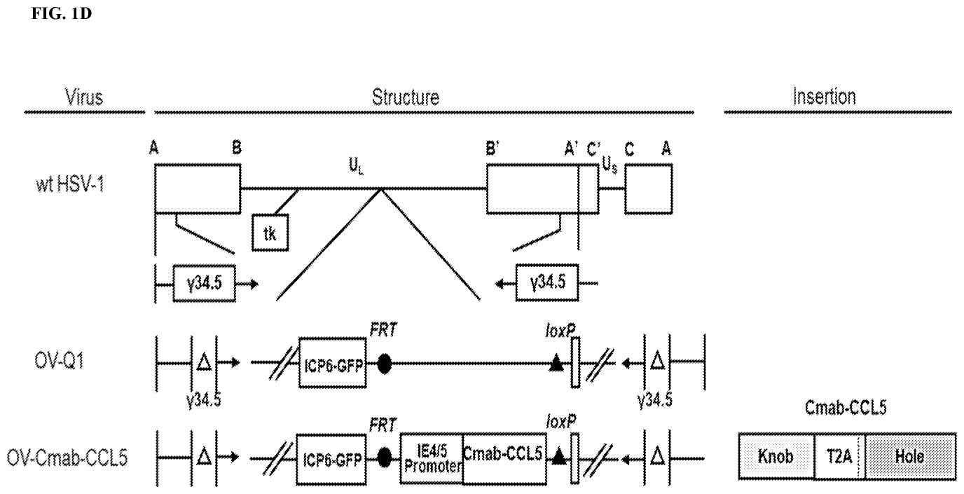

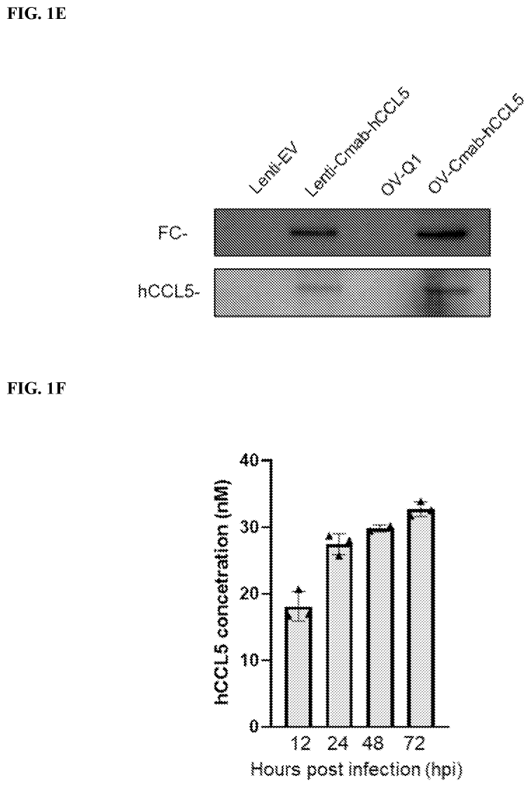

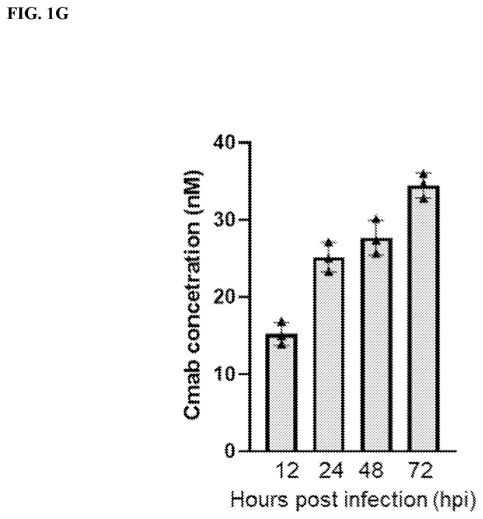

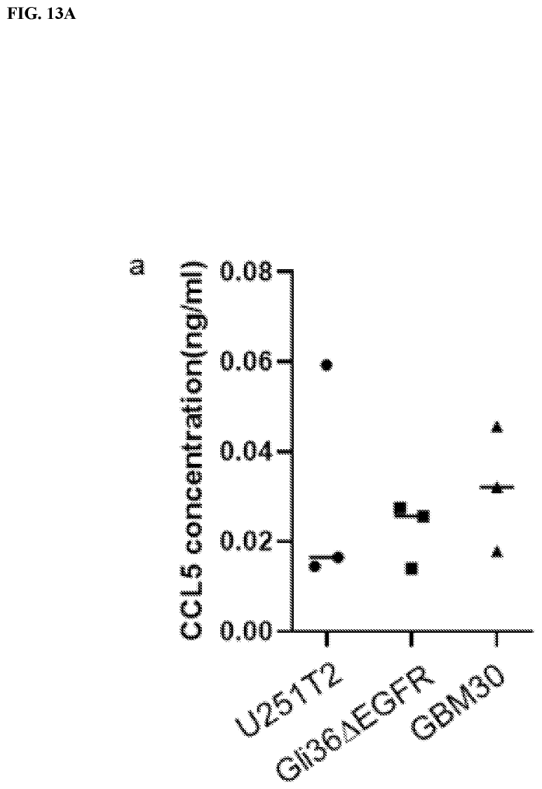

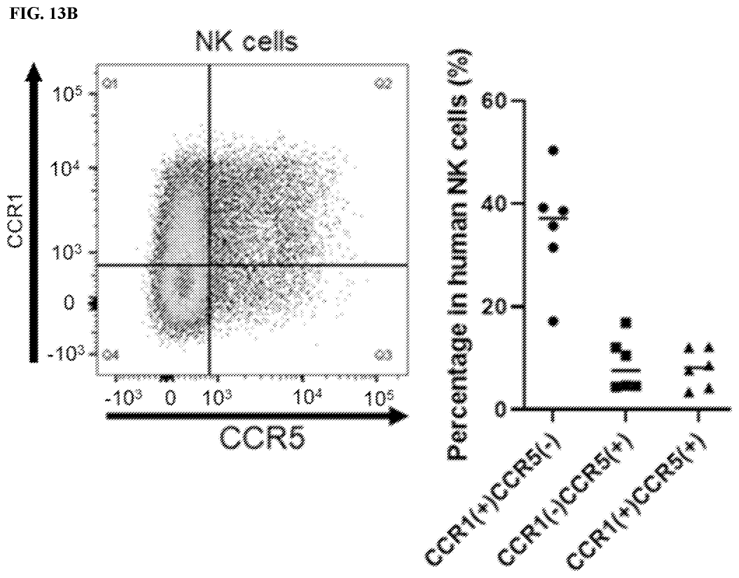

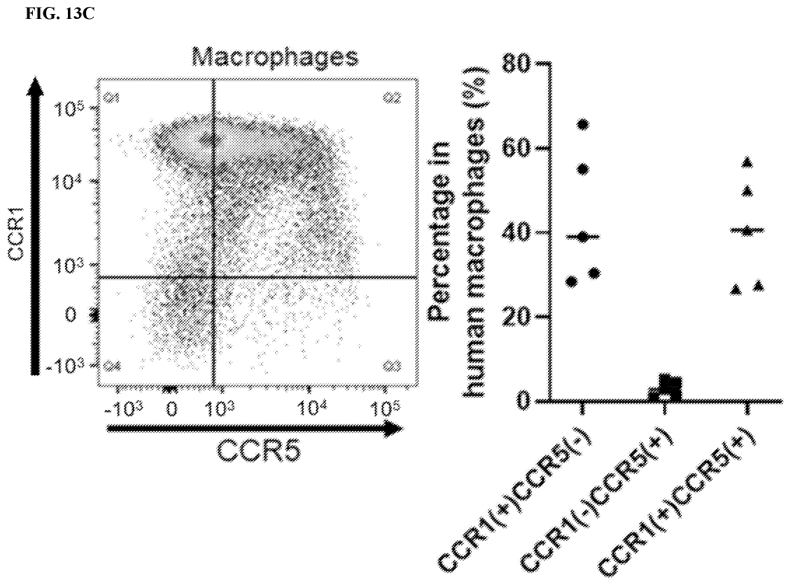

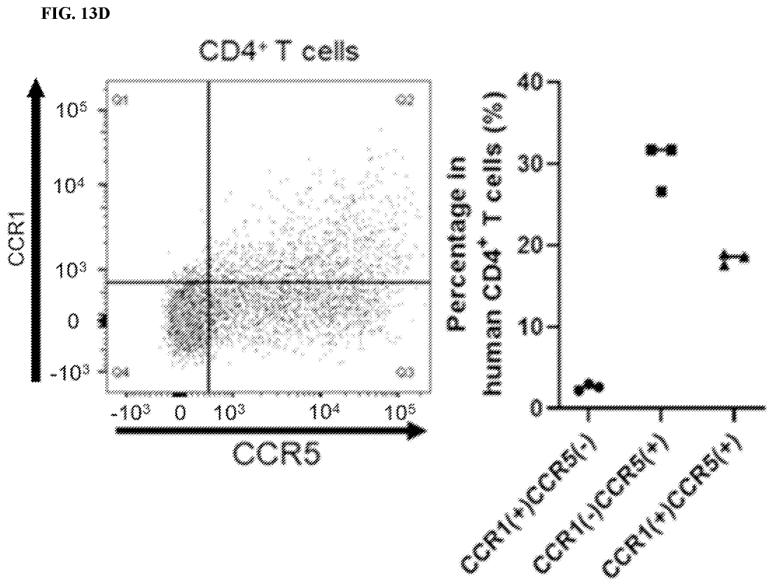

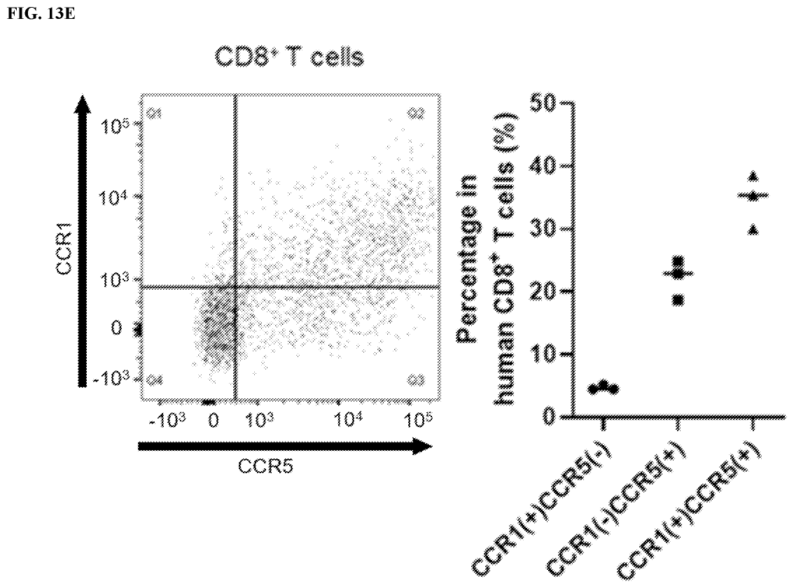

CCL5 is often epigenetically silenced in tumor cells (10). Therefore, as expected, we found that CCL5 is secreted at low levels by three human GBM cell lines (FIG. 13A). Furthermore, we detected the high-level expression of CCL5 receptors CCR1 and/or CCR5 on human NK cells, macrophages, CD4⁺ T cells, and CD8⁺ T cells (FIGS. 13B-13E). To overcome the short half-life of CCL5 and allow high levels of CCL5 to build up in the TME, we generated a soluble bispecific fusion protein using a knob-into-hole design (19). One arm was designed to express the FDA-approved anti-EGFR antibody cetuximab for targeting EGFR⁺ tumor cells. The other arm was designed to contain human (h) or mouse (m) CCL5, encoded by two corresponding DNA sequences separated by a DNA sequence encoding a T2A self-cleaving peptide. S354C/T366W/K409A mutations of human IgG1 were included to generate the “knob” on the cetuximab-expressing arm, and Y349C/T366S/L368A/Y407V/F405K mutations were included to generate the “hole” on the CCL5-expressing arm (Cmab-hCCL5 or Cmab-mCCL5; (FIG. 1A) (19).

To validate the construct, we expressed Cmab-hCCL5 in CHO cells using lentiviral transduction. We first evaluated the binding of Cmab-hCCL5 to tumor cells. Because EGFR expression is heterogenous on tumor cells, we used U251T2 GBM cells, which express wild type EGFR (wtEGFR), U87ΔEGFR GBM cells, which express EGFRvIII, and A2780 ovarian tumor cells, which are EGFR⁻. Flow cytometry analysis using an anti-human Fc antibody showed that Cmab-hCCL5 selectively bound to tumor cells expressing wtEGFR or EGFRvIII, whereas Cmab-hCCL5 binding to EGFR⁻ A2780 cells was indistinguishable from that of IgG1 isotype control (FIG. 1). Using an anti-CCL5 antibody, we detected CCL5 on EGFR⁺ cell surfaces (FIG. 1C), suggesting that CCL5 was successfully indirectly bond to EGFR via cetuximab.

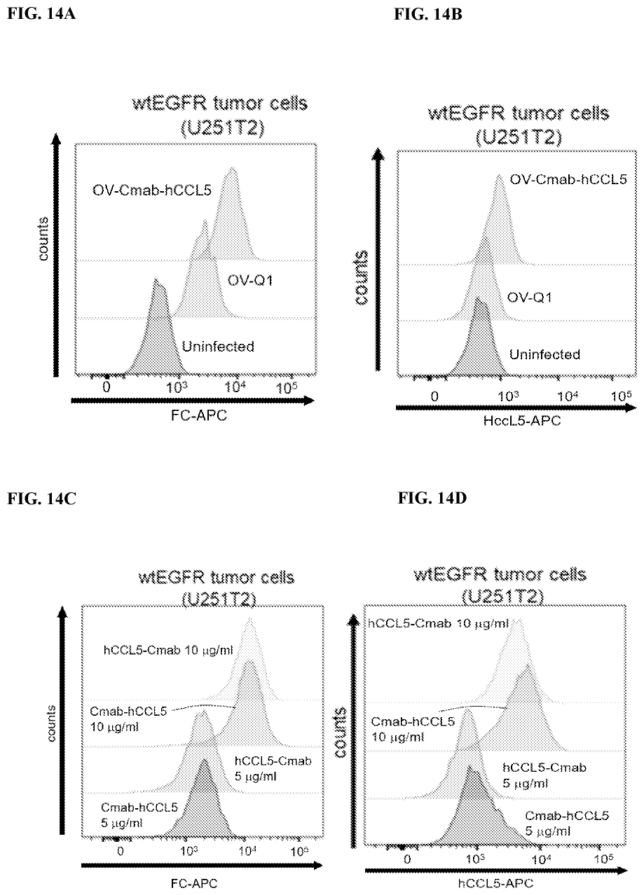

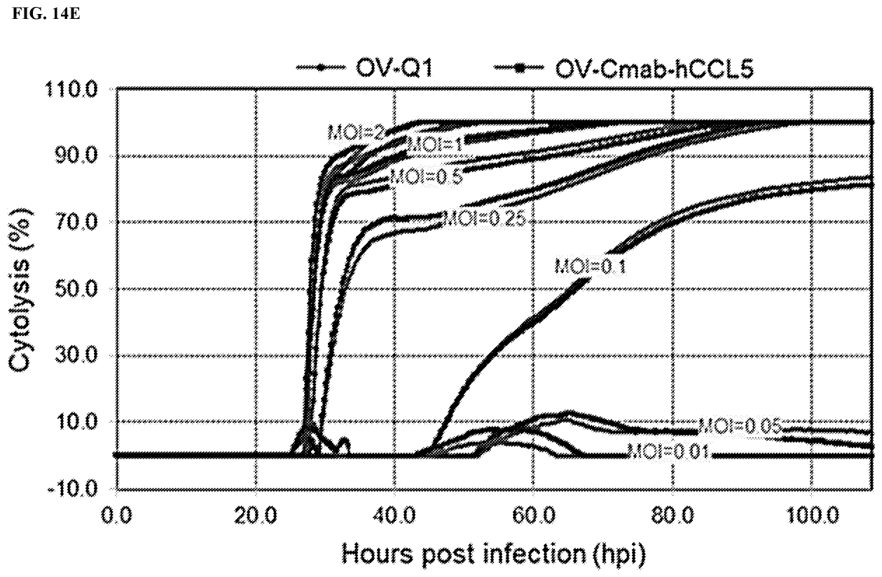

We then generated oHSV-expressing Cmab-hCCL5 using the parental OV-Q1 oHSV, which is double-attenuated with an inactivated ribonucleotide reductase gene (ICP6) and deletions of both copies of the neurovirulence gene (ICP34.5). These changes limit oHSV replication to tumor cells and reduce its neurovirulence (20). A DNA sequence encoding Cmab-CCL5 with a knob-into-hole IgG1 Fc was inserted into the ICP6 locus of OV-Q1, driven by the promoter of the HSV-1 immediate early gene IE4/5 to generate the OV-Cmab-CCL5 construct (FIG. 1D). The supernatants from OV-Cmab-hCCL5-infected and control (OV-Q1-infected) U251T2 GBM cells [48 hours post infection (hpi)] were collected for immunoblotting, along with supernatants from CHO cells with lentiviral expression of Cmab-hCCL5 or empty vector as positive and negative controls, respectively. hCCL5 and human IgG heavy chain both were detected in the supernatants of OV-Cmab-hCCL5-infected cells (FIG. 1E). Cmab-hCCL5 produced by OV-Cmab-hCCL5-infected U251T2 GBM cells was confirmed to bind to surface-expressed EGFR using anti-Fc and anti-CCL5 antibodies (FIG. 14A-14B). The concentrations of human CCL5 and Cmab from OV-Cmab-hCCL5-infected U251T2 GBM cells were quantified by ELISA, using CHO cell-derived fusion protein with known concentrations as standards. OV-Cmab-hCCL5-infected U251T2 GBM cells secreted CCL5 and Cmab as early as 12 hpi, with ˜32 nM human CCL5 and a similar concentration of human Cmab as maximum concentrations at 72 hpi (FIGS. 1F, 1G). We also generated the reverse version of OV-Cmab-CCL5 by switching the order of Cmab and CCL5 in the viral construct (OV-CCL5-Cmab). There was no difference between Cmab-CCL5 and CCL5-Cmab, either in the secretion of cetuximab or CCL5 (FIGS. 14C, 14D); OV-Cmab-hCCL5 was used in all subsequent experiments. Collectively, the data presented thus far demonstrate the successful generation of an OV-Cmab-hCCL5 construct that expresses a Cmab-hCCL5 bispecific fusion protein that can bind to EGFR on tumor cells.

To determine whether the addition of Cmab-hCCL5 to OV-Q1 affects its oncolytic capability, we infected U251T2 GBM cells with OV-Cmab-hCCL5 or OV-Q1 at a multiplicity of infection from 0.01 to 2. Real-time cell analysis indicated that the two viruses have a similar ability to lyse tumor cells (FIG. 14E).

### Example 3: Cmab-hCCL5 Promotes Migration of Human Immune Cells In Vitro

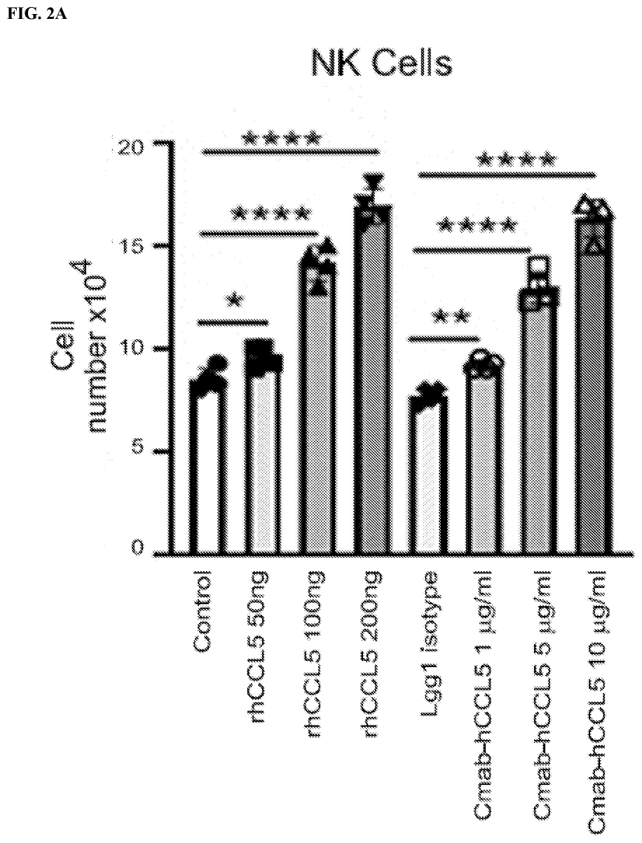

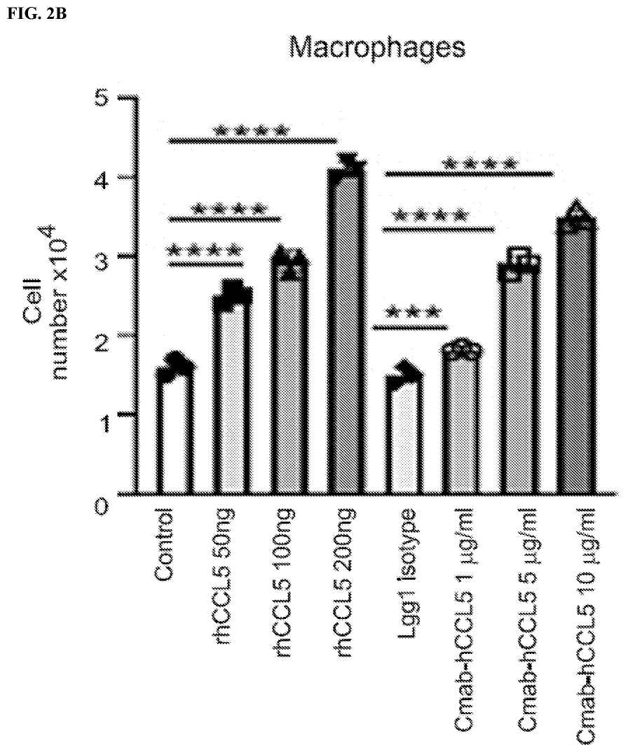

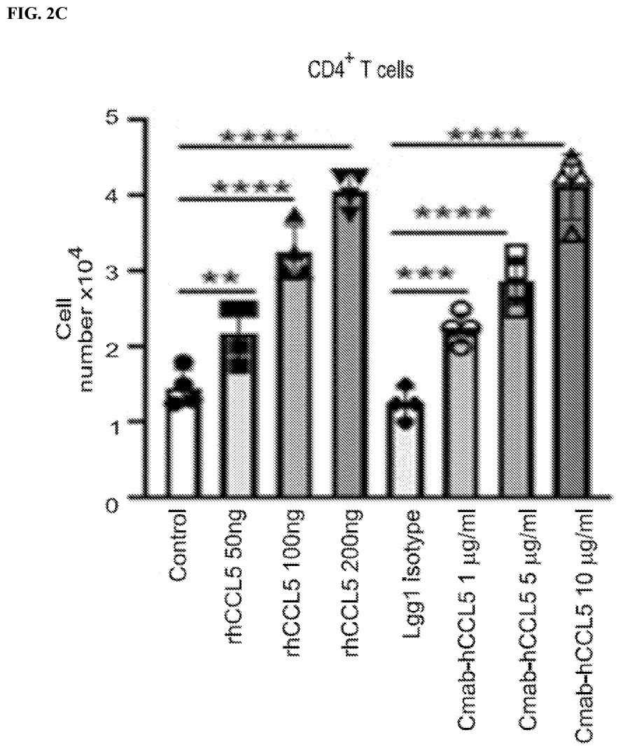

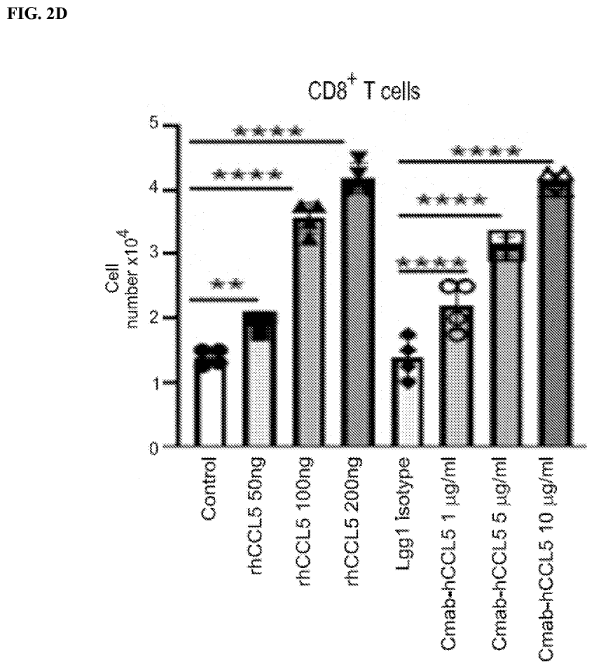

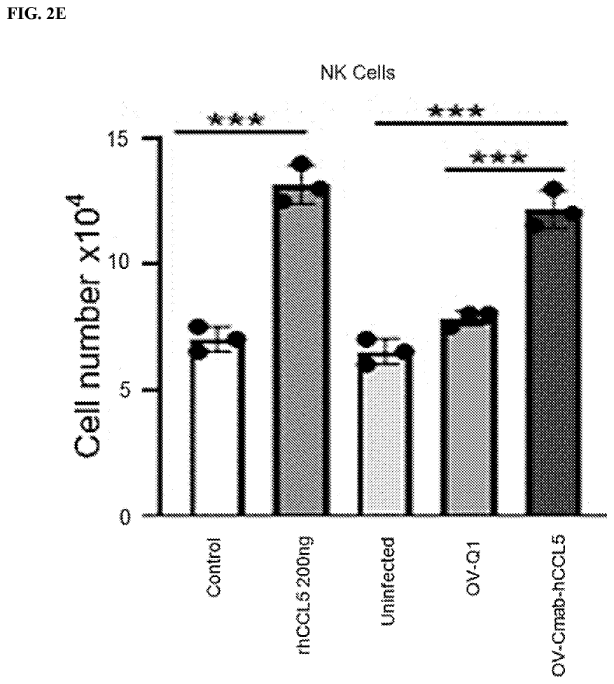

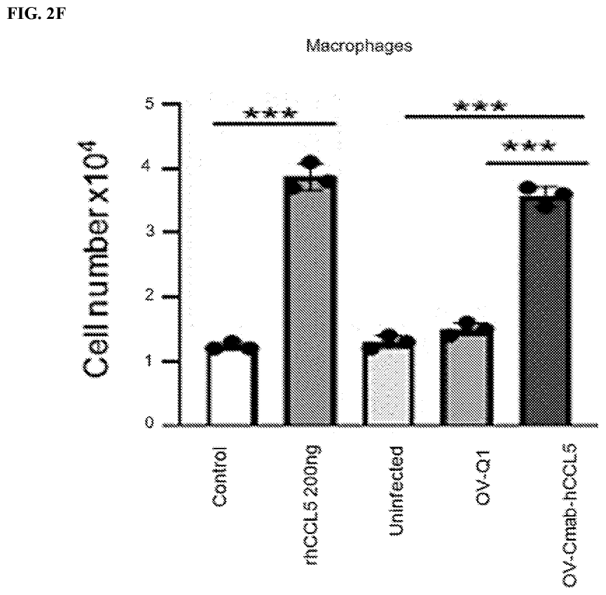

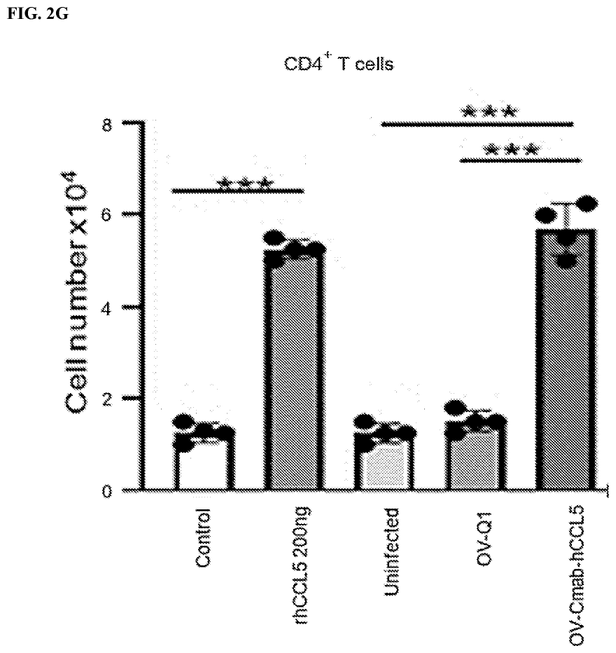

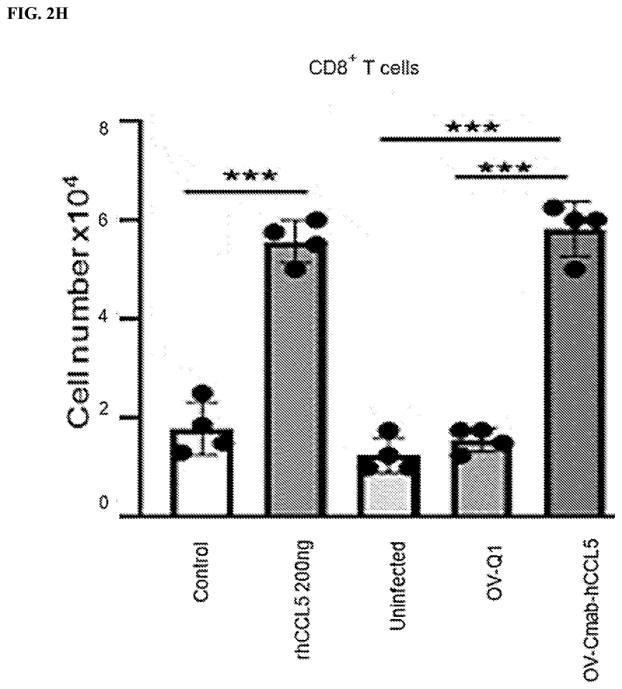

Next, we determined whether purified Cmab-hCCL5 could promote migration of immune cells in vitro using a transwell assay with recombinant human CCL5 as a positive control. Cmab-hCCL5 purified from supernatants of lentivirus-transduced CHO cells induced a dose-dependent increase in the migration of human NK cells, macrophages, CD4⁺ T cells, and CD8⁺ T cells when compared to an IgG1 isotype control (FIGS. 2A-2D). We also measured the ability of CmAb-hCCL5 produced by OV-CmAb-hCCL5-infected U251T2 GBM cells to regulate immune cell migration similarly in a transwell assay. At 48 hpi, the supernatants from OV-Cmab-hCCL5-infected U251T2 GBM cells, without removal of viruses, significantly increased the migration of human NK cells, macrophages, CD4⁺ T cells, and CD8⁺ T cells when compared to the supernatants from OV-Q1-infected U251T2 GBM cells (FIGS. 2E-2H). Our data demonstrate that Cmab-hCCL5 from OV-Cmab-hCCL5-infected cells can promote migration of human NK cells, macrophages, and T cells in vitro and independent of cell contact.

### Example 4: Cmab-hCCL5 Induces Human NK Cell-Mediated ADCC Against EGFR⁺ Tumor Cells

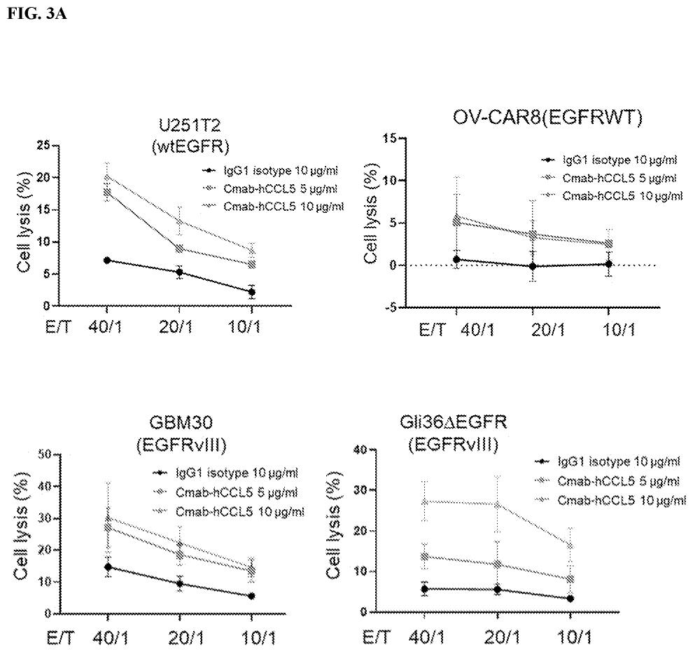

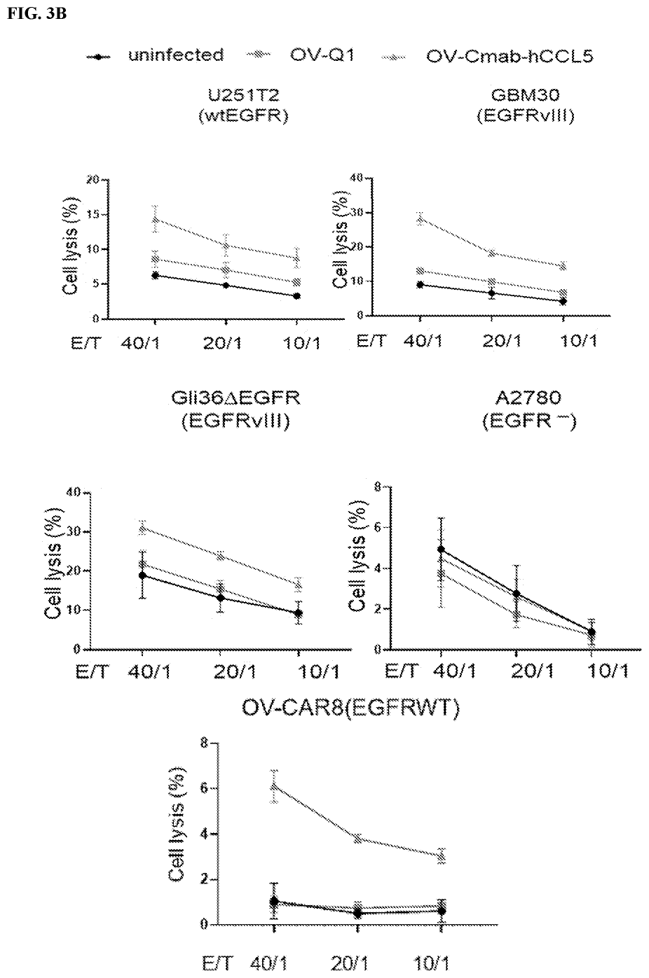

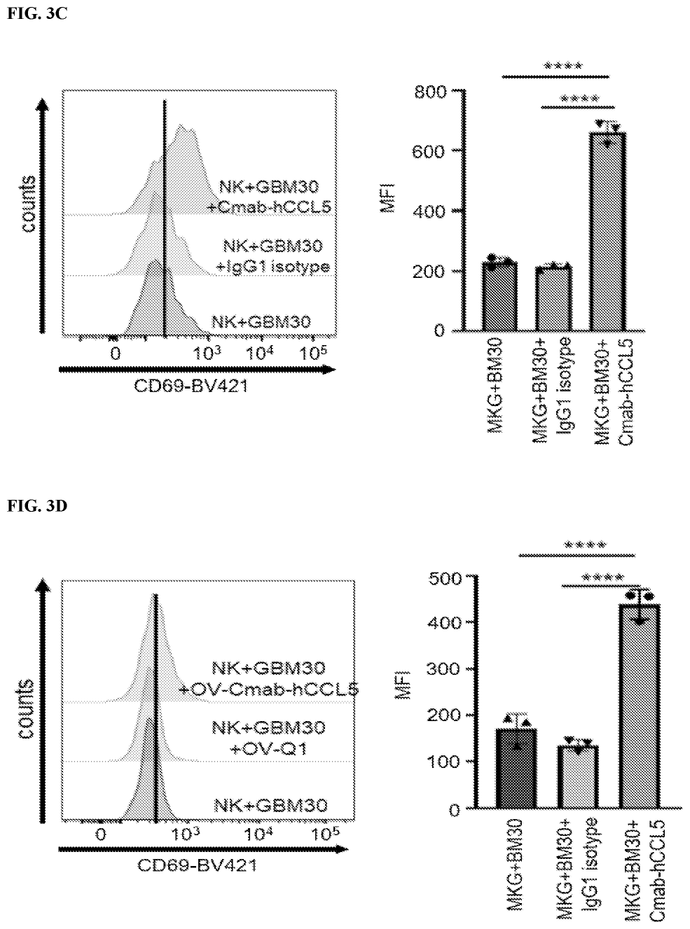

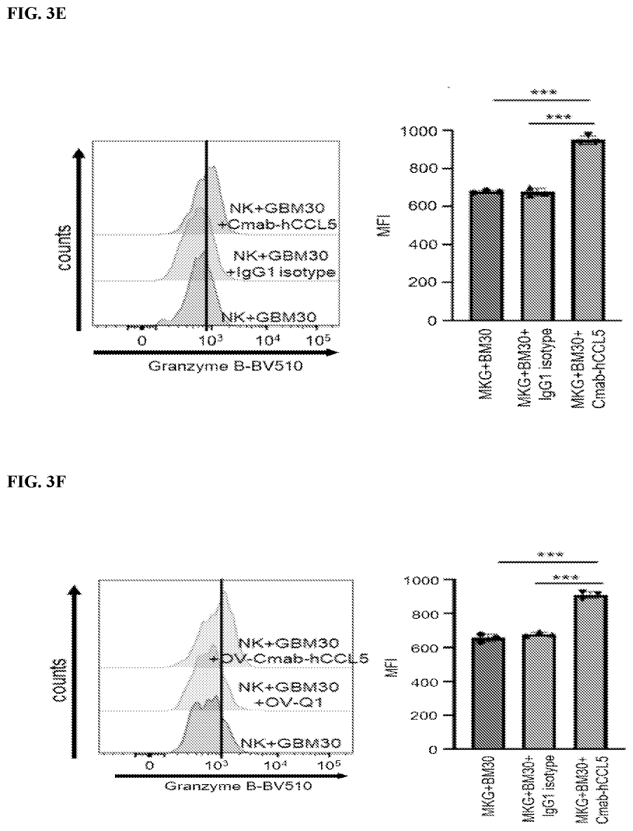

NK cells have an antitumor function because they possess natural cytotoxicity and can carry out ADCC in the presence of some antibodies. To investigate the role of Cmab-hCCL5 in regulating NK cell ADCC, we conducted a standard ⁵¹Cr release assay by co-culturing human NK cells with U251T2 (wtEGFR), GBM30, or Gli36ΔEGFR (EGFRvIII) GBM cells labeled with ⁵¹Cr in the presence or absence of Cmab-hCCL5 purified from lentivirus-transduced CHO cells for 30 min. Cmab-hCCL5 significantly induced NK cell ADCC targeting both wtEGFR and EGFRvIII tumor cells when compared to an IgG1 isotype control (FIG. 3A). Similarly, the supernatants from OV-Cmab-hCCL5-infected U251T2 GBM cells, without removal of viruses, induced significantly more cytolysis compared to the supernatants from OV-Q1-infected cells targeting wtEGFR and EGFRvIII GBM cells, while lacking such an effect when targeting EGFR⁻ cells (FIG. 3B). To validate these data, we co-cultured GBM30 cells with human NK cells treated with Cmab-hCCL5 purified from lentivirus-infected CHO cells for 6 hours, and flow cytometry was used to measure the expression of CD69 and granzyme B, two NK cell activation markers. Cmab-hCCL5 significantly increased the expression of CD69 on NK cells compared to an IgG1 isotype control or GBM30 cells alone (FIG. 3C). Also, the fraction of CD69⁺ NK cells was higher after culturing with supernatants collected from the OV-Cmab-hCCL5-infected U251T2 GBM cells, compared to culture with supernatants collected from uninfected or OV-Q1-infected U251T2 GBM cells. (FIG. 3D). Using the same experimental design, we also observed increased granzyme B expression after exposure to Cmab-hCCL5 purified from lentivirus-transduced CHO cells (FIG. 3E) or the supernatant from OV-Cmab-hCCL5 infected U251T2 GBM cells relative to controls (FIG. 3F).

### Example 5: Cmab-hCCL5 Enhances Human Macrophage ADCP Against GBM Cells

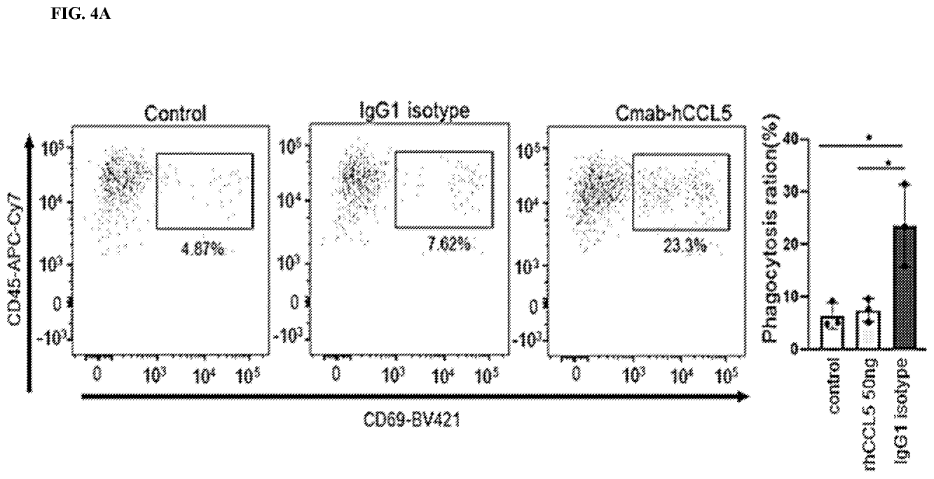

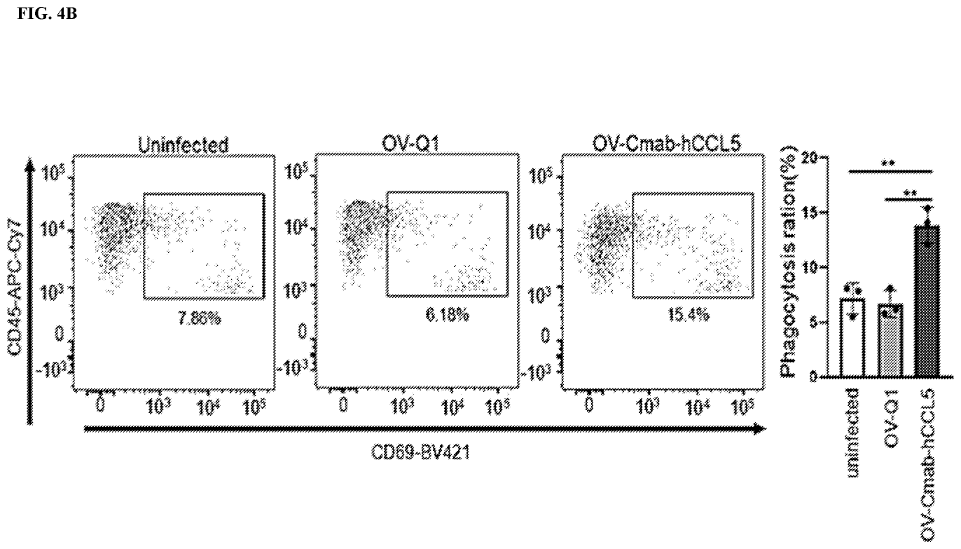

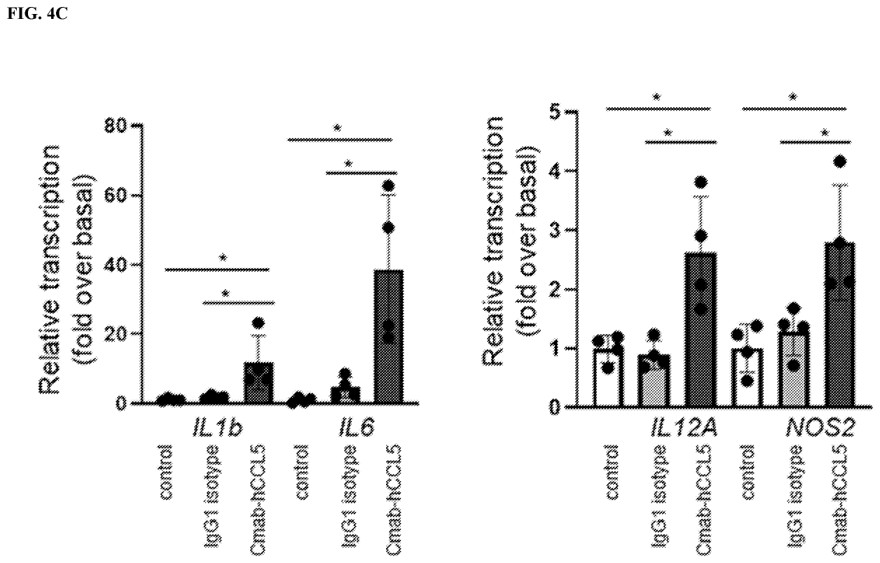

Macrophages play an important role in the TME by mediating ADCP and releasing cytokines. We, therefore, determined the effect of Cmab-hCCL5 on macrophages. GBM30 cells were co-cultured with human M-CSF-treated primary monocyte-derived macrophages for 4 hours with or without Cmab-hCCL5 purified from supernatants of lentivirus-infected CHO cells. Flow cytometry revealed that Cmab-hCCL5 induced a significantly higher level of ADCP against GBM30 cells compared to an IgG1 isotype control or the vehicle control (FIG. 4A). In addition, the ADCP assay was repeated with the non-concentrated and virus-containing supernatants from OV-Cmab-hCCL5- or OV-Q1-infected U251T2 GBM cells. Consistent with the effects of purified Cmab-hCCL5, the supernatants from OV-Cmab-hCCL5-infected U251T2 GBM cells dramatically induced ADCP compared to supernatants from OV-Q1-infected or uninfected U251T2 GBM cells (FIG. 4B). We also measured cytokine expression levels that can indicate the activation status of macrophages. Compared to an IgG1 isotype control, Cmab-hCCL5 treatment significantly increased IL-1B, IL-6, IL-12, and NOS2 expression in human macrophages, as measured by real-time RT-PCR (FIG. 4C).

### Example 6: OV-Cmab-hCCL5 Improves Oncolytic Virotherapy in a Xenograft GBM Animal Model

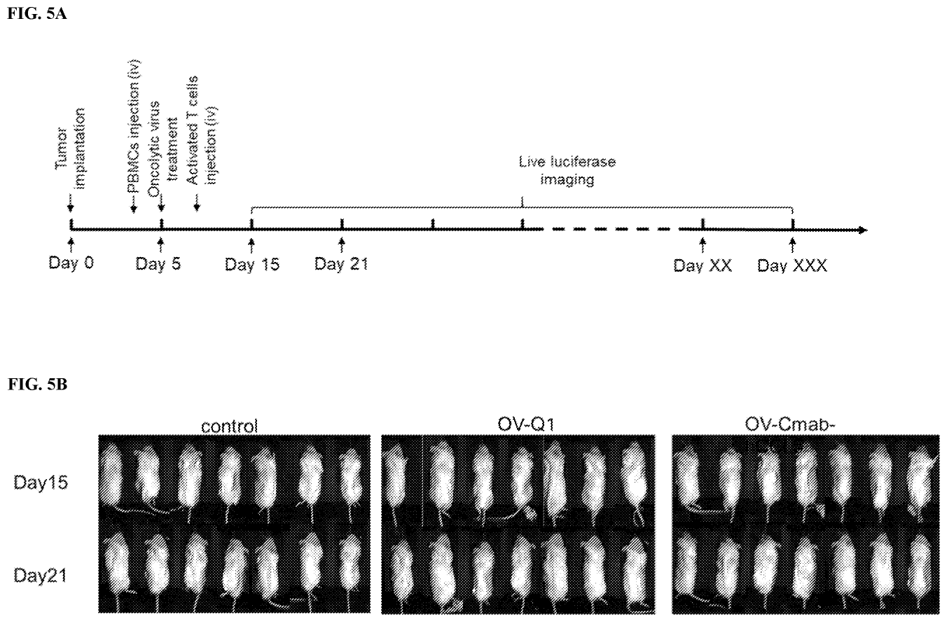

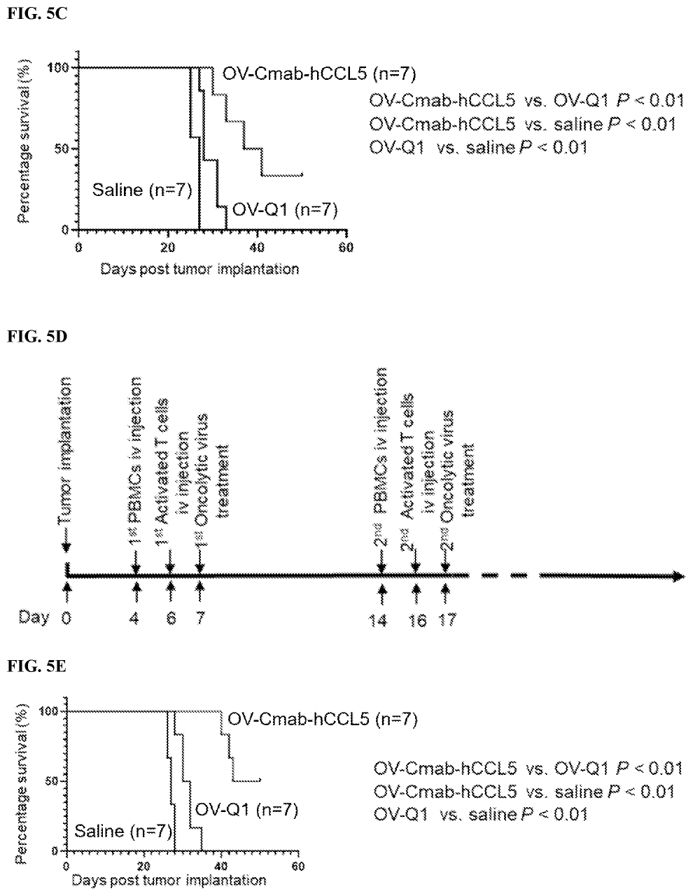

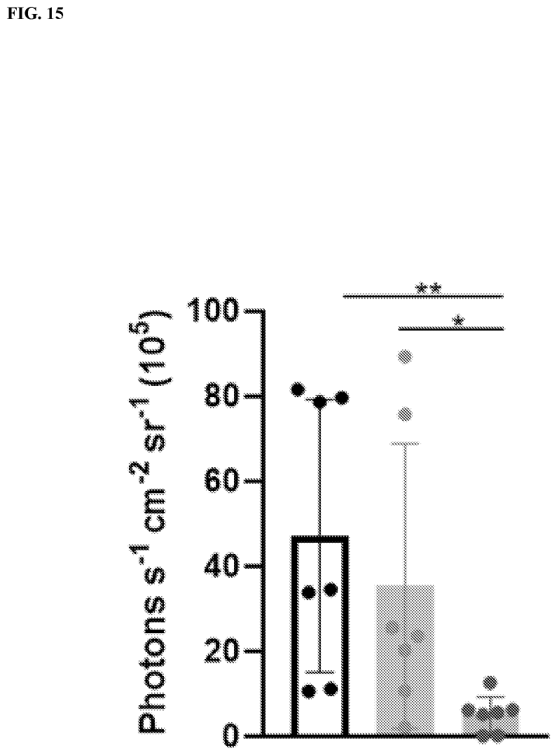

To evaluate the efficacy of OV-Cmab-hCCL5 for the in vivo treatment of GBM, we used a previously described orthotopic model of human GBM created by intracranial injection of 1×10⁵ fly luciferase (FFL) gene-expressing human GBM30-FFL cells into NOD-scid IL2Rγ^{null} (NSG) mice(21). To mimic the human immune system, 4 days after tumor implantation, we delivered 1×10⁶ peripheral blood mononuclear cells (PBMCs) isolated from a single donor to each mouse by intravenous injection. On Day 6, 1×10⁶ activated T cells derived from the same donor were delivered to each mouse by intravenous injection. Seven days after tumor implantation, the mice received an intracranial injection with OV-Cmab-hCCL5 or OV-Q1 at 2×10⁵ plaque-forming units (pfu) per mouse, or saline as a placebo control. Tumor progression was monitored by luciferase-based imaging 15 and 21 days after tumor implantation (FIG. 5A). This one-cycle treatment with OV-Cmab-hCCL5 was significantly more effective than OV-Q1 or vehicle control at inhibiting the progression of GBM tumors in vivo (FIG. 5B and FIG. 15). OV-Q1 moderately slowed GBM progression compared to vehicle, while OV-Cmab-hCCL5 significantly prolonged median survival compared to both saline control and OV-Q1 treatment (FIG. 5C). We then performed a similar experiment with a second treatment cycle on Day 14 to Day 17 as indicated (FIG. 5D) and evaluated the therapeutic efficacy. This two-cycle treatment with OV-Cmab-hCCL5 also significantly improved the survival of GBM mice compared to the two-cycle OV-Q1 or saline treatment (FIG. 5E). Of note, the two-cycle OV-Cmab-hCCL5 treatment provided more protection to GBM-bearing mice compared to the one-cycle treatment, as the median survival day was prolonged from 39 days to 47 days (FIG. 5E vs. FIG. 5C). Collectively, the data demonstrate that OV-Cmab-hCCL5 improves the efficacy of oncolytic virotherapy in vivo, using a mouse GBM xenograft model.

### Example 7: Cmab-mCCL5 Promotes Murine Immune Cell Migration and Activation; OV-Cmab-mCCL5 Prolongs Survival of Glioblastoma-Bearing Mice in an Immunocompetent Mouse Model

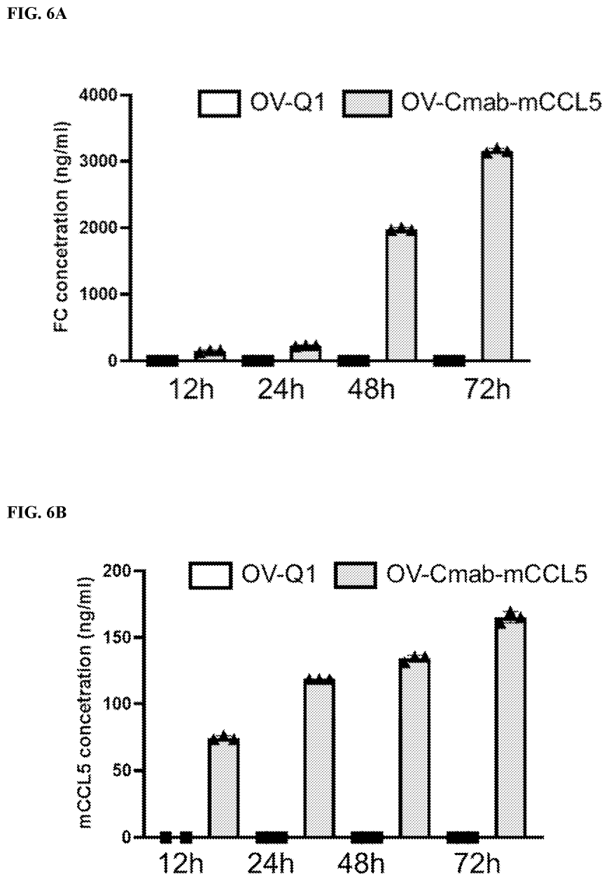

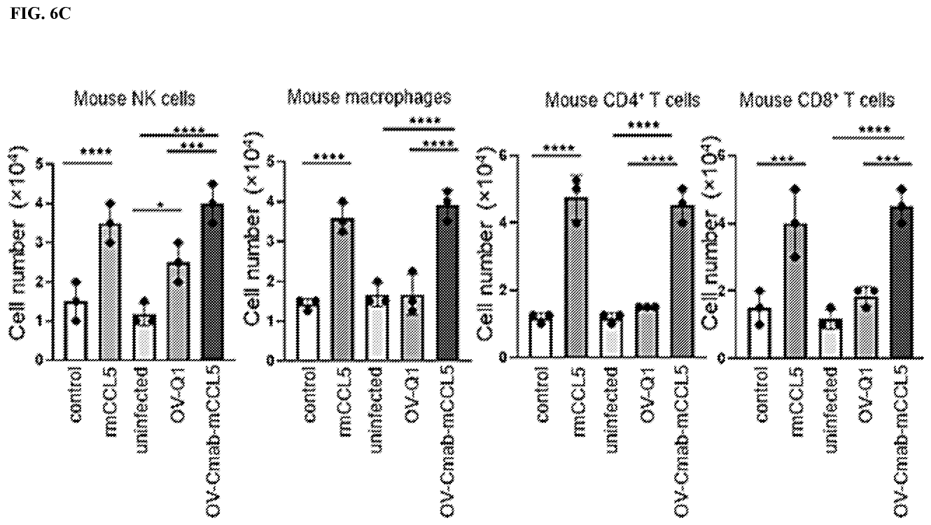

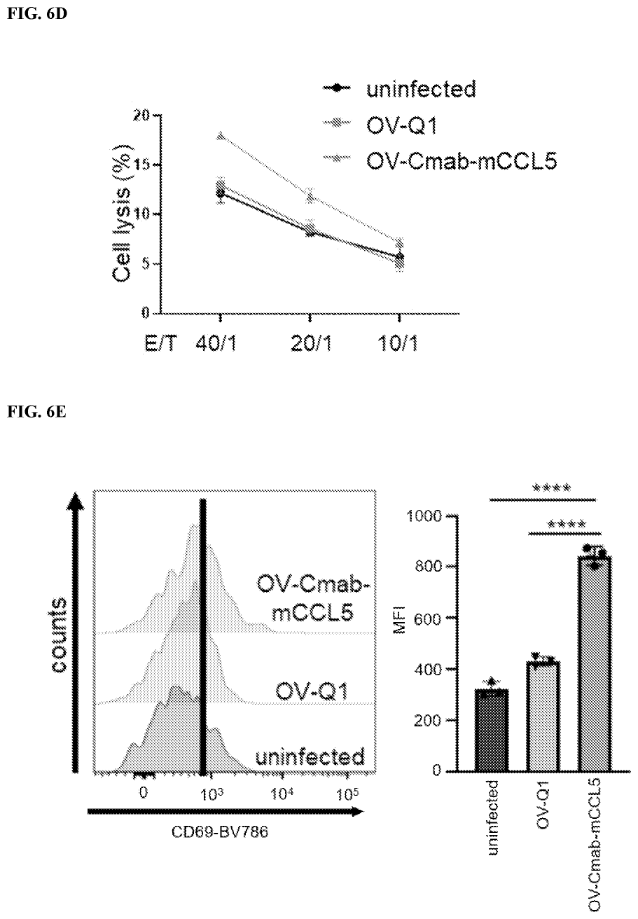

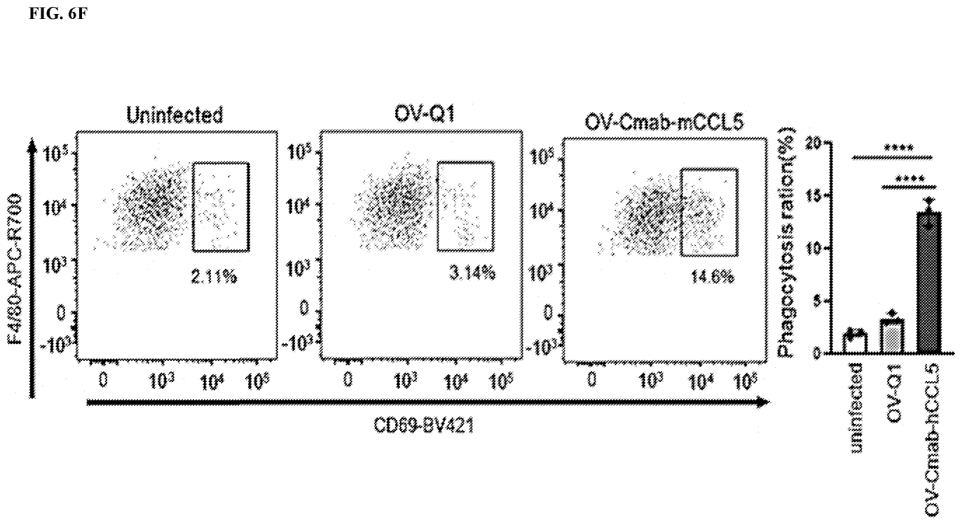

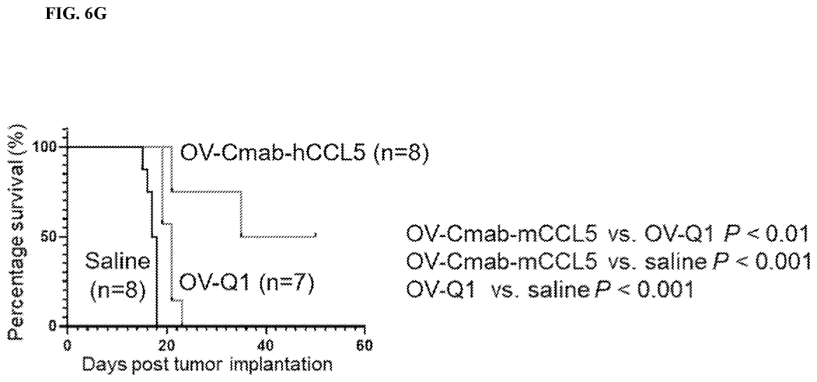

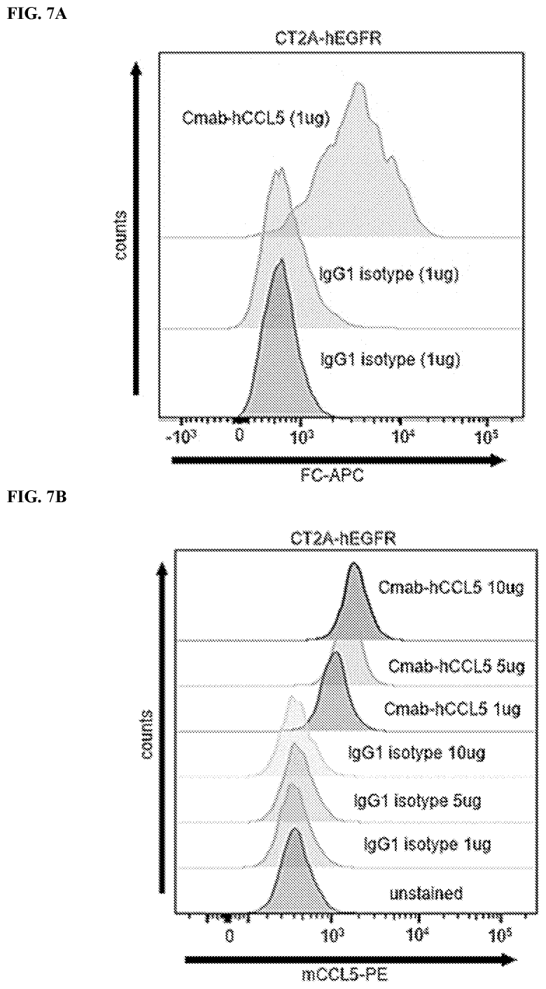

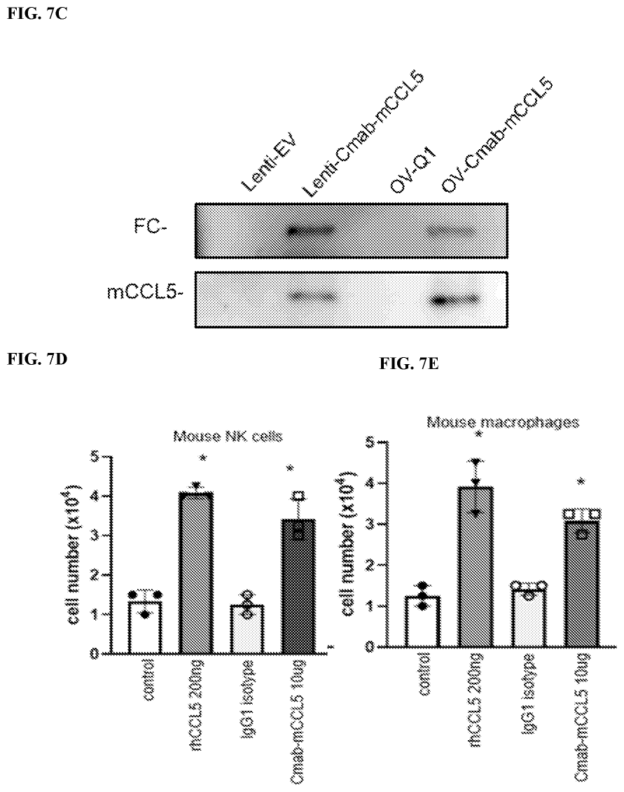

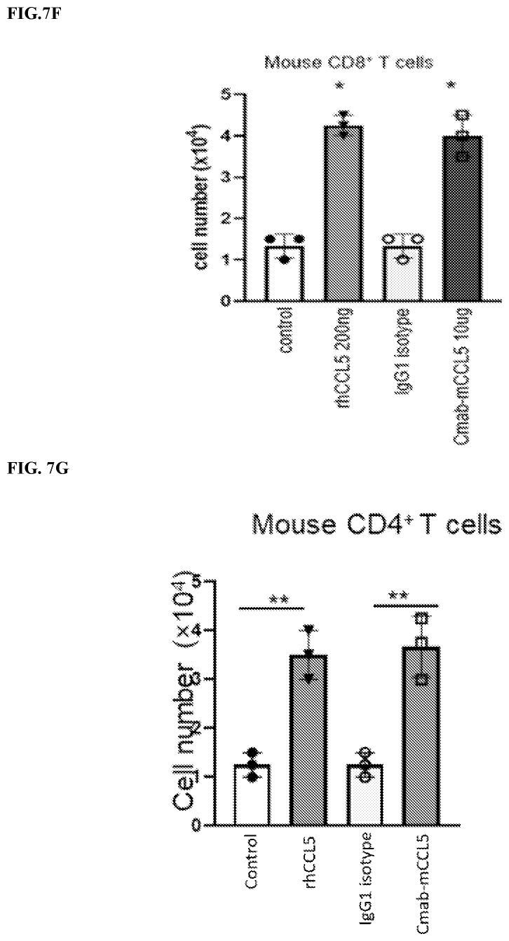

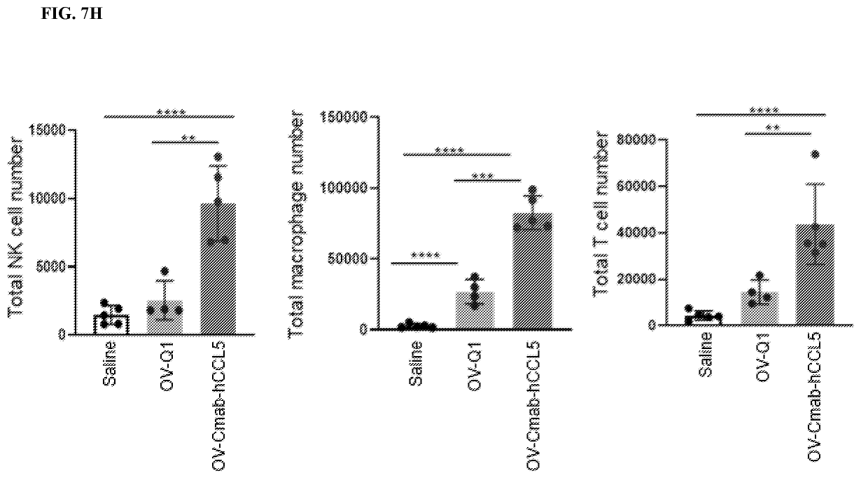

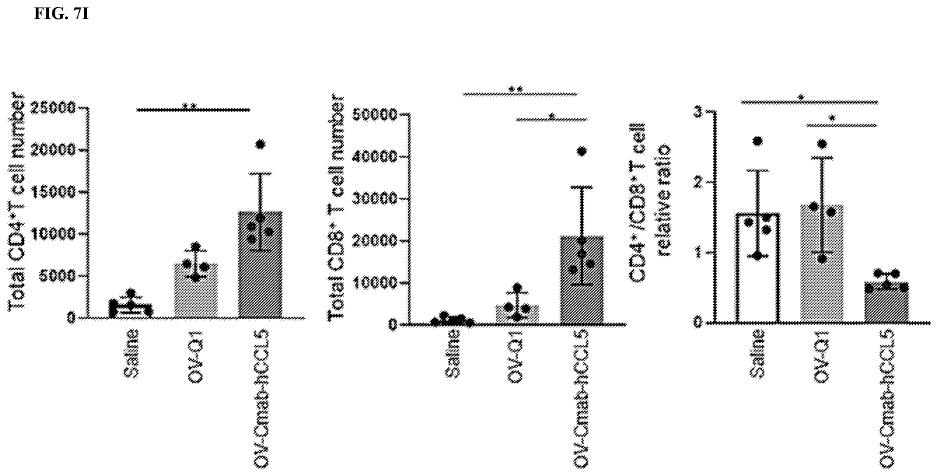

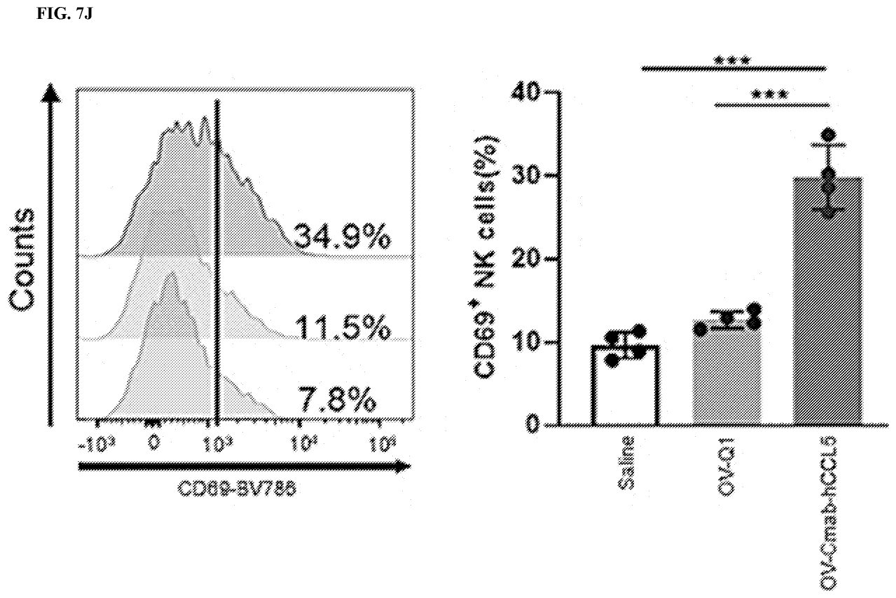

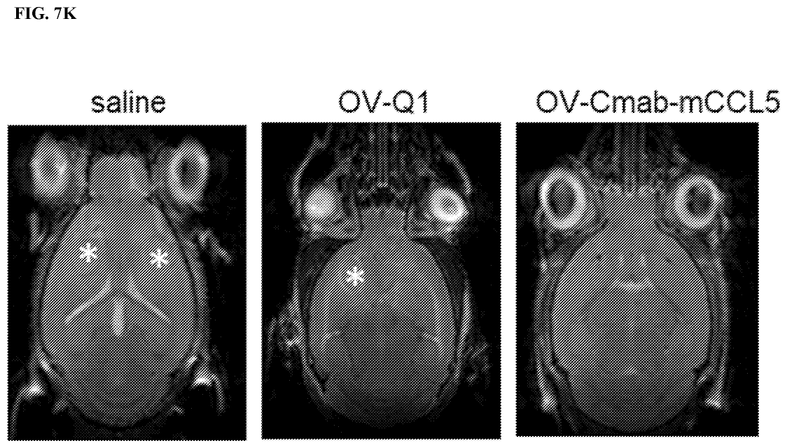

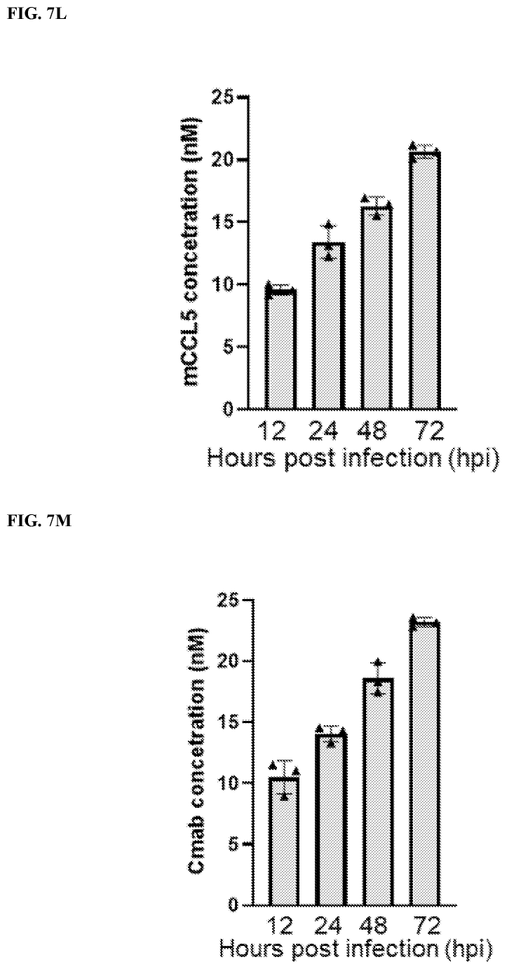

To test our oncolytic virotherapy in a fully immunocompetent mouse model of GBM, we replaced human CCL5 in OV-Cmab-hCCL5 with murine CCL5 to generate OV-Cmab-mCCL5. We also expressed human EGFR in the murine GBM CT2A cell line (CT2A-hEGFR) so that cetuximab could bind to murine GBM cells. Indeed, Cmab-mCCL5 purified from lentivirus-infected CHO cells could bind to CT2A-hEGFR cells (FIGS. 7A, 7B). We also confirmed that Cmab-mCCL5 in supernatants derived from OV-Cmab-mCCL5-infected CT2A-hEGFR cells was detectable by immunoblotting (FIG. 7C). The OV-Cmab-mCCL5-infected CT2A-hEGFR cells released mCCL5 as early as 12 hours post-infection, and the yields reached levels over 9 nM (100 ng/ml) at 24 hours post infection, and the levels were even higher after 72 hours post infection at over 20 nM (160 ng/ml) (FIG. 7L, 7M). Cetuximab was also produced (FIG. 7D). Similar to our results for Cmab-hCCL5, Cmab-mCCL5 purified from lentivirus-transduced CHO cells and that produced by OV-Cmab-mCCL5-infected CT2A-hEGFR cells both increased the migration of murine NK cells, macrophages, and T cells (FIGS. 7D, 7G and 6C).

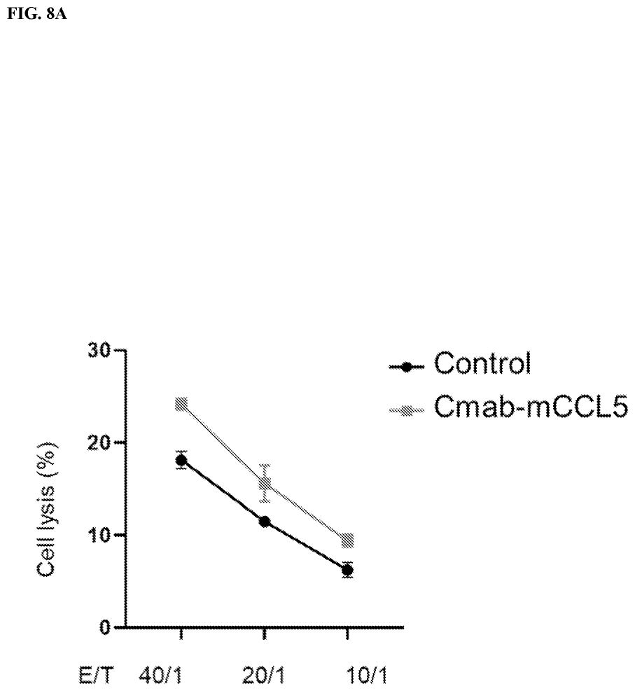

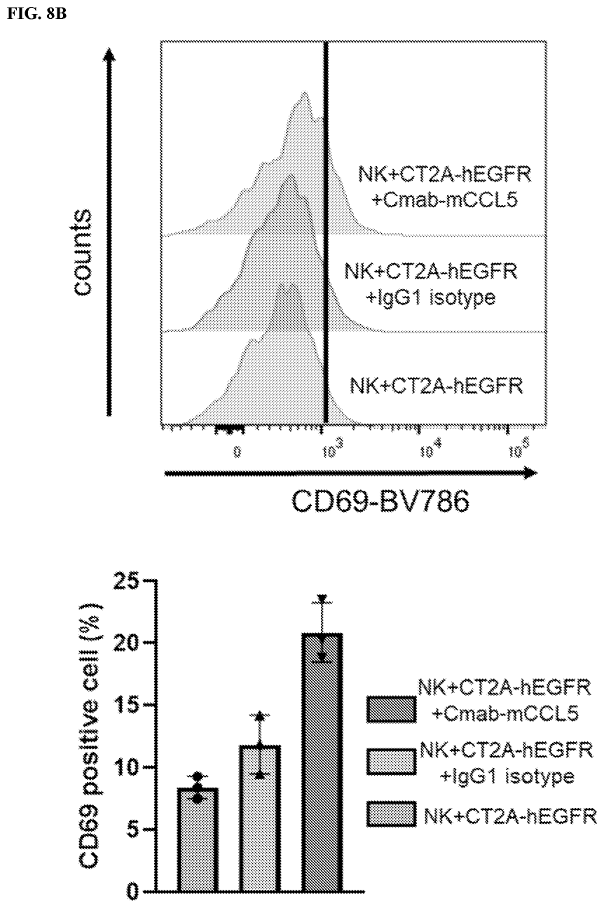

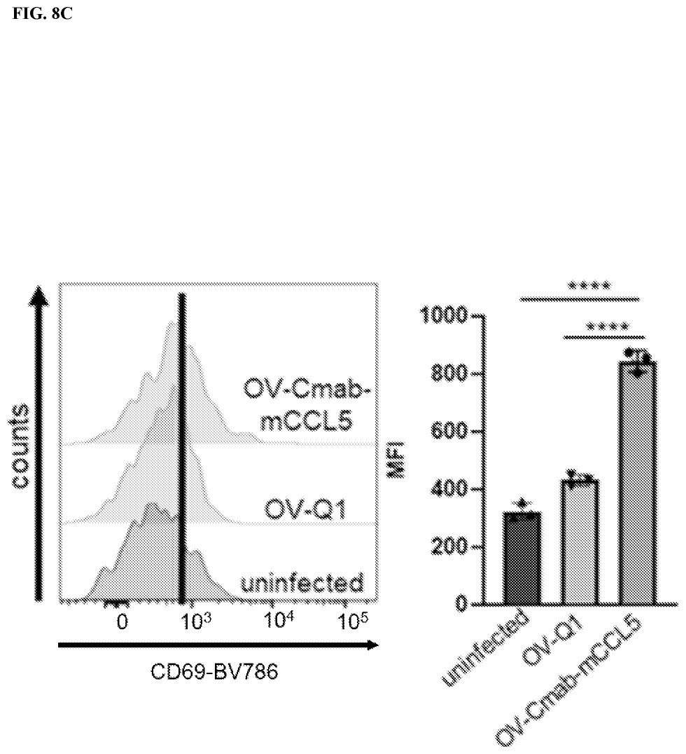

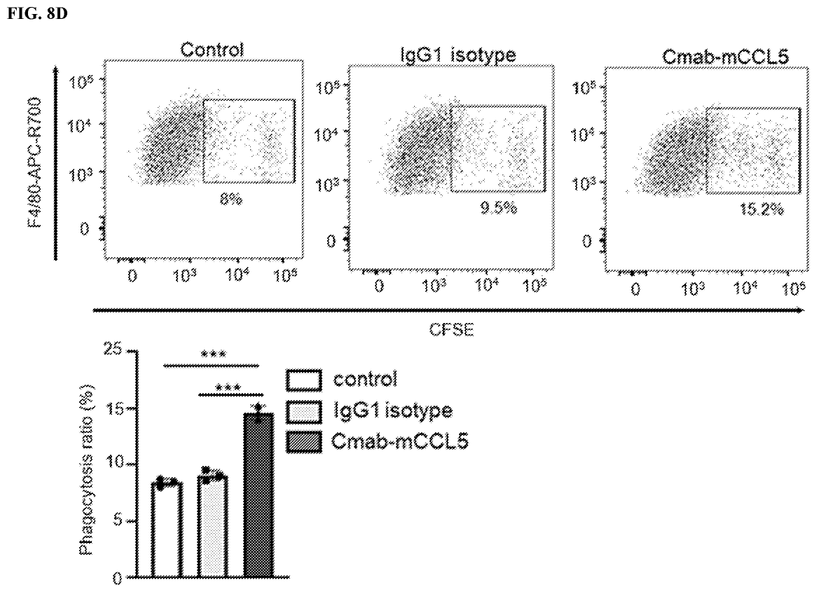

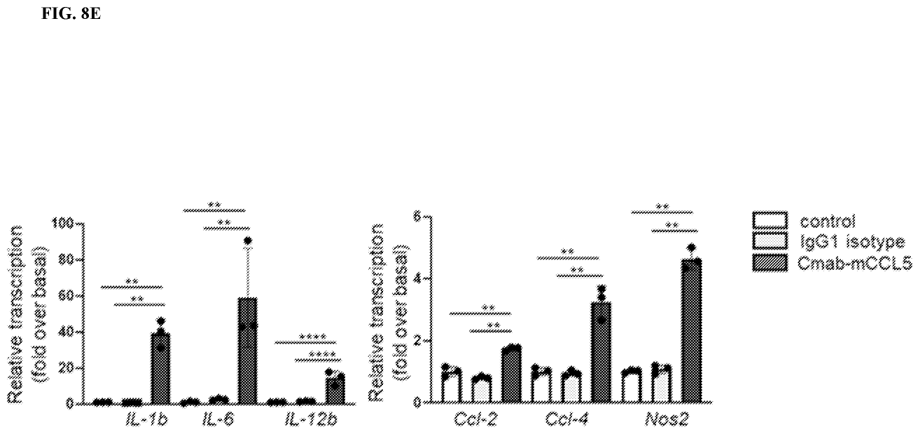

Cmab-mCCL5 purified from lentivirus-transduced CHO cells and Cmab-mCCL5- and virus-containing supernatants from OV-Cmab-mCCL5-infected CT2A-hEGFR cells significantly enhanced the ADCC function of NK cells targeting CT2A-hEGFR cells, compared to corresponding controls (FIGS. 8A and 6D). These data are consistent with the observed increase in CD69 expression on murine NK cells (FIG. 8C, 6E), as shown above for human NK cells following exposure to Cmab-hCCL5 (FIGS. 3A-3F).

Cmab-mCCL5 purified from lentivirus-transduced CHO cells and Cmab-mCCL5- and virus-containing supernatants from OV-Cmab-mCCL5-infected CT2A-hEGFR cells also significantly increased the ADCP of murine macrophages targeting CT2A-hEGFR cells, compared to corresponding controls (FIGS. 8D, 6F). Furthermore, purified Cmab-mCCL5 significantly upregulated expression of IL-1b, IL-6, IL-12b, Ccl-2, Ccl-4, and Nos2 in murine macrophages co-cultured with Cmab-mCCL5-treated CT2A-hEGFR cells (FIG. 8E). These data are also similar to those obtained for human macrophages following exposure to Cmab-hCCL5 (FIGS. 4A-4C).

The murine system that we established consisting of OV-Cmab-mCCL5 and CT2A-hEGFR cells allowed us to evaluate the efficacy of OV-Cmab-mCCL5 in an immunocompetent model, in which CT2A-hEGFR cells were injected i.e. into wild-type C57BL/6J mice. Treatment with OV-Cmab-mCCL5 significantly prolonged median survival compared to OV-Q1 or vehicle control. The median survival was extended from 17.5 days to 42.5 days (FIG. 6G).

### Example 8: OV-Cmab-mCCL5 Induces Infiltration of Innate and Adaptive Murine Immune Cells into GBM TME that Contributes to Tumor Eradication

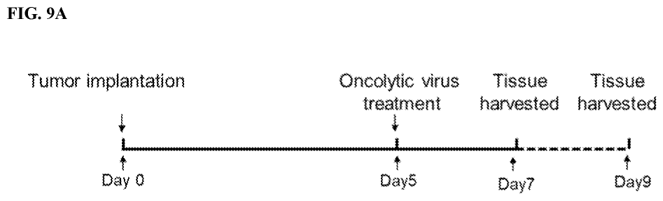

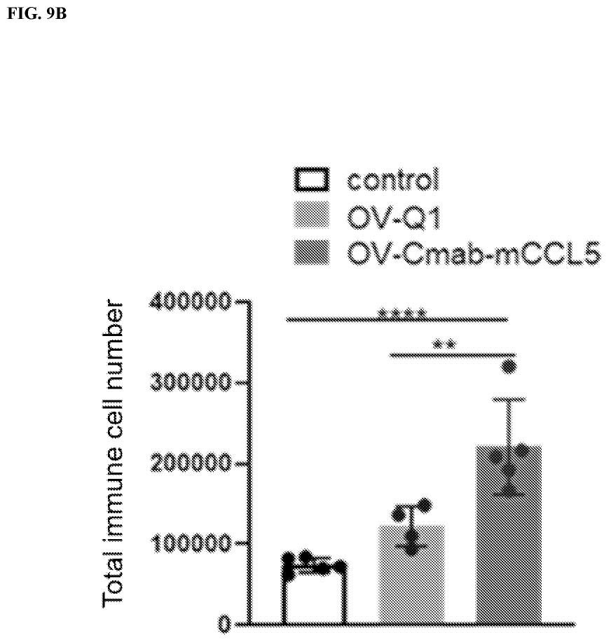

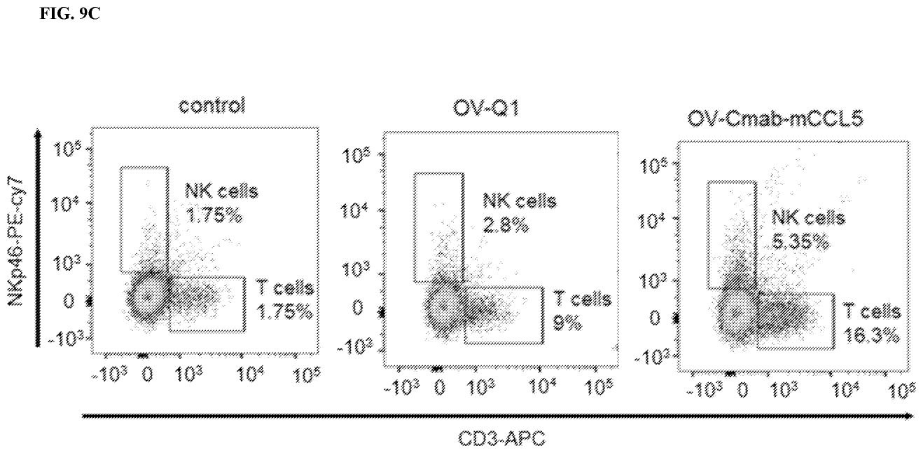

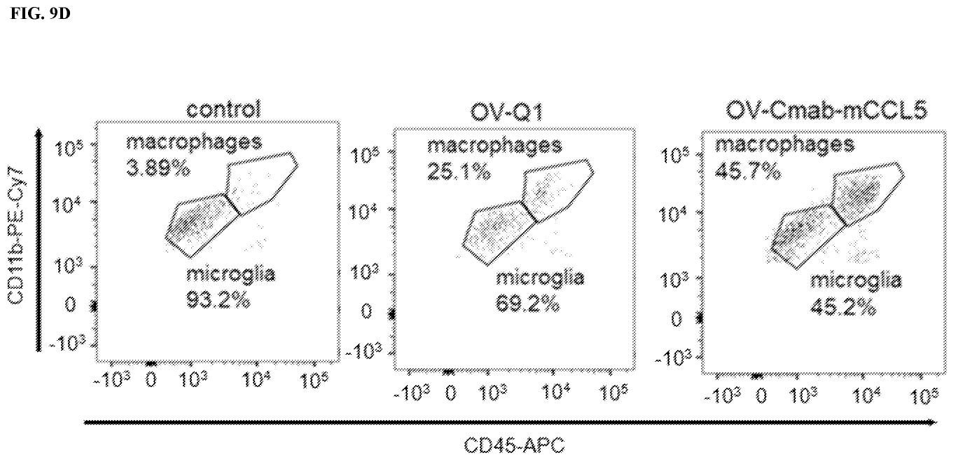

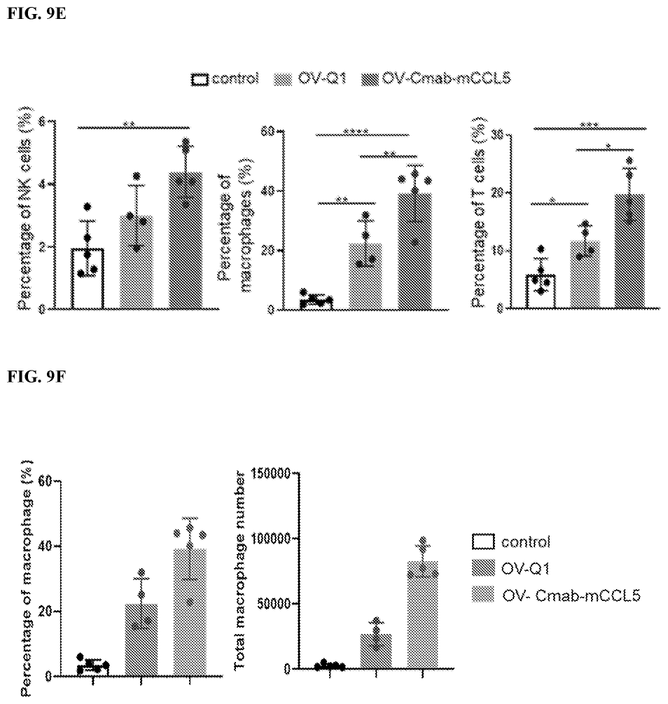

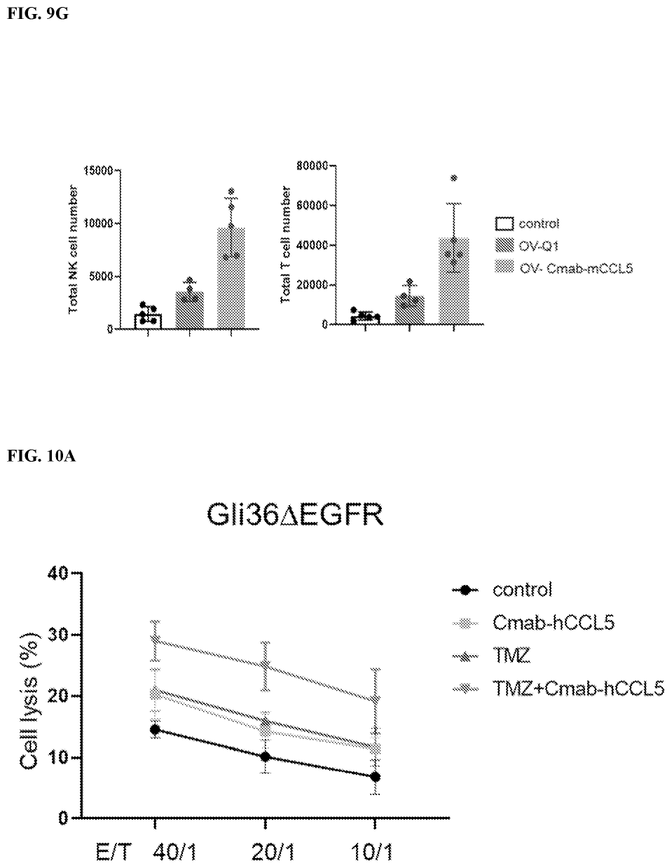

To determine whether OV-Cmab-mCCL5 enhances innate and adaptive immunocyte infiltration in vivo, we utilized our CT2A-hEGFR immunocompetent GBM mouse model. Five days after tumor cell implantation, the mice received an i.e. injection with OV-Cmab-mCCL5 or OV-Q1 at 2×10⁵ pfu per mouse, or saline vehicle control, followed by euthanasia on Day 7. The total number of immune cells, NK cells, macrophages, and T cells isolated from the brain were measured by flow cytometry. The total number of immune cells significantly increased in OV-Cmab-mCCL5-treated mice compared to either the vehicle control mice or OV-Q1-treated mice (FIG. 9B). OV-Cmab-mCCL5 treatment recruited more NK cells, macrophages, and total T cells to the brain than either the OV-Q1 or vehicle control treatment (FIG. 7H and FIGS. 9C-9E). Furthermore, the number of CD4⁺ T cells and CD8⁺ T cells were increased while the CD4⁺/CD8⁺ T cell ratio was decreased in the OV-Cmab-mCCL5 treatment group compared to either the OV-Q1 or vehicle control treatment groups (FIG. 7I). These data indicated that OV-Cmab-mCCL5 promotes innate and adaptive immune cell infiltration into the GBM TME. We also measured in vivo NK cell activation via assessing CD69 expression by flow cytometry. Our results showed that OV-Cmab-mCCL5 treatment significantly increased the percentage of CD69⁺ NK cells compared to OV-Q1 or saline treatment, indicating that OV-Cmab-mCCL5 activated NK cells in vivo (FIG. 7J). We also detected a positive effect of OV-Cmab-mCCL5 injected in only one hemisphere of the brain on the other hemisphere when both hemispheres were implanted with an equal number of GBM cells. Magnetic resonance imaging (MRI) showed that OV-Cmab-mCCL5 completely eradicated tumors on both sides of the brain, while OV-Q1 only completely eliminated the tumor on the side treated with OV-Q1 while the tumor on the untreated side decreased in size but did not disappear. Our data suggested that OV-Cmab-mCCL5 has a better abscopal effect of controlling tumor metastasis than OV-Q1 (FIG. 7K).

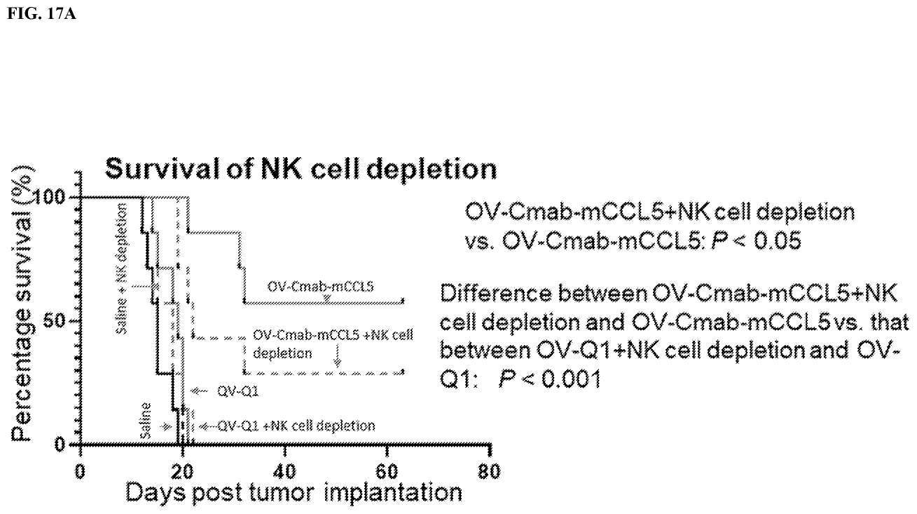

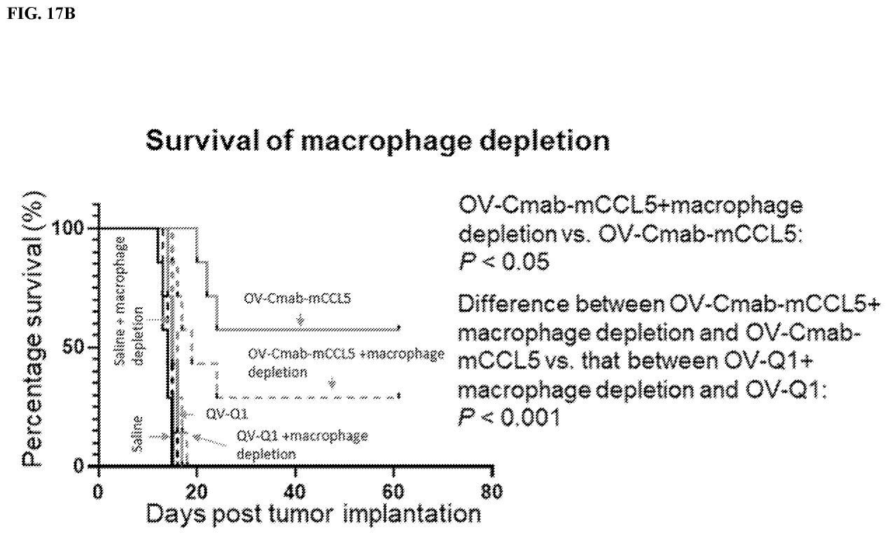

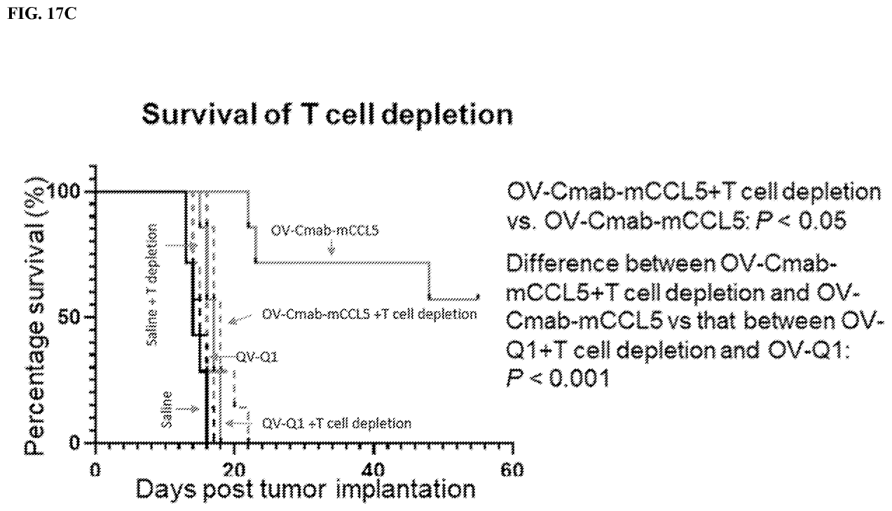

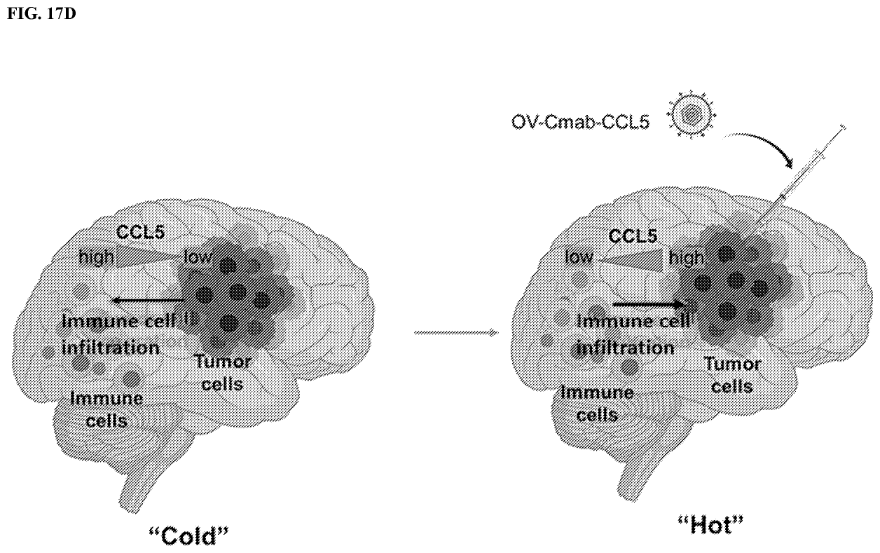

To determine the contribution of different immune cells toward the efficacy of the OV-Cmab-mCCL5 treatment, we repeated the survival studies in the immunocompetent GBM mouse model with or without depletion of NK cells, macrophages, or T cells. Depletion of NK cells or macrophages had no effect on the survival of either the OV-Q1 or vehicle control group, which may be partially explained by the aggressive nature of this GBM model, in which nearly all untreated mice died by approximately day 18 after tumor implantation. In the OV-Cmab-mCCL5 mice, the depletion of NK cells or macrophages resulted in a decrease in the percentage of OV-Cmab-mCCL5-treated mice surviving, with the mice dying earlier compared to the group without depletion of these innate immune cells (FIG. 17A, 17B). The median survival in NK or macrophage depletion group was also shortened, and the effect of OV-Cmab-mCCL5 was significantly impaired by the depletions (FIG. 17A, 17B). These data are also consistent with our in vitro data showing that Cmab-mCCL5 produced by OV-Cmab-mCCL5- or OV-Cmab-hCCL5-infected GBM cells significantly induced NK cell migration and ADCC and macrophage migration and ADCP (FIG. 6C, 6D, 6F). Depletion of T cells completely abolished the protective effect of OV-Cmab-mCCL5, indicating that adaptive immune cell activation is also important for the protective effects of OV-Cmab-mCCL5 treatment (FIG. 17C). Collectively, our data show that OV-Cmab-mCCL5 induces infiltration of both innate and adaptive cytolytic immune cells into the GBM TME, both of which contribute to the protective effect of OV-Cmab-mCCL5. This immune infiltration induction may be due to high CCL5 expression from OV-Cmab-mCCL5-infected GBM cells, which results in gradient change of CCL5 with a high level in tumor while a low level in the other parts of TME (FIG. 17D). Notably, the T cells of the adaptive immune system seemed to be more important than the NK cells and macrophages of the innate immune system for the antitumor effects of OV-Cmab-mCCL5.

### Example 9: The Combination of OV-Cmab-CCL5 with TMZ Demonstrates a Synergetic Effect In Vitro and In Vivo

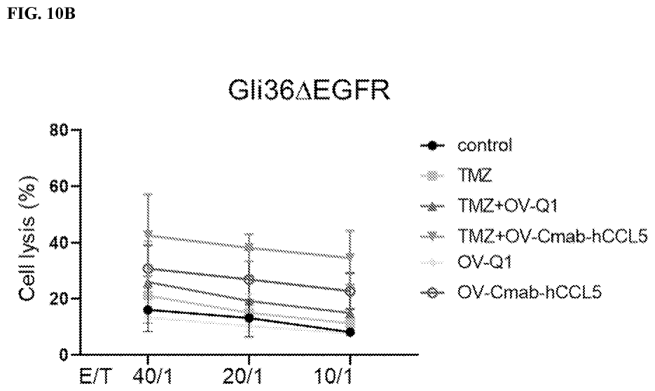

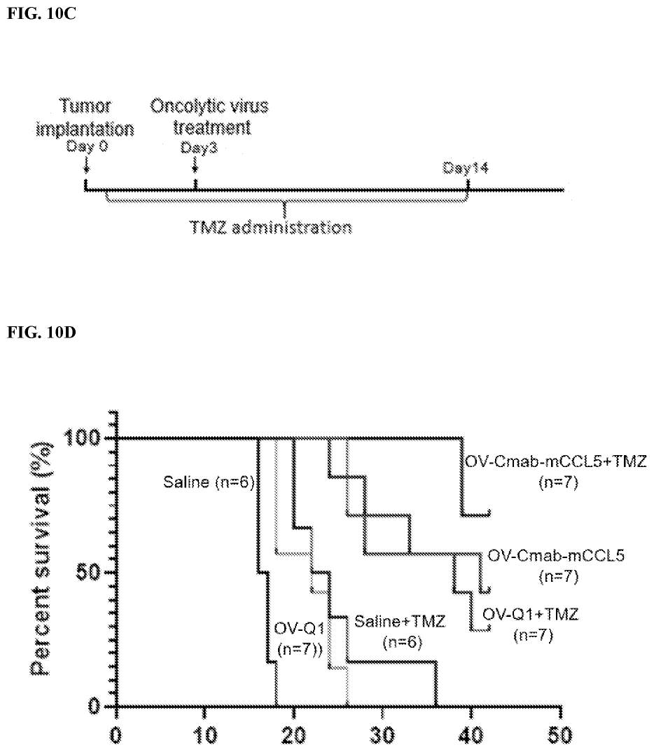

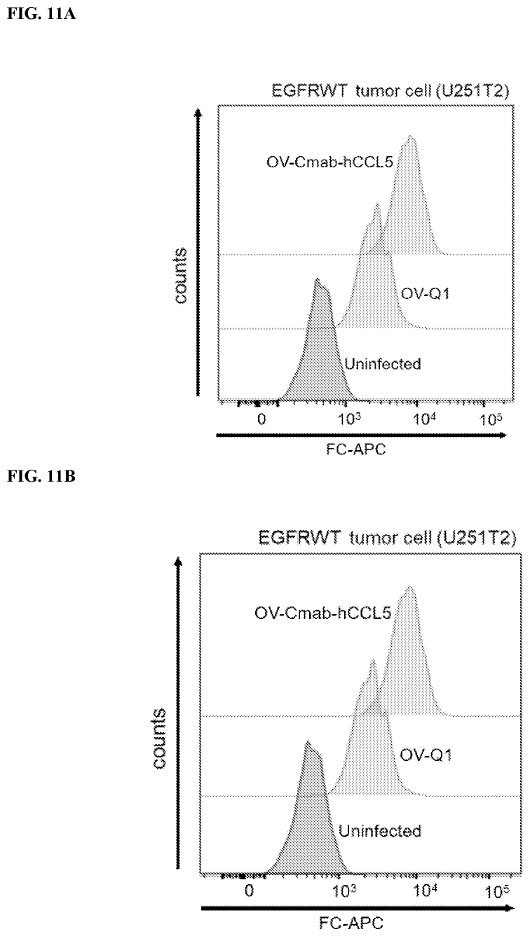

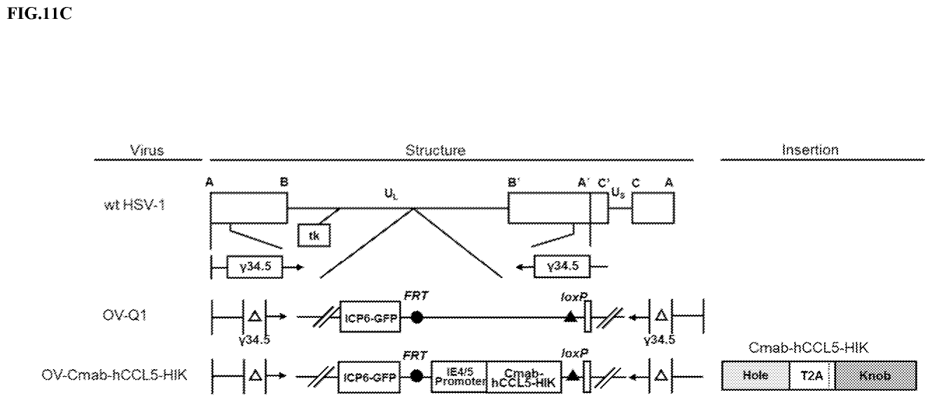

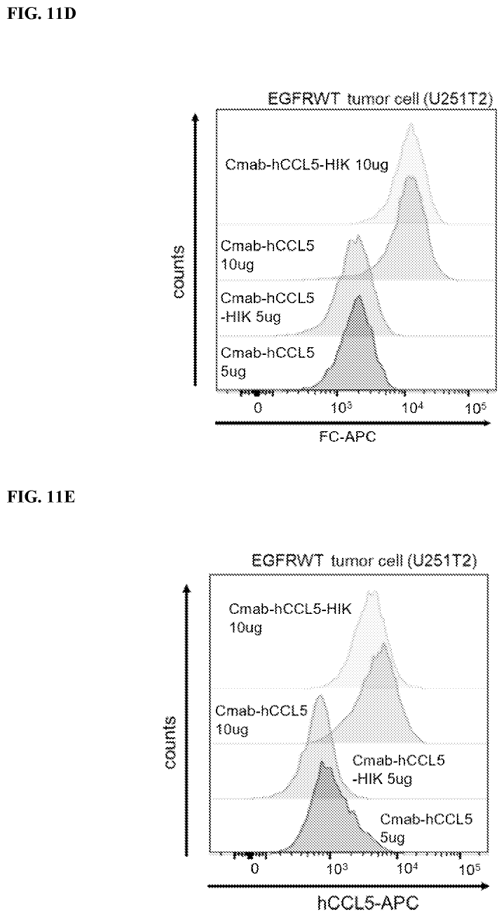

TMZ, which is an imidazotetrazine that is converted to a compound capable of alkylating DNA, interferes with DNA replication and leading to cytotoxicity in proliferating cells, and has been in clinical trials for treating GBM. To detect whether OV-Cmab-CCL5 enhanced TMZ function for treating GBM, the combination therapy with OV-Cmab-CCL5 and TMZ in vivo and in vitro was performed. Gli36ΔEGFR cells were treated with 1 μM TMZ overnight, and the pretreated cells were ⁵¹Cr-labeled and used as target cells. These cells were then incubated with 5 μg/ml Cmab-hCCL5 or vehicle for 30 min. The effector NK cells and target cells were then co-cultured at the ratios of 40:1, 20:1, and 10:1 for 4 hours and assayed for cytotoxicity. The results demonstrated that TMZ pre-treatment enhanced NK cell cytotoxicity. In addition, it was determined that when TMZ was given with Cmab-CCL5, a significantly higher degree of NK cell cytotoxicity was observed as compared to either treatment alone (FIG. 10A). This function was also detected as induced by the supernatants from OV-Cmab-hCCL5-infected Gli36ΔEGFR cells. Consistent data were collected. The combination of OV-Cmab-CCL5 with TMZ demonstrates a synergetic effect in NK cell cytotoxicity targeting GBM cells in vitro (FIG. 10B).

### Example 10: Oncolysis

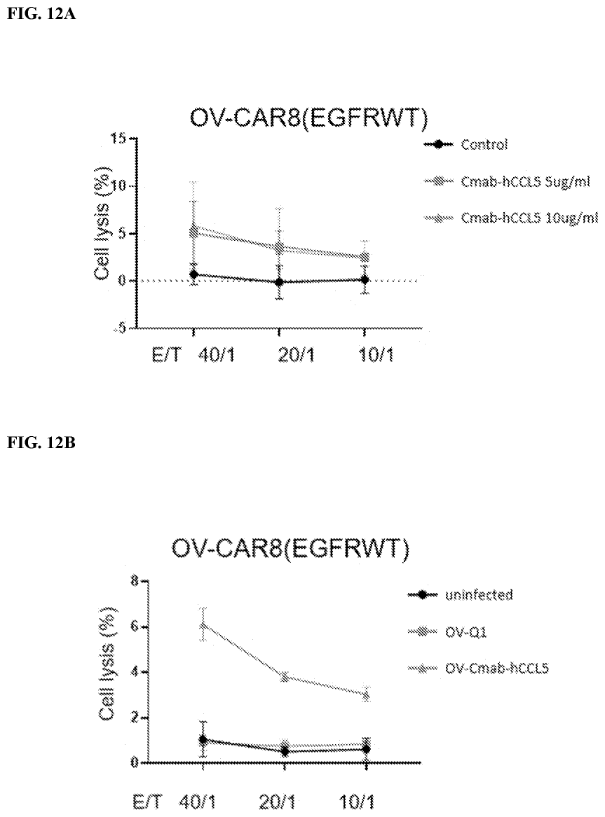

To evaluate the oncolytic effect of OV-Cmab-hCCL5 and OV-Cmab-mCCL5, the human GBM cell lines U251T2 were infected with different doses of virus. There were no difference in cell death among GBM cells infected with OV-Cmab-hCCL5, OV-Cmab-mCCL5, or OV-Q1 (FIG. 12A-12B).

### Example 11: Discussion

In this study, we constructed OV-Cmab-CCL5, an oHSV targeting both the EGFR and CCL5 receptors. OV-Cmab-CCL5 produced by infected tumor cells not only produces high levels of CCL5 in the TME, which increases infiltration of both innate and adaptive immune cells but also functions as an IgG1 anti-EGFR monoclonal antibody to activate NK cells via ADCC and macrophages via ADCP. The local delivery of OV-Cmab-CCL5 induce 1) direct tumor lysis by oHSV; 2) innate and adaptive immune cell infiltration at the TME by CCL5; 3) Cmab-mediated-ADCP by bridging Fcγ receptors on macrophages and EGFR on GBM cells; and 4) Cmab-mediated-ADCC by bridging Fcγ receptors on NK cells and EGFR on GBM cells. In vivo, OV-Cmab-CCL5 improves oncolytic virotherapy in immunodeficient xenograft and immunocompetent GBM models. Furthermore, depletion of NK cells, macrophages, or T cells impaired or abolished the protective effect of OV-Cmab-mCCL5. Thus, our novel OV provides a new compelling therapeutic candidate for patients with GBM or other EGFR⁺ cancers.

The first and only FDA-approved OV, T-VEC is being used to treat multiple cancers (22). T-VEC expresses two copies of GM-CSF driven by the strong promoter CMV in the modified HSV1 genome without γ34.5 expression (23). The GM-CSF transgenes in T-VEC may cause intense inflammation by neutrophils, monocytes or macrophages, and dendritic cells. This may subsequently lead to deleterious inflammatory processes in the brain as is seen in multiple sclerosis 24. Thus, therapies such as T-VEC, which is approved for melanoma as a single agent, might be unsuitable for treating GBM, possibly because its effects on immune activation function may be insufficient (23,25). Indeed, the combination of T-VEC with other immunomodulatory agents such as anti-PD1 antibody and anti-CTL4 antibody can significantly improve the efficacy of T-VEC (26-28). Recently, G207, with a backbone similar to OV-Cmab-CCL5, showed a safe and effective effect to treating pediatric high-grade glioma (12). Here we engineered the backbone to express an antibody-based fusion protein, OV-Cmab-CCL5, showing suitable for the treatment of GBM in experimental systems. As noted, GBM is a lethal devastating cancer with a median survival time of around 15 months (29). We designed OV-Cmab-CCL5 to enhance the immune responses of oHSV. Indeed, our data show that OV-Cmab-CCL5 not only increases immune cell infiltration but also activates various immune cells in vitro and in vivo. These effects include both innate and adaptive immune cells, and their individual depletion impaired or abolished the effect of OV-Cmab-CCL5 on improved survival of mice bearing GBM, with more profound effects for adaptive vs. innate immune cells. Furthermore, our virus could target both wtEGFR and EGFRvIII GBM cells, allowing us to target heterogeneous GBM. Importantly, our OV-Cmab-CCL5 is a single agent that achieves results seen when OV is used in combination with therapies involving immune cell modulators.

The chemokine system not only brings the antigen-presenting cells and naive T cells together to activate an adaptive immune response but also delivers both innate and adaptive immune cells to the TME (30). Among the chemokines, CCL5 is crucial for recruiting various leukocytes into inflammatory sites, including NK cells, macrophages, T cells, eosinophils, and basophils (9,31). It has been shown that CCL5 expression by tumor cells is important for the infiltration of T cells into tumors (10). CCL5 released by T cells also induces the activation and proliferation of NK cells to generate C-C chemokine-activated killer cells (31). Furthermore, CCL5 can also promote the survival of macrophages (7). However, CCL5 is often epigenetically silenced in solid tumor cells (10), unable to induce innate and adaptive immune activation targeting tumor cells. Thus, overexpressing CCL5 loco-regionally has been explored here as a promising approach for solid tumor therapy. The systemic delivery of chemokines often results in toxicities and severe side effects (32). Moreover, chemokines have very short half-lives and are challenging to deliver (33). OVs carrying chemokines can address these challenges, especially for solid tumors in situ such as GBM, because of the intra-tumoral administration. In this setting, chemokines are only enriched in the tumor site, promoting locoregional immune cell infiltration into the TME and followed by activation of the local immune response.

Over the last 15 years, monoclonal antibody (mAb)-based tumor therapies have shown remarkable success for solid tumors (34-36). Many therapeutic mAbs targeting tumor-associated antigens are approved by the FDA as immunotherapies, including cetuximab (35,37,38). Cetuximab is an EGFR inhibitor that binds the extracellular domain of EGFR or EGFRvIII and blocks tumor growth and metastasis (39,40). In preclinical GBM models, cetuximab showed an antitumor and radio-sensitizing effect on GBM (41,42). Thus, cetuximab therapy is a promising strategy for treating GBM tumors that overexpress EGFR and/or EGFRvIII. In our approach, cetuximab secreted by OV-Cmab-CCL5 infected tumor cells was continuously released into the TME to bind to GBM cells and may also block tumor cell growth.

The TME of GBM is considered immunosuppressive due to the cytokines secreted by tumor cells and tumor-associated macrophages, which inhibit both the innate and adaptive immune systems (43-45). Thus, NK cell activity is suppressed, and T cell proliferation is downregulated (46). Furthermore, standard treatments such as radiotherapy, chemotherapy, and glucocorticoid therapy are immunosuppressive and can induce immune cell death (47,48). Thus, combining standard therapy with immunotherapy has emerged as a key tool for targeting GBM. Immunotherapies, including checkpoint inhibitors, chimeric antigen receptor-T cell therapy, and oncolytic virotherapy are all under active consideration for GBM treatment (49). Our OV-Cmab-CCL5 platform has a broad application, as we can replace a part of or the entire Cmab-CCL5 with a bispecific antibody, a fusion protein, a cytokine, or any other agent to specifically change the TME from “cold’ to “hot” or further fine-tune a therapeutic approach. Compared to lentivirus or retrovirus-base gene therapy, oHSV has an advantage as the genome size of oHSV is around 150 kb, which allows oHSV to carry a large size that can include one or multiple transgenes.

T cell dysfunction, including senescence, anergy, tolerance, exhaustion, and ignorance, is a hallmark of GBM (50-52). Thus, a number of clinical trials that aim to specifically activate T-cells activation have been shown to be efficient and safe, and adoptive T cell transfer represents a promising approach for GBM therapy (53,54). In our study, OV-Cmab-CCL5 intratumoral injection promotes CD4+ and CD8+ T cell infiltration, and depletion of T cells could completely abolish the survival advantage of therapy with OV-Cmab-mCCL5. Some studies showed that the CD4/CD8 T cell ratio predicts the outcome of cancer treatment since CD8+ cells play a crucial role in antitumor immune responses (55,56). Specifically, CD8+ T cell infiltration is associated with prolonged survival in GBM patients (57). Our data show that T cell infiltration is increased while the CD4+/CD8 T cell ratio is decreased after OV-Cmab-CCL5 intra-tumoral injection, indicating that CD8+ T cells play an important role in OV-Cmab-CCL5 therapy.

In summary, we generated a novel oHSV bispecific fusion protein platform expressing cetuximab and CCL5, which combines chemokine activity with mAb function to treat GBM. This platform can deliver a chemokine in a targeted manner, as another transgene such as one encoding an antibody that can bind to tumor cells, allowing for tumor-specific targeting in the TME. Our current platform improves the efficacy of oncolytic virotherapy via combining an immunoregulatory factor with mAb therapy for tumor treatment using a single agent with multifaced functions.

### Example 12: Methods and Materials

### Cells

All mouse and human GBM cell lines, human ovarian cell lines A2780, as well as the Chinese hamster ovary (CHO) cells, were cultured in DMEM supplemented with 10% FBS, penicillin (100 U/ml), and streptomycin (100 μg/ml). CT2A-hEFGR cells were generated by transfecting CT2A cells to express the human EGFR gene. GBM30 spheroid cells, which are patient-derived, were modified to express a fly luciferase (FFL) gene for in vivo imaging and were named GBM30-FFL. Both GBM30 and GBM30-FFL cells were maintained as tumor spheres with basic neurobasal media supplemented with 2% B27, human epidermal growth factor (EGF, 20 ng/ml) and fibroblast growth factor (FGF, 20 ng/ml) in low-attachment cell culture flasks. Vero cells, which are derived from monkey kidney epithelium, were used for viral propagation and plaque-based viral titration assay and were maintained with the same media used for GBM cell lines. Gli36ΔEGFR, U87ΔEGFR, U251T2, and GBM30 cells were authenticated by the University of Arizona Genetics Core via short tandem repeat profiling in January 2015. Vero cells were not authenticated after receipt. All cell lines were routinely tested for the absence of mycoplasma using the MycoAlert Plus Mycoplasma Detection Kit from Lonza (Walkersville, MD).

### Generation and Purification of Cmab-hCCL5 and Cmab-mCCL5

Cmab-hCCL5 and Cmab-mCCL5 were purified from lentivirus-infected CHO cells. The sequence of cetuximab scFv was reconstructed as previously reported (58). CHO cells were transduced with the pCDH-CMV lentiviral vector to express Cmab-hCCL5 or Cmab-mCCL5. The “knob” and “hole”, encoding Cmab-Fc and CCL5-Fc, respectively, were linked with a DNA sequence encoding a T2A self-cleaving peptide to express them simultaneously. The sequence was carried by pCDH-CMV plasmid with a GFP selection marker for sorting the GFP-positive CHO cells using a FACS Aria II cell sorter (BD Biosciences, San Jose, CA, USA). Cmab-hCCL5 and Cmab-mCCL5 were purified from the conditional supernatants of the lentivirus-infected CHO cells using a protein G column (Thermo Fisher, cat #89927).

### EGFR Binding Assays and CCL5 Expression Assay

U251T2, U87ΔEGFR, and A4780 cells were pre-blocked with 2% BSA and incubated with 1, 5, or 10 μg/ml purified Cmab-hCCL5 or 1, 5, or 10 μg/ml human IgG1 isotype control for 30 min. Murine CT2A-hEGFR cells were pre-blocked with 2% BSA and then incubated with 1, 5, or 10 μg/ml purified Cmab-mCCL5 or 1, 5, or 10 μg/ml human IgG1 isotype control for 30 min. After incubation, all cells were washed twice, and human cell lines were stained with APC-conjugated anti-human Fc (Jackson ImmunoResearch, cat #209-605-098) or APC-conjugated anti-human CCL5 (Biolegend, cat #515506, 149103) antibodies, whereas the murine CT2A-hEGFR cells were stained with APC-conjugated anti-human Fc or PE-conjugated anti-mouse CCL5 antibodies, for 20 min. The stained cells were analyzed using a Fortessa X20 flow cytometer (BD Biosciences).

### Generation of OV-Cmab-hCCL5 and OV-Cmab-mCCL5

OV-Cmab-hCCL5 and OV-Cmab-mCCL5 were generated by using the fHsvQuik-1 system, as previously described (13,59). The “knob” and “hole”, encoding Cmab-Fc and CCL5-Fc, respectively, were linked with a DNA sequence encoding a T2A self-cleaving peptide to express them simultaneously. The Cmab-Fc-T2A-hCCL5-Fc and the Cmab-Fc-T2A-mCCL5-Fc sequences were inserted into pT-oriSIE4/5 downstream of the HSV pIE4/5 promoter to construct the pT-oriSIE4/5-Cmab-hCCL5 and pT-oriSIE4/5-Cmab-mCCL5 transfer plasmids. The two transfer plasmids were recombined using fHlsvQuik-1 to engineer OV-Cmab-hCCL5 and OV-Cmab-mCCL5. Vero cells were used for propagating and titrating the viruses. Virus titration was performed using plaque assays, for which monolayer Vero cells were seeded in a 96-well plate. Twelve hours later, the seeded cells were infected with gradient-diluted viral solutions. Two hours post infection, the infection media was replaced with DMEM supplemented with 10% FBS. GFP-positive plaques were observed and counted with a Zeiss fluorescence microscope (AXIO observer 7) 2 days after infection to calculate the viral titer. To concentrate and purify the OV-Q1, OV-Cmab-hCCL5 and OV-Cmab-mCCL5 viral particles, we harvested the culture media containing viruses and centrifuged them at 3,000×g for 30 min. The supernatants of the 3,000×g centrifugation were collected and ultra-centrifuged at 100,000×g for 1 hour. The virus pellets were resuspended with saline as needed.

### Immunoblotting Assay

The immunoblotting assay was performed as previously described (13). U251T2 GBM cells were infected with OV-Q1, OV-Cmab-hCCL5, or OV-Cmab-mCCL5 at an MOI of 2. The infection media was replaced with fresh media after a two-hour infection. The supernatants from each group were harvested at 48 hpi to extract protein by a chloroform-methanol method for immunoblotting. The Cmab-hCCL5 and Cmab-mCCL5 fusion proteins extracted from lentivirus-infected CHO cells using the same method were included as controls. A rabbit anti-human IgG heavy chain antibody (Sigma, cat #MAB1307), a rabbit anti-human CCL5 antibody with cross reactivity with mouse CCL5 (Abcam, Cat #ab10394), and an anti-rabbit secondary antibody (LI-COR, 925-32210) were used to detect the Fc and CCL5, respectively, in the Cmab-hCCL5 or Cmab-mCCL5 fusion protein.

### Measurement of Cmab-hCCL5 and Cmab-mCCL5 Concentration by ELISA

U251T2 GBM cells were infected with OV-Q1, Cmab-hCCL5 or Cmab-mCCL5 at a multiplicity of infection of 2. Two hours after the infection, the infection media was discarded and replaced with fresh media. The supernatants from each group were then harvested at 12, 24, 48, and 72 hpi to measure the Cmab-hCCL5 or Cmab-mCCL5 concentration by ELISA. Cmab-hCCL5 or Cmab-mCCL5 samples with known concentrations, purified from CHO cells by protein G columns, were used as standards. The ELISA for measuring Cmab-hCCL5 concentration using an anti-Fc antibody was performed as previously reported with slight modification (60). Briefly, recombinant human EGFR protein (Abcam, cat #ab174029) was used as a coating reagent. Anti-human Fc antibody (Sigma, cat #MAB1307) was used to detect Fc within the Cmab-Fc fusion protein. Commercial human or mouse CCL5 Quantikine ELISA Kits (R&D, cat #DRN00B, MMR00, respectively) were used to detect hCCL5 or mCCL5 within the Cmab-hCCL5 or Cmab-mCCL5 fusion protein, respectively, via the interaction between a CCL5 antibody and the CCL5 part of Cmab-hCCL5 or Cmab-mCCL5.

### Preparation of Immune Cells for In Vitro Studies

To isolate primary human macrophages, T cells, and NK cells, we isolated peripheral blood mononuclear cells (PBMCs) from peripheral blood of healthy donors. Human monocytes were isolated and enriched by using the RosetteSep™ Human Monocyte Enrichment Cocktail kit (Stemcell, cat #15068) from the peripheral blood. Human NK cells were isolated and enriched using the RosetteSep™ Human NK Enrichment Cocktail kit (Stemcell, cat #15065), and the RosetteSep™ Human T Cell Enrichment Cocktail (Stemcell, cat #15061) was used to isolate and enrich human T cells from peripheral blood mononuclear cells. The enriched human monocytes were cultured with RPMI-1640 media containing 20 ng/ml human M-CSF (PeproTech, Cat #300-25-50 UG) and 2% human serum for 7 days to induce macrophage differentiation. The culture media was replaced on Day 3 and Day 5). Dynabeads™ Human T-Activator CD3/CD28 for T Cell Expansion and Activation (Thermo, cat #11132D) beads were used to activate human T cells for 3 days with RPMI-1640 media containing 20% FBS, 100 U/ml penicillin/streptomycin, and 10 ng/ml IL-2. Human NK cells were used immediately for in vitro migration and cytotoxicity assays.

For isolating and culturing mouse macrophages, C57BL/6J mice were sacrificed at the time of bone marrow harvest. Bone marrow cells were extracted from the tibias and femurs by flushing with culture media using a 25 G needle. The cells were then passed through a 70 μm nylon mesh (BD Bioscience) and washed three times with PBS. Red blood cells were lysed with RBC Lysis Buffer (Thermo Fisher, cat #00-4300-54). Extracted BM cells (2.4×10⁷) were planted in a 100 mm culture dish (BD Falcon) and cultured for 7 days with RPMI-1640 media containing 10% FBS in the presence of 20 ng/ml murine M-CSF (PeproTech, cat #315-02). The culture media was replaced on Day 3 and Day 5). The EasySep™ Mouse NK Cell Isolation Kit (Stemcell, cat #19855) and EasySep™ Mouse T Cell Isolation Kit (Stemcell, cat #19851) were used to isolate mouse NK cells and T cells from the spleens of C57BL/6J mice. Freshly isolated NK cells and T cells were used immediately for in vitro migration and cytotoxicity assays.

### Cell Migration Assay

The migration capability of NK cells, T cells, and macrophages was determined using transwell chambers (Corning). Serum-starved NK cells, T cells or macrophages (10⁶/well) were seeded to an upper chamber. Recombinant human (rh) CCL5, recombinant mouse (rm) CCL5, purified Cmab-hCCL5 or Cmab-mCCL5, or the supernatant containing Cmab-hCCL5 or Cmab-mCCL5 secreted from OV-Cmab-hCCL5- or OV-Cmab-mCCL5-infected U251T2 GBM cells was added to the lower chamber. The chambers were incubated at 37° C. in 5% CO₂ for 12 h. Cells that migrated to the lower face of the porous membrane in the lower chamber were counted. For the T cells and NK cells, the pore size of the transwell membrane used in this assay was 3 μm as previously described (61,62). For the macrophages, the pore size of the transwell membrane used in this assay was 8 m as previously described (63).

### NK Cell Cytotoxicity and Activation Assay

NK cell cytotoxicity was evaluated as previously described (13,64,65). Briefly, U251T2, GBM30, Gli36ΔEGFR, and CT2A-hEGFR cells were used as target cells. The target cells were labeled with ⁵¹Cr for 1 h, followed by incubation with 5 μg/ml or 10 μg/ml of the Cmab-hCCL5 fusion protein or IgG1 isotype control for 30 min. The labeled target cells preincubated with the Cmab-hCCL5 fusion protein or IgG1 were co-cultured with isolated human primary NK cells at different effector:target ratios at 37° C. for 4 h. Release of ⁵¹Cr was measured as counts per minute (cpm) with a MicroBeta(2) microplate radiometric counter (Perkin Elmer, Waltham, MA). Target cells incubated in complete media were for spontaneous release controls and in 1% SDS media were for maximal ⁵¹Cr release controls. The cell lysis percentages were calculated using the standard formula: 100×(cpm experimental release−cpm spontaneous release)/(cpm maximal release−cpm spontaneous release). The assays were performed with at least three technical replicates using NK cells from different donors. The expression of CD69 and granzyme B, NK cell activation markers, were measured after 6-hour co-culture of NK cells and GBM30 cells with 5 μg/ml or 10 μg/ml of Cmab-hCCL5 at a ratio of 1:1 with a flow cytometer after the cells were stained with the anti-CD56 (BD, cat #557919) and anti-CD69 (BD, cat #562883) or anti-granzyme B (BD, cat #563388) antibodies. Following similar procedures, mouse CD69 was measured by an anti-CD69 (BD, cat #564683) antibody, and NK cells were marked by an anti-NKp46 (Biolegend, cat #137618) antibody.

### Flow Cytometry-Based Phagocytosis Assay

For the phagocytosis assay of primary human macrophages, GBM30 cells stained with CFSE (Thermo Fisher, cat #C34554) were used as target cells. Human macrophages and target cells were co-cultured at a ratio of 1:2 for 6 hours with 5 μg/ml or 10 μg/ml of the Cmab-hCCL5 fusion protein or IgG1 isotype control in a humidified, 5% CO2 incubator at 37° C. in ultra-low-attachment 96-well U-bottom plates (Corning) in serum-free 1640 media (Life Technologies). The co-cultured cells were harvested by centrifuging at 400×g for 5 min at 4° C. and stained with an anti-human CD45 antibody (Biolegend, cat #368516) to identify macrophages. All flow cytometry data were collected using a Fortessa X20 flow cytometer (BD Biosciences). Phagocytosis was measured as the number of CD45⁺CFSE⁺ macrophages, quantified as a percentage of the total CD45⁺ cells.

For the phagocytosis of mouse macrophages, CT2A-hEGFR cells stained with CFSE were used as target cells. Murine macrophages and target cells were co-cultured at a ratio of 1:2 for 6 hours with 5 μg/ml or 10 μg/ml Cmab-mCCL5 or IgG1 isotype, in a humidified, 5% CO2 incubator at 37° C. in ultra-low-attachment 96-well U-bottom plates (Corning) in serum-free 1640 (Life Technologies). Then the cells were harvested by centrifuging at 400×g for 5 min at 4° C. and stained with an anti-mouse F4/80 antibody (BD, cat #565787) to identify macrophages. Phagocytosis was measured as the number of F4/80⁺CFSE⁺ macrophages and quantified as a percentage of the total F4/80 macrophages.

### Quantitative PCR

For evaluating the effect of Cmab-hCCL5 on mRNA levels of typical human macrophage cytokine genes, human macrophages and GBM30 cells were co-cultured at a ratio of 1:1 for 6 hours with or without 5 μg/ml Cmab-hCCL5. The macrophages were stained with anti-human CD45 antibody and sorted using an Aria Fusion flow cytometer (BD Biosciences). The total RNA was extracted from the sorted macrophages for measuring the mRNA levels of human IL1B, IL6, IL12A, and NOS2 genes with their corresponding primers. 18s rRNA was used as an internal control.

For evaluating the effect of Cmab-mCCL5 on the mRNA levels of typical mouse macrophage genes, mouse macrophages and CT2A-hEGFR cells were co-cultured at a ratio of 1:1 for 6 hours with or without 5 μg/ml Cmab-mCCL5. Murine macrophages were stained with an anti-mouse F4/80 antibody and sorted using an Aria Fusion flow cytometer. Total RNA was extracted from the sorted mouse macrophages for measuring the mRNA levels of murine Il1b, Il6, Il12b, Ccl-2, Ccl-4, and Nos2 genes with their corresponding primers. 18s rRNA was used as an internal control (Table 1).

### Animal Studies

For establishing an immunodeficient xenograft GBM model, 6-8-week-old female NOD.Cg-Prkd^{scid} Il2rγ^{tm1Wj1}/SzJ (NSG) mice were purchased from Jackson Laboratories (Bar Harbor, Maine). For the survival studies, mice were anesthetized and stereotactically injected with 1×10⁵ GBM30-FFL cells, into the right frontal lobe of the brain (2 mm lateral and 1 mm anterior to bregma at a depth of 3 mm). 4 days after tumor implantation, 1×10⁶ peripheral blood mononuclear cells (PBMCs) isolated from a single donor to each mouse was intravenously injected. On Day 6, 1×10⁶ activated T cells derived from the same donor were delivered to each mouse by intravenous injection. On Day 7, animals were subsequently randomly divided into groups that were intracranially injected either with 2×10⁵ pfu oHSV (OV-Q1 or OV-Cmab-hCCL5) in 3 μl of saline or with saline as control. The mice were subsequently monitored and weighed frequently to evaluate GBM disease progression. Luciferase-based in vivo images were taken 15 days after tumor implantation to assess the tumor development. The mice were euthanized when they became moribund, with neurologic impairments and obvious weight loss.

For establishing an immunocompetent mouse GBM model, 6-8-week-old C57BL/6J mice were purchased from Jackson Laboratories. 1×10⁵ CT2A-hEGFR cells were injected as described for the immunodeficient xenograft model. For the survival studies, the cells grew for 3 days, and animals were subsequently randomly divided into groups that were intracranially injected either with 2×10⁵ pfu oHSV (OV-Q1 or OV-Cmab-mCCL5) in 3 l of saline or with saline as a control. The mice were euthanized when they became moribund, with neurologic impairments and obvious weight loss. For the in vivo immunocyte infiltration study, the cells grew for 5 d, and animals were subsequently randomly divided into groups that were i.e. injected either with 2×10⁵ pfu oHSV (OV-Q1 or OV-Cmab-mCCL5) in 3 μl of saline or with saline as control. The mice were euthanized on Day 7 after tumor implantation to evaluate immunocyte brain infiltration by flow cytometry as described below.

The survival studies involving immune cell depletions were performed using the same CT2A-hEGFR GBM model mentioned above. NK cells were depleted by i.p. injections of NK1.1 neutralizing antibody (BioXcell, BE0036, clone PK136; 0.2 mg/mouse) twice, once on the day before virus injection and once 2 days after the virus injection. Macrophages were depleted by i.p. injections of clodronate liposomes (0.2 ml/mouse) using the same schedule as the NK cell depletion studies. CD4+ and CD8+ T cells were depleted by three i.p. injections of anti-CD4 (BioXcell, BP0003-1, clone GK1.5; 0.2 mg/mouse) and anti-CD8 neutralizing antibodies (BioXcell, BE0004-1, clone 53-6.7; 0.2 mg/mouse, once every 3 days beginning the day before virus injection. The corresponding isotypes and liposomes were used as controls.

For establishing a bilateral CT2A-hEGFR immunocompetent GBM model, 6-8-week-old C57BL/6J mice were purchased from Jackson Laboratories. Mice were anesthetized and stereotactically injected with 1×10⁵ CT2A-hEGFR cells into both the left and right frontal lobe of the brain (2 mm lateral and 1 mm anterior to bregma at a depth of 3 mm). The cells grew for 2 days, and animals were subsequently randomly divided into groups that were intracranially injected with either 2×10⁵ pfu oHSV (OV-Q1 or OV-Cmab-mCCL5) in 3 μl of saline or with saline as control only in the right hemisphere of the brain. MRI was used to monitor the tumor size on Day 9.

### Magnetic Resonance Imaging (MRI)

MRI imaging was performed on a 7T Benchtop MRI system (Bruker Corporation) Multislice T2-weighted spin-echo (echo time=84.0 ms; repetition time=2500 ms; field of view (FOV)=20 mm; matrix=160×160; 5 slices; slice thickness=1 mm; total imaging time=20 min).

### Flow Cytometry

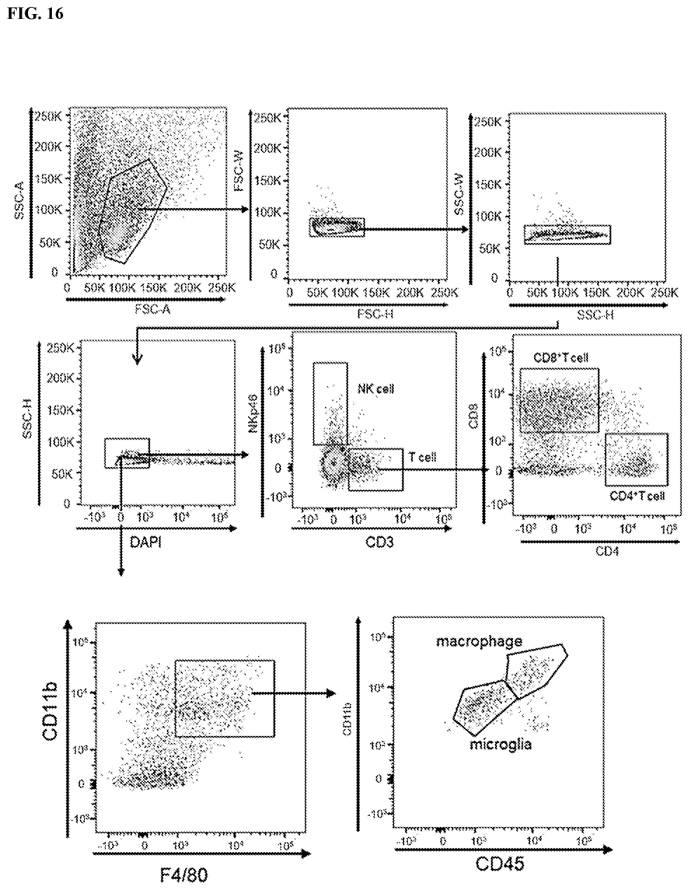

Mononuclear cells in the brain were extracted with Percoll and stained with anti-NKp46 (Biolegend, cat #137618), anti-CD3 (BD, cat #553066), anti-CD45 (BD, cat #559864), anti-CD11b (BD, cat #552850), anti-F4/80 (Thermo, cat #12-4801-82), anti-CD4 (BD, cat #557308) and anti-CD8 (BD, cat #553030) antibodies for flow cytometric analysis of immune cell brain infiltration. The flow cytometric assessments of murine immune cells were performed with at least 5 independent animals. All flow cytometry data were collected using the Fortessa X-20 flow cytometer except cell sorting experiments that were performed using FACS Aria II cell sorter (BD Biosciences). Gating strategies were shown in FIG. 16.

### Oncolysis Assay

U251T2 GBM cells were harvested, and individual cell suspensions were prepared at a cell density of 2×10⁵ cells/ml. U251T2 GBM cells were then seeded into a special 96-well E-plate (ACEA Biosciences) at 100 μl/well and incubated at 37° C. to allow cell attachment and formation of a cell monolayer. Twelve hours later, different MOIs of OV-Q1 and OV-Cmab-hCCL5 were added to the plate. The oncolysis assay was monitored and analyzed with the real-time cell analysis (RTCA) software (ACEA Biosciences).

### Statistical Analysis

For continuous endpoints that are normally distributed or logarithm transformed data, data are presented as mean±SD. Student's t test was used to compare two independent conditions, and one-way ANOVA was used to compare three or more conditions. For data with repeated measures from the same subject/donor, a linear mixed model was used for group comparisons by accounting for the underlying variance and covariance structure. For survival data, Kaplan-Meier method and log-rank test were used to estimate and compare survival functions. Cox regression model was used to test the interaction between OV-Cmab-mCCL5 and NK cell/macrophage/T cell depletion, or in other words, the comparison between the difference of OV-Cmab-mCCL5 with vs. without immune cell depletion and the difference OV-Q1 with vs. without immune cell depletion. All tests were two-sided. P values were adjusted for multiple comparisons by Holm's method or B-H method. A P value of 0.05 or less was defined as statistically significant. Statistical software GraphPad, R.3.6.3. and SAS 9.4 were used for the statistical analysis.

### REFERENCES

1. Siegel, R. L., Miller, K. D. & Jemal, A. Cancer statistics, 2018. CA Cancer J Clin 68, 7-30 (2018).
2. Ladomersky, E., et al. The Coincidence Between Increasing Age, Immunosuppression, and the Incidence of Patients With Glioblastoma. Front Pharmacol 10, 200 (2019).
3. Nduom, E. K., Weller, M. & Heimberger, A. B. Immunosuppressive mechanisms in glioblastoma. Neuro Oncol 17 Suppl 7, vii9-vii14 (2015).
4. Ooi, Y. C., et al. The role of regulatory T-cells in glioma immunology. Clin Neurol Neurosurg 119, 125-132 (2014).
5. Calinescu, A. A., et al. Overview of current immunotherapeutic strategies for glioma. Immunotherapy 7, 1073-1104 (2015).
6. Perng, P. & Lim, M. Immunosuppressive Mechanisms of Malignant Gliomas: Parallels at Non-CNS Sites. Front Oncol 5, 153 (2015).
7. Keophiphath, M., Rouault, C., Divoux, A., Clement, K. & Lacasa, D. CCL5 promotes macrophage recruitment and survival in human adipose tissue. Arterioscler Thromb Vasc Biol 30, 39-45 (2010).
8. Khalid, A., et al. Recent Advances in Discovering the Role of CCL5 in Metastatic Breast Cancer. Mini Rev Med Chem 15, 1063-1072 (2015).
9. Aldinucci, D. & Colombatti, A. The inflammatory chemokine CCL5 and cancer progression. Mediators Inflamm 2014, 292376 (2014).
10. Dangaj, D., et al. Cooperation between Constitutive and Inducible Chemokines Enables T Cell Engraftment and Immune Attack in Solid Tumors. Cancer Cell 35, 885-900 e810 (2019).
11. Ma, W., He, H. & Wang, H. Oncolytic herpes simplex virus and immunotherapy. BMC Immunol 19, 40 (2018).
12. Friedman, G. K., et al. Oncolytic HSV-1 G207 Immunovirotherapy for Pediatric High-Grade Gliomas. N Engl J Med (2021).
13. Xu, B., et al. An oncolytic herpesvirus expressing E-cadherin improves survival in mouse models of glioblastoma. Nat Biotechnol (2018).
14. Han, J., et al. TGFbeta Treatment Enhances Glioblastoma Virotherapy by Inhibiting the Innate Immune Response. Cancer Res 75, 5273-5282 (2015).
15. Chen, X., et al. A combinational therapy of EGFR-CAR NK cells and oncolytic herpes simplex virus 1 for breast cancer brain metastases. Oncotarget 7, 27764-27777 (2016).
16. Renner, C., et al. Cure of xenografted human tumors by bispecific monoclonal antibodies and human T cells. Science 264, 833-835 (1994).
17. Krishnamurthy, A. & Jimeno, A. Bispecific antibodies for cancer therapy: A review. Pharmacol Ther 185, 122-134 (2018).
18. Kontermann, R. E. & Brinkmann, U. Bispecific antibodies. Drug Discov Today 20, 838-847 (2015).
19. Wei, H., et al. Structural basis of a novel heterodimeric Fc for bispecific antibody production. Oncotarget 8, 51037-51049 (2017).
20. Liu, B. L., et al. ICP34.5 deleted herpes simplex virus with enhanced oncolytic, immune stimulating, and anti-tumour properties. Gene Ther 10, 292-303 (2003).
21. Kitange, G. J., et al. Induction of MGMT expression is associated with temozolomide resistance in glioblastoma xenografts. Neuro Oncol 11, 281-291 (2009).
22. Rehman, H., Silk, A. W., Kane, M. P. & Kaufman, H. L. Into the clinic: Talimogene laherparepvec (T-VEC), a first-in-class intratumoral oncolytic viral therapy. J Immunother Cancer 4, 53 (2016).
23. Conry, R. M., Westbrook, B., McKee, S. & Norwood, T. G. Talimogene laherparepvec: First in class oncolytic virotherapy. Hum Vaccin Immunother 14, 839-846 (2018).
24. Hamilton, J. A. GM-CSF in inflammation and autoimmunity. Trends Immunol 23, 403-408 (2002).
25. Stupp, R., et al. Effects of radiotherapy with concomitant and adjuvant temozolomide versus radiotherapy alone on survival in glioblastoma in a randomised phase III study: 5-year analysis of the EORTC-NCIC trial. Lancet Oncol 10, 459-466 (2009).
26. Kelly, C. M., et al. Objective Response Rate Among Patients With Locally Advanced or Metastatic Sarcoma Treated With Talimogene Laherparepvec in Combination With Pembrolizumab: A Phase 2 Clinical Trial. JAMA Oncol 6, 402-408 (2020).
27. Ribas, A., et al. Oncolytic Virotherapy Promotes Intratumoral T Cell Infiltration and Improves Anti-PD-1 Immunotherapy. Cell 174, 1031-1032 (2018).
28. Puzanov, I., et al. Talimogene Laherparepvec in Combination With Ipilimumab in Previously Untreated, Unresectable Stage IIIB-IV Melanoma. J Clin Oncol 34, 2619-2626 (2016).
29. Raman, S. S., Hecht, J. R. & Chan, E. Talimogene laherparepvec: review of its mechanism of action and clinical efficacy and safety. Immunotherapy 11, 705-723 (2019).
30. Mollica Poeta, V., Massara, M., Capucetti, A. & Bonecchi, R. Chemokines and Chemokine Receptors: New Targets for Cancer Immunotherapy. Front Immunol 10, 379 (2019).
31. Soria, G. & Ben-Baruch, A. The inflammatory chemokines CCL2 and CCL5 in breast cancer. Cancer Lett 267, 271-285 (2008).
32. Chada, S., Ramesh, R. & Mhashilkar, A. M. Cytokine- and chemokine-based gene therapy for cancer. Curr Opin Mol Ther 5, 463-474 (2003).
33. Whiteside, T. L. Cytokines and cytokine measurements in a clinical laboratory. Clin Diagn Lab Immunol 1, 257-260 (1994).
34. Elgundi, Z., Reslan, M., Cruz, E., Sifniotis, V. & Kayser, V. The state-of-play and future of antibody therapeutics. Adv Drug Deliv Rev 122, 2-19 (2017).
35. Scott, A. M., Wolchok, J. D. & Old, L. J. Antibody therapy of cancer. Nat Rev Cancer 12, 278-287 (2012).
36. Adams, G. P. & Weiner, L. M. Monoclonal antibody therapy of cancer. Nat Biotechnol 23, 1147-1157 (2005).
37. Reichert, J. M., Rosensweig, C. J., Faden, L. B. & Dewitz, M. C. Monoclonal antibody successes in the clinic. Nat Biotechnol 23, 1073-1078 (2005).
38. Melero, I., Hervas-Stubbs, S., Glennie, M., Pardoll, D. M. & Chen, L. Immunostimulatory monoclonal antibodies for cancer therapy. Nat Rev Cancer 7, 95-106 (2007).
39. Jonker, D. J., et al. Cetuximab for the treatment of colorectal cancer. N Engl J Med 357, 2040-2048 (2007).
40. Graham, J., Muhsin, M. & Kirkpatrick, P. Cetuximab. Nat Rev Drug Discov 3, 549-550 (2004).
41. Lustig, R. Long term responses with cetuximab therapy in glioblastoma multiforme. Cancer Biol Ther 5, 1242-1243 (2006).
42. Cho, J., et al. Glioblastoma-derived epidermal growth factor receptor carboxyl-terminal deletion mutants are transforming and are sensitive to EGFR-directed therapies. Cancer Res 71, 7587-7596 (2011).
43. Chen, Z. & Hambardzumyan, D. Immune Microenvironment in Glioblastoma Subtypes. Front Immunol 9, 1004 (2018).
44. Wang, Q., et al. Tumor Evolution of Glioma-Intrinsic Gene Expression Subtypes Associates with Immunological Changes in the Microenvironment. Cancer Cell 32, 42-56 e46 (2017).
45. Martinez-Lage, M., et al. Immune landscapes associated with different glioblastoma molecular subtypes. Acta Neuropathol Commun 7, 203 (2019).
46. Zhang, B., Shen, R., Cheng, S. & Feng, L. Immune microenvironments differ in immune characteristics and outcome of glioblastoma multiforme. Cancer Med 8, 2897-2907 (2019).
47. Harris, J., Sengar, D., Stewart, T. & Hyslop, D. The effect of immunosuppressive chemotherapy on immune function in patients with malignant disease. Cancer 37, 1058-1069 (1976).
48. Dix, A. R., Brooks, W. H., Roszman, T. L. & Morford, L. A. Immune defects observed in patients with primary malignant brain tumors. J Neuroimmunol 100, 216-232 (1999).
49. Iorgulescu, J. B., Reardon, D. A., Chiocca, E. A. & Wu, C. J. Immunotherapy for glioblastoma: going viral. Nat Med 24, 1094-1096 (2018).
50. Woroniecka, K. I., Rhodin, K. E., Chongsathidkiet, P., Keith, K. A. & Fecci, P. E. T-cell Dysfunction in Glioblastoma: Applying a New Framework. Clin Cancer Res 24, 3792-3802 (2018).
51. Woroniecka, K., et al. T-Cell Exhaustion Signatures Vary with Tumor Type and Are Severe in Glioblastoma. Clin Cancer Res 24, 4175-4186 (2018).
52. Mirzaei, R., Sarkar, S. & Yong, V. W. T Cell Exhaustion in Glioblastoma: Intricacies of Immune Checkpoints. Trends Immunol 38, 104-115 (2017).
53. Schuessler, A., et al. Autologous T-cell therapy for cytomegalovirus as a consolidative treatment for recurrent glioblastoma. Cancer Res 74, 3466-3476 (2014).
54. Brown, C. E., et al. Regression of Glioblastoma after Chimeric Antigen Receptor T-Cell Therapy. N Engl J Med 375, 2561-2569 (2016).
55. Shah, W., et al. A reversed CD4/CD8 ratio of tumor-infiltrating lymphocytes and a high percentage of CD4(+)FOXP3(+) regulatory T cells are significantly associated with clinical outcome in squamous cell carcinoma of the cervix. Cell Mol Immunol 8, 59-66 (2011).
56. Huang, Y., et al. CD4+ and CD8+ T cells have opposing roles in breast cancer progression and outcome. Oncotarget 6, 17462-17478 (2015).
57. Yang, I., et al. CD8+ T-cell infiltrate in newly diagnosed glioblastoma is associated with long-term survival. J Clin Neurosci 17, 1381-1385 (2010).
58. Ayoub, D., et al. Correct primary structure assessment and extensive glyco-profiling of cetuximab by a combination of intact, middle-up, middle-down and bottom-up ESI and MALDI mass spectrometry techniques. MAbs 5, 699-710 (2013).
59. Terada, K., Wakimoto, H., Tyminski, E., Chiocca, E. A. & Saeki, Y. Development of a rapid method to generate multiple oncolytic HSV vectors and their in vivo evaluation using syngeneic mouse tumor models. Gene Ther 13, 705-714 (2006).
60. Passaro, C., et al. Arming an Oncolytic Herpes Simplex Virus Type 1 with a Single-chain Fragment Variable Antibody against PD-1 for Experimental Glioblastoma Therapy. Clin Cancer Res 25, 290-299 (2019).
61. Gismondi, A., et al. Proline-rich tyrosine kinase 2 and Rac activation by chemokine and integrin receptors controls NK cell transendothelial migration. J Immunol 170, 3065-3073 (2003).
62. Huang, Y., et al. CRK proteins selectively regulate T cell migration into inflamed tissues. J Clin Invest 125, 1019-1032 (2015).
63. Han, Z., et al. 15-deoxy-Delta12,14-prostaglandin J2 reduces recruitment of bone marrow-derived monocyte/macrophages in chronic liver injury in mice. Hepatology 56, 350-360 (2012).
64. Wang, Y., et al. The IL-15-AKT-XBPls signaling pathway contributes to effector functions and survival in human NK cells. Nat Immunol 20, 10-17 (2019).
65. Dong, W., et al. The Mechanism of Anti-PD-L1 Antibody Efficacy against PD-L1-Negative Tumors Identifies NK Cells Expressing PD-L1 as a Cytolytic Effector. Cancer Discov 9, 1422-1437 (2019).

1. A recombinant protein comprising:
a. a first Fc fusion protein comprising a first Fc domain covalently attached to a chemokine domain; and
b. a second Fc fusion protein comprising a second Fc domain covalently attached to an anticancer therapeutic antibody domain,
wherein the first Fc domain is capable of binding to the second Fc domain.

2. (canceled)

3. The recombinant protein of claim 1, wherein the first IgG Fc domain is a first IgG1 Fc domain and the second IgG Fc domain is a second IgG1 Fc domain.

4.-9. (canceled)

10. The recombinant protein of claim 1, wherein the chemokine domain is a CC type chemokine domain, a CXC type chemokine domain, a C type chemokine domain, or a CX3C type chemokine domain.

11. (canceled)

12. The recombinant protein of claim 10, wherein the chemokine domain is a CCL5 domain.

13. (canceled)

14. The recombinant protein of claim 1, wherein the anticancer therapeutic antibody domain is an anti-EGFR antibody domain or an anti-Her2 antibody domain.

15. (canceled)

16. (canceled)

17. A nucleic acid encoding the recombinant protein of claim 1.

18. A oncolytic virus comprising a nucleic acid encoding a recombinant protein comprising:
a. a first Fc fusion protein comprising a first Fc domain covalently attached to a chemokine domain; and
b. a second Fc fusion protein comprising a second Fc domain covalently attached to an anticancer therapeutic antibody domain,
wherein the first Fc domain is capable of binding to the second Fc domain.

19. The oncolytic virus of claim 18, wherein said nucleic acid comprises an expression cassette.

20. (canceled)

21. The oncolytic virus of claim 18, wherein the first IgG Fc domain is a first IgG1 Fc domain and the second IgG Fc domain is a second IgG1 Fc domain.

22. (canceled)

23. (canceled)

24. The oncolytic virus of claim 18, wherein the oncolytic virus is an adenovirus, a herpes simplex virus, a maraba virus, a measles virus, a Newcastle virus, a picornavirus, a reovirus, a vaccinia virus, a vesicular stomatitis virus, a rubeloa virus, or a myxoma virus.

25.-27. (canceled)

28. The oncolytic virus of claim 18, wherein the chemokine is a CC type chemokine domain, a CXC type chemokine domain, a C type chemokine domain, or a CX3C type chemokine domain.

29. (canceled)

30. (canceled)

31. The oncolytic virus of claim 18, wherein the anticancer therapeutic antibody domain is an anti-EGFR antibody domain or an anti-Her2 antibody domain.

32. (canceled)

33. (canceled)

34. The oncolytic virus of claim 18, wherein the nucleic acid encoding the recombinant protein comprises a viral promoter or a tumor specific promoter.

35.-37. (canceled)

38. A pharmaceutical composition comprising the oncolytic virus of claim 18, and a pharmaceutically acceptable excipient.

39. A method of killing a cancer cell, said method comprising contacting said cancer cell with the oncolytic virus of claim 18.

40. The method of claim 39, further comprising contacting said cancer cell with a plurality of immune cells.

41.-43. (canceled)

44. A method of treating or preventing cancer in a subject in need thereof, comprising administering to the subject a therapeutically effective amount of the oncolytic virus of claim 18.

45.-48. (canceled)

49. The method of claim 44, wherein said cancer is glioblastoma, ovarian cancer, pancreatic cancer, leukemia, lymphoma, lung cancer, breast cancer, or a brain metastatic tumor of a primary tumor.

50. (canceled)

51. The method of claim 39, further comprising administering an anticancer therapeutic.

52. A method of inducing an immune response in a subject in need thereof, said method comprising administering to said subject an effective amount of the oncolytic virus of claim 18.

53.-57. (canceled)


SEQUENCE LISTING (40 sequences)

SEQ ID NO: 1 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 2 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 3 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 4 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 5 - protein, 99 aa, Homo sapiens
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 6 - DNA, 144 bp, human alphaherpesvirus 1
ttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccga

SEQ ID NO: 7 - protein, 491 aa, Homo sapiens
EVQLVQSGAEVKKPGSSVKVSCKASGGTFSRSAIIWVRQAPGQGLEWMGGIVPMFGPPNYAQKFQGRVTITADESTNTAYMELSSLRSEDTAFYFCAGGYGIYSPEEYNGGLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPQVKFNWYVDGVQVHNAKTKPREQQYDSTYRVVSVLTVLHQNWLDGKEYKCKVSNKALPAPIEKTISKAKGQPREPQVYTLPPSREEMTKNQVSLTCLVKGFYPSDIAVEWESNDGEPENYKTTPPVLDSDGSFFLYSKLTVDKSRWQEGNVFSCSVMHEALHNHYTQKSLSLSPGDGSFFLYSKLTVDKSRWQEGNVFSSVMHEALHNHYTQKSLSLSPG

SEQ ID NO: 8 - DNA, 21 bp, Artificial Sequence
  Synthetic polynucleotide
ctgcaagagacttccatccag

SEQ ID NO: 9 - DNA, 23 bp, Artificial Sequence
  Synthetic polynucleotide
agtggtatagacaggtctgttgg

SEQ ID NO: 10 - DNA, 23 bp, Artificial Sequence
  Synthetic polynucleotide
atccacggcatactatcaacatc

SEQ ID NO: 11 - DNA, 21 bp, Artificial Sequence
  Synthetic polynucleotide
tcgtagtcatacggtgtggtg

SEQ ID NO: 12 - DNA, 22 bp, Artificial Sequence
  Synthetic polynucleotide
ccagctctgtgcaaacctaacc

SEQ ID NO: 13 - DNA, 20 bp, Artificial Sequence
  Synthetic polynucleotide
gccacgagcaagaggagaga

SEQ ID NO: 14 - DNA, 21 bp, Artificial Sequence
  Synthetic polynucleotide
tgacggcaaacatgacttcag

SEQ ID NO: 15 - DNA, 17 bp, Artificial Sequence
  Synthetic polynucleotide
ggtgccatcgggcatct

SEQ ID NO: 16 - DNA, 23 bp, Artificial Sequence
  Synthetic polynucleotide
cctgaagtgtgaagcaccaaatt

SEQ ID NO: 17 - DNA, 23 bp, Artificial Sequence
  Synthetic polynucleotide
cttcaagtccatgtttctttgca

SEQ ID NO: 18 - DNA, 22 bp, Artificial Sequence
  Synthetic polynucleotide
gaaatgccaccttttgacagtg

SEQ ID NO: 19 - DNA, 21 bp, Artificial Sequence
  Synthetic polynucleotide
tggatgctctcatcaggacag

SEQ ID NO: 20 - DNA, 22 bp, Artificial Sequence
  Synthetic polynucleotide
ttcgacacatgggataacgagg

SEQ ID NO: 21 - DNA, 21 bp, Artificial Sequence
  Synthetic polynucleotide
tttttgctgtgagtcccggag

SEQ ID NO: 22 - DNA, 21 bp, Artificial Sequence
  Synthetic polynucleotide
cctgaaccttccaaagatggc

SEQ ID NO: 23 - DNA, 21 bp, Artificial Sequence
  Synthetic polynucleotide
ttcaccaggcaagtctcctca

SEQ ID NO: 24 - DNA, 21 bp, Artificial Sequence
  Synthetic polynucleotide
atggccctgtgccttagtagt

SEQ ID NO: 25 - DNA, 22 bp, Artificial Sequence
  Synthetic polynucleotide
agctttgcattcatggtcttga

SEQ ID NO: 26 - DNA, 19 bp, Artificial Sequence
  Synthetic polynucleotide
agggacaagcctacccctc

SEQ ID NO: 27 - DNA, 20 bp, Artificial Sequence
  Synthetic polynucleotide
ctcatctcccgtcagttggt

SEQ ID NO: 28 - DNA, 20 bp, Artificial Sequence
  Synthetic polynucleotide
gtaacccgttgaaccccatt

SEQ ID NO: 29 - DNA, 20 bp, Artificial Sequence
  Synthetic polynucleotide
ccatccaatcggtagtagcg

SEQ ID NO: 30 - DNA, 66 bp, Artificial Sequence
  Synthetic polynucleotide
atggacatgagggtctctgctcagctcctggggctcctgctgctctggctctcaggagccagatgt

SEQ ID NO: 31 - DNA, 714 bp, Artificial Sequence
  Synthetic polynucleotide
gatattctgctgacccagagcccggtgattctgagcgtgagcccgggcgaacgcgtgagctttagctgccgcgcgagccagagcattggcaccaacattcattggtatcagcagcgcaccaacggcagcccgcgcctgctgattaaatatgcgagcgaaagcattagcggcattccgagccgctttagcggcagcggcagcggcaccgattttaccctgagcattaacagcgtggaaagcgaagatattgcggattattattgccagcagaacaacaactggccgaccacctttggcgcgggcaccaaactggaactgaaaggcggtggcggttctggtggcggtggctccggcggtggcggttctcaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcagagcaacgataccgcgatttattattgcgcgcgcgcgctgacctattatgattatgaatttgcgtattggggccagggcaccctggtgacc

SEQ ID NO: 32 - DNA, 696 bp, Artificial Sequence
  Synthetic polynucleotide
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgtcgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcgctctcaccgtggacaagagcaggtggcagcaggggaacgtcttctcatgctccgtgatgcatgaggctctgcacaaccactacacgcagaagagcctctccctgtctccgggtaaa

SEQ ID NO: 33 - DNA, 273 bp, Homo sapiens
atgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagc

SEQ ID NO: 34 - DNA, 45 bp, Artificial Sequence
  Synthetic polynucleotide
ggcggtggcggttctggtggcggtggctccggcggtggcggttct

SEQ ID NO: 35 - DNA, 696 bp, Artificial Sequence
  Synthetic polynucleotide
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgtaccctgcccccatcccgggatgagctgaccaagaaccaggtcagcctgtcttgcgctgtcaaaggcttctatcccagcgacatcgccgtggagtgggagagcaatgggcagccggagaacaactacaagaccacgcctcccgtgctggactccgacggctccttcaaactcgttagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - protein, 22 aa, Artificial Sequence
  Synthetic polypeptide
MDMRVSAQLLGLLLLWLSGARC

SEQ ID NO: 37 - protein, 238 aa, Artificial Sequence
  Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVT

SEQ ID NO: 38 - protein, 232 aa, Artificial Sequence
  Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 39 - protein, 15 aa, Artificial Sequence
  Synthetic polypeptide
GGGGSGGGGSGGGGS

SEQ ID NO: 40 - protein, 232 aa, Artificial Sequence
  Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK